(19) United States
(12) Patent Application Publication
(10) Pub. No.: US 2013/0196986 A1
(43) Pub. Date: Aug. 1, 2013

(54) TRIAZOLOPYRAZINONES AS P2X7 RECEPTOR ANTAGONISTS

(71) Applicants: Marc Labroli, Moorestown, NJ (US); Michael F. Czarniecki, Watchung, NJ (US); Cory Seth Poker, South Palinfield, NJ (US)

(72) Inventors: Marc Labroli, Moorestown, NJ (US); Michael F. Czarniecki, Watchung, NJ (US); Cory Seth Poker, South Palinfield, NJ (US)

(21) Appl. No.: 13/825,161

(22) PCT Filed: Sep. 16, 2011
(86) PCT No.: PCT/US11/51923

§ 371 Date: Mar. 20, 2013

Related U.S. Application Data
(60) Provisional application No. 61/384,954, filed on Sep. 21, 2010.

(51) Int. Cl. C07D 487/04; A61K 45/06; A61K 31/5377; A61K 31/4985

(52) U.S. Cl. CPC C07D 487/04; A61K 31/4985; A61K 45/06; A61K 31/5377
USPC 514/233.2; 544/350; 514/249; 544/117

(57) ABSTRACT

The invention provides certain 3,7-disubstituted-[1,2,4]triazolo[4,3-a]pyrazin-8(7H)-ones of the Formula (I), and their pharmaceutically acceptable salts, wherein R¹, R², R³, R⁸, and X are as defined herein. The invention also provides pharmaceutical compositions comprising such compounds, and methods of using the compounds for treating pain or an inflammatory disease.

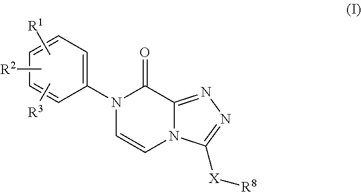

### FIELD OF THE INVENTION

The present invention relates to certain 3,7-disubstituted-[1,2,4]triazolo[4,3-a]pyrazin-8(7H)-ones of the Formula (I) (also referred to herein as the “Triazolopyrazinone Compounds” or “compounds of the Formula (I)”), compositions comprising Triazolopyrazinone Compounds, and methods of using Triazolopyrazinone Compounds for treating pain or an inflammatory disease.

### BACKGROUND OF THE INVENTION

P2X₇ receptors are activated by elevated concentrations of ATP, and function as an ion channel which is permeable to Na⁺, K⁺, and Ca⁺⁺. Brief activation of the P2X₇ receptors by an agonist results in a transient current through opening of cation channels. Prolonged or repeated exposure of P2X₇ receptors to agonist leads to a sustained current because a pore forms which is permeable to large organic or inorganic cations. Steinberg et al., J. Biol. Chem. 2626, 8884-8888 (1987). Activation of the P2X₇ receptor also results in the release of the proinflammatory cytokine interleukins IL-1β and IL-18. Guile et al., J. Med. Chem. 52, 3123-3140 (2009).

P2X₇ receptors are expressed on immune cells where they are involved in modulating immune functions, and on cells in the central nervous system. Mast cells, lymphocytes, erythrocytes, fibroblasts, peripheral macrophages, epidermal Langerhans cells, microglia, Schwann cells, and astrocytes all express P2X₇ receptors on their cell surfaces. P2X₇ receptors have been implicated as having a pathophysiological role in inflammatory responses and in chronic pain. Donnelly-Roberts et al., Br. J. Pharmacol. 151, 571-579 (2007).

Animal models support a role for modulating P2X₇ receptors to treat inflammatory diseases and pain. For example, one of the inflammatory diseases linked to the activity of P2X₇ receptors is rheumatoid arthritis, which is characterized by significant synovial inflammation and destruction of extracellular matrix and articular structures including cartilage and bone. In a mouse model of collagen-induced arthritis, animals that were deficient in the P2X₇ receptor showed both less incidence and severity of the disease as compared to wild type animals. Labasi et al., J. Immunol. 168, 6436-6445 (2002). Furthermore, mouse model studies also support a role for modulating P2X₇ receptor function in treating pain. For instance, in one such study, knockout mice lacking P2X₇ receptor function did not exhibit inflammatory or neuropathic hypersensitivity in response to mechanical and thermal stimuli. Chessell et al., Pain 114, 386-396 (2005). Thus, such studies support a role for treating or preventing inflammation and in ameliorating pain that include antagonizing P2X₇ receptor function.

Therapies that include administration of small molecule P2X₇ receptor antagonists would be particularly attractive for purposes of convenience and patient compliance. Indeed, a benzyl substituted triazole which demonstrated the ability to inhibit Ca⁺⁺ flux in activated cells, also exhibited antinociceptive activity in a rat model of neuropathic pain. Carroll et al., Bioorg. Med. Chem. Lett. 17, 4044-4048 (2007).

### SUMMARY OF THE INVENTION

In one aspect, the present invention provides compounds of Formula (I):

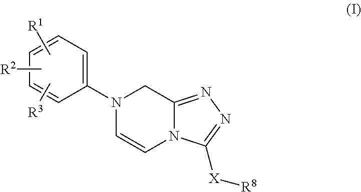

or pharmaceutically acceptable salts thereof, wherein
X is
   (i) —S(O)ₘ—, wherein m is 0, 1, or 2; or
   (ii) —N(R⁹)—;
R⁸ is —Y—R¹⁰;
   Y is
   (i) C₁-C₃ alkylene, wherein said alkylene is unsubstituted or substituted by one to two moieties independently selected from the group consisting of C₁-C₃ alkyl, and phenyl;
   (ii) —CH₂C(O)—;
   (iii) —N(H)C(O)—;
   (iv) —C(O)N(H)—;
   (v) —C(O)N(C₁-C₃ alkyl)-; or
   (vi) Y is a bond such that R⁸ is bonded directly to X;
   R¹⁰ is
   (i) C₆-C₁₀ aryl;
   (ii) fluorenyl;
   (iii) 5- to 10-membered heteroaryl, wherein said heteroaryl contains from one to three heteroatoms independently selected from the group consisting of N, O, and S;
   (iv) C₁-C₃ alkyl; or
   (v) —O—(C₁-C₆)alkyl;
   wherein said aryl, fluorenyl, or heteroaryl of R¹⁰ is unsubstituted or substituted with one or more moieties independently selected from the group consisting of halo,
      C₁-C₃ alkyl, C₁-C₃ alkoxy, C₁-C₃ fluoroalkoxy,
      C₁-C₃ fluoroalkyl, C₁-C₃ trifluoroalkylthio, cyano, carboxy,
      —C(O)—O—(C₁-C₃ alkyl), carbamyl, —C(O)—(C₁-C₃)alkyl,
      —C(O)N(H)(C₁-C₃ alkyl), —C(O)N(C₁-C₃ alkyl)₂, —NHC(O)—(C₁-C₃ alkyl), or -MR¹¹, wherein
         M is C₁-C₃ alkylene, —(C₁-C₃ alkylene)-O—, —O—, —CH₂S(O)₂—, or a bond;
         R¹¹ is
         (i) phenyl;
         (ii) 5- to 6-membered heterocyclyl having one to two heteroatoms independently selected from the group consisting of N and O; and
         (iii) 5- to 6-membered heteroaryl having one three heteroatoms independently selected from the group of N, O, and S;
         wherein said phenyl, heterocyclyl, or heteroaryl of R¹¹ is unsubstituted or substituted by one to three moieties independently selected from the group consisting of halo, C₁-C₃ alkyl, trifluoromethyl, trifluoromethoxy, C₁-C₃ alkoxy, cyano, carbamyl, amino, C₁-C₃ alkylamino, C₁-C₃ dialkylamino, acetamido, C₁-C₆ alkoxycarbonyl, and boronic acid;
R⁹ is H, C₁-C₃ alkyl, —(C₁-C₃ alkylene)-(C₃-C₇ cycloalkyl), or —(C₁-C₃ alkylene)-phenyl; and
R¹, R², and R³ are independently selected from the group consisting of hydrogen, halo, C₁-C₃ alkyl, C₁-C₃ alkoxy, trifluoromethyl, trifluoromethoxy, cyano, carbamyl, amino, C₁-C₃ alkylamino, C₁-C₃ dialkylamino, and acetamido.

### DETAILED DESCRIPTION OF THE INVENTION

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “fluoroalkyl,” “−O-alkyl,” etc.

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “therapeutically effective amount” as used herein, refers to an amount of Triazolopyrazinone Compound and/or an additional therapeutic agent, or a composition thereof that is effective in producing the desired therapeutic, ameliorative, inhibitory or preventative effect when administered to a patient suffering from pain or an inflammatory disease or disorder. In the combination therapies of the present invention, a therapeutically effective amount can refer to each individual agent or to the combination as a whole, wherein the amounts of all agents administered are together effective, but wherein the component agent of the combination may not be present individually in an effective amount.

The term “preventing,” as used herein with respect to pain or an inflammatory disease or disorder, refers to reducing the likelihood of pain or an inflammatory disease or disorder.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from 1 to 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkoxy” as used herein, refers to an —O-alkyl group, wherein an alkyl group is as defined above. Non-limiting examples of alkoxy groups include methoxy, ethoxy, n-propoxy, isopropoxy, n-butoxy and t-butoxy. An alkoxy group is bonded via its oxygen atom.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group has from 1 to 3 carbon atoms. In another embodiment, an alkylene group is branched. In another embodiment, an alkylene group is linear. In one embodiment, an alkylene group is —CH₂—. The term “C₁-C₃ alkylene” refers to an alkylene group having from 1 to 3 carbon atoms. Unless otherwise indicated, an alkylene group is unsubstituted.

The term “aryl,” as used herein, refers to an aromatic monocyclic or multicyclic ring system comprising from about 6 to about 14 carbon atoms. In one embodiment, an aryl group contains from about 6 to 10 carbon atoms (C₆-C₁₀ aryl). In another embodiment an aryl group is phenyl. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. Unless otherwise indicated, an aryl group is unsubstituted.

The term “carbamyl,” as used herein, refers to the moiety —C(O)NH₂ wherein the point of attachment to the parent moiety is through the carbonyl carbon atom.

The term “cycloalkyl,” as used herein, refers to a non-aromatic mono- or multicyclic ring system comprising from about 3 to about 10 ring carbon atoms. In one embodiment, a cycloalkyl contains from about 5 to 10 ring carbon atoms. In another embodiment, a cycloalkyl contains from about 3 to about 7 ring atoms. In another embodiment, a cycloalkyl contains from about 5 to about 6 ring atoms. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. The term “C₃-C₇ cycloalkyl” refers to a cycloalkyl group having from 3 to 7 ring carbon atoms. Unless otherwise indicated, a cycloalkyl group is unsubstituted. A ring carbon atom of a cycloalkyl group may be functionalized as a carbonyl group. An illustrative example of such a cycloalkyl group includes, but is not limited to, cyclobutanoyl.

The term “halo,” as used herein, means —F, —Cl, —Br or —I. In one embodiment, a halo group is —F or —Cl. In another embodiment, a halo group is —F.

The term “fluoroalkyl,” as used herein, refers to an alkyl group as defined above, wherein one or more of the alkyl group's hydrogen atoms has been replaced with a fluorine. In one embodiment, a fluoro group has from 1 to 6 carbon atoms. In another embodiment, a fluoroalkyl group is substituted with from 1 to 3 F atoms. Non-limiting examples of fluoroalkyl groups include —CH₂F, —CHF₂, and —CF₃. The term “C₁-C₃ fluoroalkyl” refers to a fluoroalkyl group having from 1 to 3 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered heteroaryl. In another embodiment, a heteroaryl group comprises a 5- to 6-membered heteroaryl group fused to a benzene ring. Unless otherwise indicated, a heteroaryl group is unsubstituted.

The term “heterocyclyl,” as used herein, refers to a non-aromatic saturated monocyclic or multicyclic ring system comprising 3 to about 11 ring atoms, wherein from 1 to 4 of the ring atoms are independently O, S, or N, and the remainder of the ring atoms are carbon atoms. A heterocyclyl group can be joined via a ring carbon or ring nitrogen atom. In one embodiment, a heterocyclyl group is monocyclic and has from about 3 to about 7 ring atoms. In another embodiment, a heterocyclyl group is monocyclic has from about 4 to about 7 ring atoms. In another embodiment, a heterocyclyl group is bicyclic and has from about 7 to about 11 ring atoms. In still another embodiment, a heterocyclyl group is monocyclic and has 5 or 6 ring atoms. In one embodiment, a heterocyclyl group is monocyclic. In another embodiment, a heterocyclyl group is bicyclic. The term “heterocyclyl” also encompasses a heterocyclyl group, as defined above, which is fused to an aryl (e.g., benzene) or heteroaryl ring. A heterocyclyl group can be optionally substituted by one or more “ring system substituents” which may be the same or different, and are as defined herein below. The nitrogen or sulfur atom of the heterocyclyl can be optionally oxidized to the corresponding N-oxide, S-oxide or S,S-dioxide. Non-limiting examples of monocyclic heterocyclyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, and the like, and all isomers thereof.

In one embodiment, a heterocyclyl group is a 5- to 6-membered monocyclic heterocyclyl. In another embodiment, a heterocyclyl group is a 5-membered monocyclic heterocyclyl. In another embodiment, a heterocyclyl group is a 6-membered monocyclic heterocyclyl. The term “5- to 6-membered heterocyclyl” refers to a monocyclic heterocyclyl group having from 5 to 6 ring atoms. Unless otherwise indicated, a heterocyclyl group is unsubstituted.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(N—CN)—NH₂, —C(═NH)—NH(alkyl), Y₁Y₂N—, Y₁Y₂N-alkyl-, Y₁Y₂NC(O)—, and Y₁Y₂NS(O)₂—, wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moieties are methylenedioxy, ethylenedioxy, —C(CH₃)₂— and the like.

The term “substituted” means that one or more hydrogens on the atoms of the designated are replaced with a selection from the indicated group, provided that the atoms' normal valencies under the existing circumstances are not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

When any substituent or variable occurs more than one time in any constituent or in Formula (I), its definition on each occurrence is independent of its definition at every other occurrence, unless otherwise indicated.

The term “in purified form,” as used herein, refers to the physical state of a compound after the compound is isolated from a synthetic process (e.g., from a reaction mixture), a natural source, or a combination thereof. The term “in purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention.

All stereoisomers (for example, geometric isomers, optical isomers and the like) of the present compounds (including those of the salts and solvates of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations.

The compounds of Formula (I) can form salts which are also within the scope of this invention. Reference to a compound of Formula (I) or an Triazolopyrazinone Compound herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of Formula (I) may be formed, for example, by reacting a compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quartemized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

The present invention further includes the compounds of Formula (I) in all their isolated forms. For example, the above-identified compounds are intended to encompass all forms of the compounds such as, any solvates, hydrates, stereoisomers, and tautomers thereof.

As used herein, the term “composition” is intended to encompass a product comprising the specified ingredients in the specified amounts, as well as any product which results, directly or indirectly, from combination of the specified ingredients in the specified amounts.

In the compounds of generic Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula (I). For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula (I) can be prepared without undue experimentation by conventional techniques well known to those skilled in the art or by processes analogous to those described in the Schemes and Examples herein using appropriate isotopically-enriched reagents and/or intermediates.

### Compounds of the Invention

The present invention provides Triazolopyrazinone Compounds having the Formula (I), or pharmaceutically acceptable salts thereof, wherein R¹, R², R³, R⁸, and X are as defined above for the compound of Formula (I). The compounds of Formula (IA) and (IB), as are described in detail below, are embodiments of the compound of Formula (I).

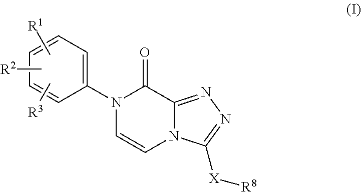

In a first embodiment of the compounds of Formula (I), X is —S(O)ₘ—, wherein m is 1 or 2.

In a second embodiment of the compound of Formula (I), X is —S(O)ₘ—, wherein m is 1.

In third embodiment of the compound of Formula (I), X is —S(O)ₘ—, wherein m is 2.

In a fourth embodiment of the compound of Formula (I), Y is

(i) C₁-C₃ alkylene, wherein said alkylene is unsubstituted or substituted by one to two moieties independently selected from the group consisting of C₁-C₃ alkyl and phenyl;

(ii)-CH₂C(O)— (wherein the carbonyl carbon atom is attached to R¹⁰); or

(iii) a bond.

In a fifth embodiment of the compound of Formula (I), Y is C₁-C₃ alkylene, wherein said alkylene is unsubstituted or substituted by one C₁-C₃ alkyl or phenyl.

In a sixth embodiment of the compound of Formula (I), Y is methylene, wherein said methylene is unsubstituted or substituted by one phenyl.

In a seventh embodiment of the compound of Formula (I), R¹⁰ is
(i) C₆-C₁₀ aryl;
(ii) fluorenyl; or
(iii) 5- to 10-membered heteroaryl, wherein said heteroaryl contains from one to three heteroatoms independently selected from the group consisting of N, O, and S;
wherein said aryl, fluorenyl, or heteroaryl of R¹⁰ is unsubstituted or substituted with one to three moieties independently selected from the group consisting of halo, C₁-C₄ alkyl, C₁-C₃ alkoxy, C₁-C₃ fluoroalkoxy, C₁-C₃ fluoroalkyl, C₁-C₃ trifluoroalkylthio, cyano, carboxy, —C(O)—O—(C₁-C₃ alkyl), carbamyl, —C(O)—(C₁-C₃)alkyl, —C(O)N(H)(C₁-C₃ alkyl), —C(O)N(C₁-C₃ alkyl)₂, —NHC(O)—(C₁-C₃ alkyl), and -MR¹¹.

In an eighth embodiment of the compound of Formula (I), if at least one M-R¹¹ is present on R¹⁰, then M is a bond or —O—; and

R¹¹ is phenyl, thienyl, or morpholinyl, wherein said phenyl or thienyl of R¹¹ is unsubstituted or independently substituted by one to two C₁-C₃ alkyl, halo, C₁-C₃ alkoxy, or CF₃.

In a ninth embodiment of the compound of Formula (I), R¹⁰ is phenyl, napthyl, fluorenyl, thienyl or isoxazolyl;

wherein said phenyl, napthyl, fluorenyl, thienyl, or isoxazolyl of R¹⁰ is unsubstituted or independently substituted with one to two moieties independently selected from the group consisting of halo, C₁-C₄ alkyl, C₁-C₃ alkoxy, C₁-C₃ fluoroalkyl, morpholinyl, phenyl, chlorophenyl, fluorophenyl, (C₁-C₄ alkyl)phenyl, C₁-C₃ alkoxy)phenyl, trifluoromethylphenyl, phenoxyphenyl, napthyl, thienyl, bromothienyl, and (C₁-C₃ alkyl)thienyl.

In a tenth embodiment of the compound of Formula (I), R¹, R², and R³ are independently selected from the group consisting of hydrogen, halo, C₁-C₃ alkyl, and trifluoromethyl.

In an eleventh embodiment of the compound of Formula (I),

X is —S(O)—, wherein m is 1 or 2;

Y is as described in the fourth embodiment;

R¹⁰ is as described in the seventh embodiment;

-MR¹¹, if present on R¹⁰, is as described in the eighth embodiment; and

R¹, R², and R³ are as described in the tenth embodiment.

In specific instances of any one of the first, second, third, fourth, fifth, sixth, seventh, eighth, ninth, or tenth embodiments:

R¹ is hydrogen,

R² and R³ are in an ortho or meta position on the illustrated phenyl ring (i.e., relative to the bond joining the phenyl ring to the pyrazinone ring), and are independently selected from the group consisting of halo, C₁-C₃ alkyl, and trifluoromethyl.

In another aspect, the compound of the formula (I) has the formula (IA)

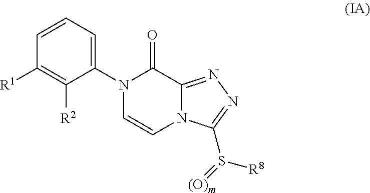

wherein
m is 1 or 2;
R⁸ is —Y—R¹⁰;
Y is methylene, wherein said methylene is unsubstituted or independently substituted by one to two moieties selected from the group consisting of C₁-C₃ alkyl and phenyl:
R¹⁰ is phenyl, napthyl, fluorenyl, thienyl or isoxazolyl;
   wherein said phenyl, napthyl, thienyl, or isoxazolyl of R¹⁰ is unsubstituted or independently substituted with one to two moieties independently selected from the group consisting of halo, C₁-C₄ alkyl, C₁-C₃ alkoxy, C₁-C₃ fluoroalkyl, morpholinyl, phenyl, chlorophenyl, fluorophenyl, (C₁-C₄ alkyl)phenyl, (C₁-C₃ alkoxy)phenyl, trifluoromethylphenyl, phenoxyphenyl, napthyl, thienyl, bromothienyl, and (C₁-C₃ alkyl)thienyl;
R¹ and R² are independently selected from the group consisting of hydrogen, halo, C₁-C₃ alkyl, and trifluoromethyl.

In a first embodiment of the compound of Formula (IA), m is 2.

In a second embodiment of the compound of Formula (IA), m is 1.

In a third embodiment of the compound of Formula (IA), R¹ is trifluoromethyl or chloro, and R² is methyl or chloro.

In a fourth embodiment of the compound of Formula (IA), Y is methylene, wherein said methylene is unsubstituted or substituted by one phenyl.

In yet another aspect, the compound of Formula (I) has the Formula (IB):

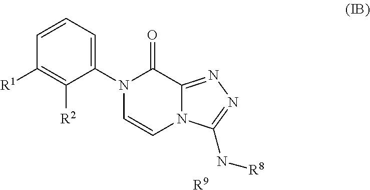

wherein

R⁸ is —Y—R¹⁰;

Y is C₁-C₄ alkylene, wherein said alkylene is unsubstituted or substituted by one to two moieties independently selected from the group consisting of C₁-C₃ alkyl and phenyl:

R¹⁰ is C₆-C₁₀ aryl;
wherein said aryl of R¹⁰ is unsubstituted or independently substituted with one to two moieties independently selected from the group consisting of halo, C₁-C₄ alkyl, C₁-C₃ alkoxy, C₁-C₃ fluoroalkyl, morpholinyl, phenyl, chlorophenyl, fluorophenyl, (C₁-C₄ alkyl)phenyl, (C₁-C₃ alkoxy)phenyl, trifluoromethylphenyl, phenoxyphenyl, napthyl, thienyl, bromothienyl, and (C₁-C₃ alkyl)thienyl;

R⁹ is selected from the group consisting of C₁-C₃ alkyl, phenyl(C₁-C₄ alkyl), and C₃-C₇ cycloalkyl;

R¹ and R² are independently selected from the group consisting of hydrogen, halo, C₁-C₃ alkyl, and trifluoromethyl.

In a first embodiment of the compound of Formula (IB), R¹⁰ is phenyl, wherein said phenyl is unsubstituted or substituted by one to two moieties independently selected from the group consisting of C₁-C₄ alkyl, C₁-C₃ alkoxy, and C₁-C₃ fluoroalkyl.

In a second embodiment of the compound of Formula (IB), R⁹ is selected from the group consisting of C₁-C₃ alkyl, benzyl, and cyclopropyl.

In a third embodiment of the compound of Formula (IB), R¹⁰ is as described in the first embodiment, and R⁹ is as described in the second embodiment.

The invention provides also provides compounds 1-171 as set forth below in Tables 3-6 in the Examples section, and pharmaceutically acceptable salts thereof. In some embodiments, the compounds are selected from the group consisting of the compounds 1-115 and 166-171 as set forth in the Tables 3, 5, and 6 below, and pharmaceutically acceptable salts thereof. In other embodiments, the compounds are selected from the group consisting of compounds 116-165 as set forth in Table 4 below, and pharmaceutically acceptable salts thereof.

The invention also provides any one of compounds 1-171 and pharmaceutically acceptable salts thereof in purified form.

### Uses of the Triazolopyrazinone Compounds

The Triazolopyrazinone Compounds are useful in human and veterinary medicine in the therapy of pain and/or an inflammatory disease.

While not being bound by any specific theory, Applicants believe that the Triazolopyrazinone Compounds are useful in treating pain or an inflammatory disease because of their P2X₇ receptor antagonist activity. The general value of the compounds of the invention in antagonizing P2X₇ receptor function can be determined, for example, using the fluorescence-based assay described below in Example 12.

### Treating Pain

In another embodiment, the present invention provides a method for treating pain in a patient, comprising administering to the patient an effective amount of a Triazolopyrazinone Compound or a pharmaceutically acceptable salt thereof. In certain embodiments, the Triazolopyrazinone Compound used in the method is selected from one of the representative compounds 1471 as set forth in Tables 3-6 in the Examples section. In specific embodiments, the Triazolopyrazinone Compound used in the method is selected from compounds 116-165 set forth in Table 4 in the Examples section.

In another embodiment, the present invention provides a Triazolopyrazinone Compound or a pharmaceutically acceptable salt thereof for use in the manufacture of a medicament for treating pain. In certain embodiments of the use, the Triazolopyrazinone Compound is selected from one of the representative compounds 1-171 as set forth in Tables 3-6 in the Examples section. In specific embodiments of the use, the Triazolopyrazinone Compound is selected from compounds 116-165 set forth in Table 4 in the Examples section.

In another embodiment, the present invention provides a Triazolopyrazinone Compound or a pharmaceutically acceptable salt thereof for use in treating pain. In certain embodiments of the use, the Triazolopyrazinone Compound is selected from one of the representative compounds 1-171 as set forth in Tables 3-6 in the Examples section. In specific embodiments of the use, the Triazolopyrazinone Compound is selected from compounds 116-165 set forth in Table 4 in the Examples section.

Non-limiting examples of pain treatable or preventable using the present methods and uses, include acute pain, back pain, chronic pain, fibromyalgia, post-herpatic neuralgia, neuropathic pain, nociceptive pain, cutaneous pain, somatic pain, visceral pain, phantom limb pain, cancer pain (including breakthrough pain), pain caused by drug therapy (such as cancer chemotherapy), headache (including migraine, tension headache, cluster headache), inflammatory pain, pain caused by diabetes, pain caused by arthritis, pain caused by injury, toothache, or pain caused by a medical procedure (such as surgery, physical therapy or radiation therapy).

In one embodiment, the pain is neuropathic pain.

In another embodiment, the pain is cancer pain.

In another embodiment, the pain is headache.

In still another embodiment, the pain is chronic pain.

In yet another embodiment, the pain is pain cause by arthritis.

In another embodiment, the pain is pain cause by diabetes.

In a further embodiment, the pain is inflammatory pain.

Neuropathic pain as used herein refers to an abnormal state of pain sensation, in which a reduction of pain threshold and the like are continued, due to functional abnormalities accompanying damage or degeneration of a nerve, plexus or perineural soft tissue, which is caused by a wound (e.g., lacerations, contusions, nerve avulsion injuries, amputation of a limb), compression (carpal tunnel syndrome, trigeminal neuralgia, tumor activity), infection, cancer, ischemic and the like, or metabolic disorders such as diabetes mellitus and the like. Neuropathic pain includes pain caused by central nerve damage, peripheral nerve damage, diabetic neuropathy, mononeuropathy or polyneuropathy. In one embodiment, the neuropathic pain is induced by diabetes.

Other examples of neuropathic pain treatable or preventable using the Triazolopyrazinone Compounds include, but are not limited to, pain caused by naturopathic therapy, pain that is resistant to naturopathic therapy, allodynia (a pain sensation induced by mechanical or thermal stimulus that does not normally provoke pain), hyperalgesia (an excessive response to a stimulus that is normally painful), hyperesthesia (an excessive response to a contact stimulus), diabetic polyneuropathy, entrapment neuropathy, central pain, labor pain, myocardial infarction pain, post-stroke pain, pancreatic pain, colic pain, muscle pain, post-operative pain, post-stroke pain, pain associated with Parkinson's disease, pain associated with intensive care, pain associated with a periodontal disease (including gingivitis and periodontitis), menstrual pain, migraine pain, persistent headaches (e.g., cluster headache or chronic tension headache), persistent pain states (e.g., fibromyalgia or myofascial pain), trigeminal neuralgia, postherpetic neuralgia, bursitis, pain associated with AIDS, pain associated with multiple sclerosis, pain due to spinal trauma and/or degeneration, burn pain, referred pain, enhanced memory of pain and neuronal mechanisms involved in coping with pain.

In specific embodiments, the Triazolopyrazinone Compounds are useful for treating or preventing allodynia or hyperalgesia.

Inflammatory pain may arise as a result of soft tissue injury including that involving the musculature (myositis) and viscera (colitis, pancreatitis, cystitis, ileitis, Crohn's disease), nerves (neuritis, radiculopathies, radioculogangionitis), arthritic conditions (e.g., rheumatoid disease and related conditions such as ankylosing spondylitis), and joint disease (including osteoarthritis).

### Treatment of an Inflammatory Disease

The Triazolopyrazinone Compounds can be useful for treating or preventing an inflammatory disease in a patient.

Accordingly, in one embodiment, the present invention provides a method for treating an inflammatory disease, comprising administering to a patient in need thereof an effective amount of a Triazolopyrazinone Compound or a pharmaceutically acceptable salt thereof. In certain embodiments, the Triazolopyrazinone Compound used in the method is selected from one of the representative compounds 1-171 as set forth in Tables 3-6 in the Examples section. In specific embodiments, the Triazolopyrazinone Compound used in the method is selected from compounds 116-165 set forth in Table 4 in the Examples section.

In another embodiment, the present invention provides a Triazolopyrazinone Compound or a pharmaceutically acceptable salt thereof for use in the manufacture of a medicament for treating an inflammatory disease. In certain embodiments of the use, the Triazolopyrazinone Compound is selected from one of the representative compounds 1-171 as set forth in Tables 3-6 in the Examples section. In specific embodiments of the use, the Triazolopyrazinone Compound is selected from compounds 116-165 set forth in Table 4 in the Examples section.

In another embodiment, the present invention provides a Triazolopyrazinone Compound or a pharmaceutically acceptable salt thereof for use in treating an inflammatory disease. In certain embodiments of the use, the Triazolopyrazinone Compound is selected from one of the representative compounds 1-171 as set forth in Tables 3-6 in the Examples section. In specific embodiments of the use, the Triazolopyrazinone Compound is selected from compounds 116-165 set forth in Table 4 in the Examples section.

Non-limiting examples of inflammatory diseases treatable or preventable using the present methods and uses, include diabetic neuropathy; arthritis, such as osteoarthritis, rheumatoid arthritis, septic arthritis, gout, pseudogout, juvenile arthritis or Still's disease; inflammatory bowel diseases, such as ileitis, Crohn's disease and ulcerative colitis; organ transplant rejection; inflammatory lung diseases such as asthma, adult respiratory distress syndrome and chronic obstructive pulmonary disease (COPD); inflammatory diseases of the eye, such as, corneal dystrophy, trachoma, uveitis and sympathetic ophthalmitis; chronic inflammatory diseases of the gum, such as gingivitis and periodontitis; inflammatory diseases of the kidney, such as glomerulonephritis and nephrosis; inflammatory diseases of the skin, such as sclerodermatitis, psoriasis and eczema; renal colic; reperfusion injury; pyrexia; ischemic injury; multiple sclerosis; systemic lupus erythematosis; periodic fever syndromes, such as chronic infantile neurological cutaneous and articular syndrome (CINCA), familial cold autoinflammatory syndrome (FCAS), Muckle-Wells Syndrome (MWS), familial Mediterranean fever (FMF) and pyrogenic arthritis, pyroderma gangrenosum and acne syndrome (PAPA); and inflammatory arthropathies, such as ankylosing spondylitis, psoriatric arthritis and Reiter's syndrome.

In addition, inflammation is a component of diabetes, metabolic disorders, cardiovascular disease, vascular complications and obesity. Thus, the present methods can alleviate the symptoms associated with such disorders as well.

The term “inflammatory disease” as used herein includes both local inflammatory responses and systemic inflammation.

In one embodiment, the inflammatory disease is arthritis.

In another embodiment, the inflammatory disease is rheumatoid arthritis.

In another embodiment, the inflammatory disease is osteoarthritis.

In still another embodiment, the inflammatory disease is psoriasis.

In still another embodiment, the inflammatory disease is asthma.

In yet another embodiment, the inflammatory disease is chronic obstructive pulmonary disease (COPD).

In another embodiment, the inflammatory disease is inflammatory bowel disease, such as ulcerative colitis and Crohn's disease.

### Combination Therapy

In one embodiment, the present invention provides a method of treating pain or an inflammatory disease, comprising administering a Triazolopyrazinone Compound or a pharmaceutically acceptable salt thereof and an additional therapeutic agent useful in treating pain or an inflammatory disease to a patient in need of such treatment.

In another embodiment, the present invention provides methods for treating a disease or disorder, such as pain or an inflammatory disease, in a patient, the method comprising administering to the patient a Triazolopyrazinone Compound, or a pharmaceutically acceptable salt thereof and at least one additional therapeutic agent that is not a Triazolopyrazinone Compound, wherein the amounts administered are together effective to treat or prevent pain or the inflammatory disease.

In one embodiment, the additional therapeutic agent is an analgesic agent.

Additional analgesic agents useful in the present methods for treating pain include, but are not limited to, non-opioid analgesics such as acetylsalicylic acid, choline magnesium trisalicylate, acetaminophen, ibuprofen, fenoprofen, diflusinal, and naproxen; opioid analgesics such as morphine, hydromorphone, methadone, levorphanol, fentanyl, oxycodone, and oxymorphone; steroids such as prednisolone, fluticasone, triamcinolone, beclomethasone, mometasone, budisamide, betamethasone, dexamethasone, prednisone, flunisolide and cortisone; COX-I inhibitors such as aspirin and piroxicam; and COX-II inhibitors such as rofecoxib, celecoxib, valdecoxib and etoricoxib.

Other analgesic agents useful in the present methods for treating pain include, but are not limited to, gabapentin, pregabalin and duloxetine.

In one embodiment, the other analgesic agent is an opioid analgesic. In another embodiment, the other analgesic agent is a non-opioid analgesic. In another embodiment, the other analgesic agent is a COX-I inhibitor. In still another embodiment, the other analgesic agent is a COX-II inhibitor. In yet another embodiment, the other analgesic agent is selected from aspirin, acetaminophen, ibuprofen, fenoprofen, naproxen, morphine, hydromorphone, methadone, levorphanol, fentanyl, oxycodone, and oxymorphone.

In one embodiment, the additional therapeutic agent is an anti-inflammatory agent.

Non-limiting examples of additional anti-inflammatory agents useful in the present methods for treating an inflammatory disease include non-steroidal anti-inflammatory agents (NSAIDs); steroidal anti-inflammatory drugs, such as cortisol, dexamethasone, prednisone, prednisolone, methylprednisone, betamethasone, beclomethasone, fludrocortisone acetate, deoxycorticosterone acetate, aldosterone, corticosterone and cortisone; agents useful for treating an inflammatory bowel disease such as IL-10, steroids, and azulfidine; and agents useful for treating rheumatoid arthritis such as methotrexate, azathioprine, cyclophosphamide, steroids and mycophenolate mofetil.

Other anti-inflammatory agents useful in the present methods for treating an inflammatory disease include, but are not limited to, rituximab, adalimumab, infliximab, etanercept, TACE inhibitors, muscarinic antagonists, kinase inhibitors, cytokine inhibitors and chemokine inhibitors.

Non-limiting examples of non-steroidal anti-inflammatory agents (NSAIDs) useful in the present methods for treating an inflammatory disease include salicylates such as aspirin, amoxipirin, benorilate, choline magnesium sulfate, diflunisal, faislamine, methyl salicylate, magnesium salicylate and salicyl salicylate; arylalkanoic acids, such as diclofenac, aceclofenac, acemetacin, bromfenac, etodolac, indomethacin, nabumetone, sulindac and tolmetin; profens, such as ibuprofen, carprofen, fenbufen, fenoprofen, flurbiprofen, ketoprofen, ketorolac, loxoprofen, naproxen, tiaprofenic acid and suprofen; fenamic acids, such as mefenamic acid and meclofenamic acid; pyrazolidine derivatives, such as phenylbutazone, azapropazone, metamizole, oxyphenbutazone and sulfinprazone; oxicams, such as piroxicam, lornoxicam, meloxicam and tenoxicam; COX-2 inhibitors, such as celecoxib, rofecoxib, etoricoxib, lumiracoxib, parecoxib and valdecoxib; sulfonalides, such as nimesulfide; licofelone; omega-3 fatty acids; and PDE inhibitors.

In one embodiment, the NSAID is a profen or a salicylate.

In another embodiment, the NSAID is a COX-2 inhibitor.

In one embodiment, the additional therapeutic agent is an anti-asthmatic agent.

Non-limiting examples of anti-asthmatic agents useful in the present methods for treating asthma include beta-2 adrenoceptor agonists, such as salmeterol, formoterol, bambuterol, albuterol, salbutamol, levalbuterol, terbutaline and bitolterol; ephedrine; ipatropium bromide; glucocorticoids, such as ciclesonide, beclomethasone, budesonide, funisolide, futicasone, mometasone and triamcinolone; leukotriene modifiers, such as montelukast, zafirlukast, pranlukast and zileuton; mast cell stabilizers, such as cromolyn and nedocromil; anticholinergics, such as ipatropium, glycopyrrolate, atropine, oxitropium and tiotropium; methylxanthines, such as theophylline and aminophylline; an antihistamine; an IgE, such as omalizumab; methotrexate; tianeptine; steroids such as prednisone, prednisolone, methylprednisone, dexamethasone and hydrocortisone; beta-agonists, such as epinephrine, isoetharine, isoproterenol and metaproterenol; inhalation anesthetics, such as isoflurane, halothane and enflurane; magnesium sulfate; heliox, which is a mixture of helium and oxygen; and expectorants, such as guaifenesin.

In one embodiment, the inflammatory disease treated using the combination therapies of the present invention is asthma. In another embodiment, the inflammatory disease is arthritis. In still another embodiment, the inflammatory disease is rheumatoid arthritis or osteoarthritis. In yet another embodiment, the inflammatory disease is COPD. In a further embodiment, the inflammatory disease is an inflammatory bowel disease.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the Triazolopyrazinone Compound is administered during a time when the additional therapeutic agent(s) exert their prophylactic or therapeutic effect, or vice versa.

In another embodiment, the Triazolopyrazinone Compound and the additional therapeutic agent(s) are administered in doses commonly employed when such agents are used as monotherapy for treating the disorder.

In another embodiment, the Triazolopyrazinone Compound and the additional therapeutic agent(s) are administered in doses lower than the doses commonly employed when such agents are used as monotherapy for treating the disorder.

In still another embodiment, the o the Triazolopyrazinone Compound and the additional therapeutic agent(s) act synergistically and are administered in doses lower than the doses commonly employed when such agents are used as monotherapy for treating the disorder.

In one embodiment, the Triazolopyrazinone Compound and the additional therapeutic agent(s) are present in the same composition. In one embodiment, this composition is suitable for oral administration. In another embodiment, this composition is suitable for intravenous administration.

The Triazolopyrazinone Compound and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of the Triazolopyrazinone Compound and the additional therapeutic agent(s) may inhibit the resistance of a disorder to these agents.

In one embodiment, the additional therapeutic agent is used at its known therapeutically effective dose. In another embodiment, the additional therapeutic agent is used at its normally prescribed dosage. In another embodiment, the additional therapeutic agent is used at less than its normally prescribed dosage or its known therapeutically effective dose.

The doses and dosage regimen of the other agents used in the combination therapies of the present invention for the treatment or prevention of a disease or disorder can be determined by the attending clinician, taking into consideration the approved doses and dosage regimen in the package insert; the age, sex and general health of the patient; and the type and severity of the viral infection or related disease or disorder. When administered in combination, the Triazolopyrazinone Compound and the other agent(s) for treating diseases or disorders listed above can be administered simultaneously or sequentially. This particularly useful when the components of the combination are given on different dosing schedules, e.g., one component is administered once daily and another component every six hours, or when the preferred pharmaceutical compositions are different, e.g., one is a tablet and one is a capsule. A kit comprising the separate dosage forms is therefore advantageous.

Generally, a total daily dosage of the one or more Triazolopyrazinone Compound and the additional therapeutic agent(s) can when administered as combination therapy, range from about 0.1 to about 2000 mg per day, although variations will necessarily occur depending on the target of the therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

### Compositions and Administration

This invention is also directed to pharmaceutical compositions which comprise at least one Triazolopyrazinone Compound, or a pharmaceutically acceptable salt of said compound and at least one pharmaceutically acceptable carrier.

When administered to a patient, the Triazolopyrazinone Compounds can be administered as a component of a composition that comprises a pharmaceutically acceptable carrier or vehicle. The present invention provides pharmaceutical compositions comprising an effective amount of at least one Triazolopyrazinone Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. Examples of pharmaceutically acceptable carriers and methods of manufacture for various compositions may be found in A. Germ=(ed.), Remington's Pharmaceutical Sciences, 18^{th} Edition, (1990), Mack Publishing Co., Easton, Pa. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

The Triazolopyrazinone Compounds of the present invention may also be delivered transdermally. The transdermal compositions can take the form of creams, lotions, aerosols and/or emulsions and can be included in a transdermal patch of the matrix or reservoir type as are conventional in the art for this purpose.

Additionally, the compositions of the present invention may be formulated in sustained release form to provide the rate controlled release of any one or more of the components or active ingredients to optimize therapeutic effects, e.g., ameliorating pain or inflammation. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the Triazolopyrazinone Compound is administered orally.

In another embodiment, the Triazolopyrazinone Compound is administered intravenously.

In another embodiment, the Triazolopyrazinone Compound is administered topically.

In still another embodiment, the Triazolopyrazinone Compounds is administered sublingually.

In one embodiment, a pharmaceutical preparation comprising at least one Triazolopyrazinone Compound is in unit dosage form. In such form, the preparation is subdivided into unit doses containing therapeutically effective amounts of the active components.

Compositions can be prepared according to conventional mixing, granulating or coating methods, respectively, and the present compositions can contain, in one embodiment, from about 0.1% to about 99% of the Triazolopyrazinone Compound(s) by weight or volume. In various embodiments, the present compositions can contain, in one embodiment, from about 1% to about 70% or from about 5% to about 60% of the Triazolopyrazinone Compound(s) by weight or volume.

The quantity of active compound in a unit dose of preparation is from about 0.1 to about 2000 mg of the Triazolopyrazinone Compound. Variations will necessarily occur depending on the patient and the route of administration. In one embodiment, the unit dose dosage is from about 0.2 to about 1000 mg. In another embodiment, the unit dose dosage is from about 1 to about 500 mg. In another embodiment, the unit dose dosage is from about 1 to about 100 mg/day. In still another embodiment, the unit dose dosage is from about 1 to about 50 mg. In yet another embodiment, the unit dose dosage is from about 1 to about 10 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the Triazolopyrazinone Compound will be regulated according to the judgment of the attending clinician considering such factors as age, condition and size of the patient as well as severity of the symptoms being treated. A typical recommended daily dosage regimen for oral administration can range from about 1 mg/day to about 300 mg/day, preferably 1 mg/day to 75 mg/day, in two to four divided doses.

The compositions of the invention can further comprise one or more additional therapeutic agents, selected from those specified above. Accordingly, in one embodiment, the present invention provides compositions comprising: (i) at least one Triazolopyrazinone Compound or a pharmaceutically acceptable salt thereof; (ii) one or more additional therapeutic agents, e.g., anti-inflammatory agents, that are not Triazolopyrazinone Compounds; and (iii) a pharmaceutically acceptable carrier, wherein the amounts in the composition are together effective to treat pain or an inflammatory disease.

### Kits

Another aspect of this invention is a kit comprising a therapeutically effective amount of the Triazolopyrazinone Compound, or a pharmaceutically acceptable salt of said compound, and a pharmaceutically acceptable carrier, vehicle or diluent.

Yet another aspect of this invention is a kit comprising an amount of the Triazolopyrazinone Compound, or a pharmaceutically acceptable salt of said compound and an amount of at least one additional therapeutic agent listed above, wherein the amounts of the two or more active ingredients result in a desired therapeutic effect. In one embodiment, the Triazolopyrazinone Compound and the at least one additional therapeutic agent are provided in the same container. In one embodiment, the Triazolopyrazinone Compound and the at least one additional therapeutic agent are provided in separate containers.

### EXAMPLES

Solvents, reagents, and intermediates that are commercially available were used as received. Reagents and intermediates that are not commercially available were prepared in the manner as described below. Where LC/MS data are presented, analyses was performed using a Phenomenex Gemini C-18 column (50×4.6 mm, 5 micron) running a gradient of 0.05% Trifluoroacetic acid in water (A) and acetonitrile (B) (90:10 to 5:95 A:B over 5 min, 1.0 mL/min) with analysis by PE SCIEX API-150EX single quadrupole mass spectrometer, or an Agilent Zorbax SB-C18 column (3.0×50 mm, 1.8 micron) running a gradient of 0.1% Trifluoroacetic acid in water (A) or acetonitrile (B) (90:10 A:B for 0.3 min, 90:10 to 5:95 A:B over 5.1 min, 5:95 A:B for 1.2 min, 1.0 mL/min) with analysis by an Agilent 6140 quadrupole mass spectrometer. The observed parent ion is given. Where the description indicates the reaction mixture was purified by HPLC, the description refers to using a preparative Agilent 1100 Series LC/MSD SL system: Column Reverse Phase—Varian Pursuit XRs 10 μC-18 250×21.2 mm; elution with gradient Acetonitrile/water with 0.1% TFA or 0.2% formic acid. The desired product was detected and collected by a mass-triggered automatic sample collector. Flash column chromatography was performed using pre-packed normal phase silica from Varian.

The following solvents, reagents and reaction conditions may be referred to by their abbreviations:

° C.: degrees Celsius

g: grams

h: hours

LC-MS: Liquid chromatography-mass spectrometry

mL: milliliter

mg: milligram

mmol: millimolar

M: Molar

MW: molecular weight

### Example 1

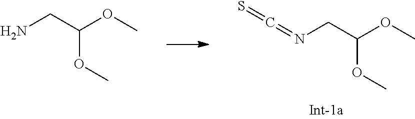

Into a 1-neck round-bottom flask was added aminoacetaldehyde dimethyl acetal (15 mL, 140 mmol) and triethylamine (38.4 mL, 275 mmol) in methylene chloride (354 mL). The solution was cooled to −10° C. and treated dropwise with a solution of carbonothioic dichloride (12.60 mL, 165.2 mmol) in methylene chloride (55 mL). The solution was stirred at −10° C. for 2 hours and then gradually warmed to room temperature and stirred overnight. The solution was concentrated and the residue was partitioned between ether (100 mL) and water (100 mL). The aqueous layer was further extracted with ether (2×50 mL). The combined organic extracts were washed with brine (50 mL), dried (Na₂SO₄), filtered and concentrated under reduced pressure to yield Int-1a (17.5 g, 86% yield) as a dark red liquid. The reaction was used directly in the next step without further purification.

### Example 2

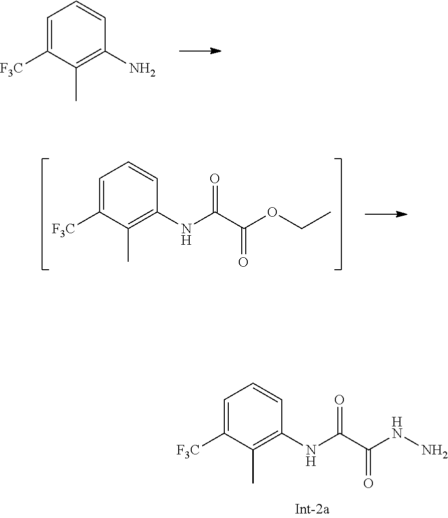

Into a 1-neck round-bottom flask was added 3-amino-2-methylbenzotrifluoride (2.50 g, 14.3 mmol), ethyl oxalyl chloride (1.75 mL, 15.7 mmol) and triethylamine (2.19 mL, 15.7 mmol) in tetrahydrofuran (28.5 mL). The solution was stirred at room temperature for 2 hours. The mixture was filtered and the precipitate was washed with tetrahydrofuran (2×4 mL). The combined organic layers were concentrated to yield a white solid. The solid was dissolved in ethanol (118 mL) at room temperature and treated with hydrazine hydrate (2.08 mL, 42.8 mmol) dropwise. The mixture was stirred at room temperature overnight. The mixture was concentrated and dried under vacuum to yield Int-2a (3.72 g, 100% yield) as a white solid. The solid was used directly in the next step without further purification.

By essentially the same procedure set forth in above Example 2, the compounds given in Column 2 of Table 1 were prepared.

TABLE 1

| Intermediate | Column 2 |
|---|---|
| Int-2a | 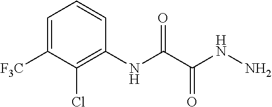 [SMILES: NNC(=O)C(=O)Nc1cccc(C(F)(F)F)c1Cl] |
| Int-2b | 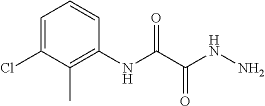 |
| Int-2c | 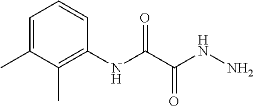 |
| Int-2d | 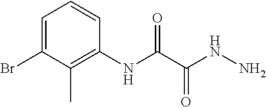 |
| Int-2e | 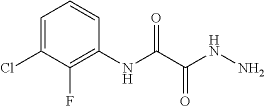 [SMILES: NNC(=O)C(=O)Nc1cccc(Cl)c1F] |
| Int-2f | 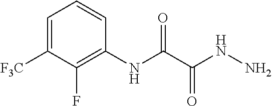 [SMILES: NNC(=O)C(=O)Nc1cccc(C(F)(F)F)c1F] |
| Int-2g | 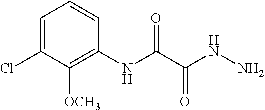 [SMILES: COc1c(Cl)cccc1NC(=O)C(=O)NN] |
| Int-2f | 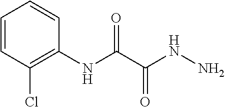 [SMILES: NNC(=O)C(=O)Nc1ccccc1Cl] |
| Int-2h | 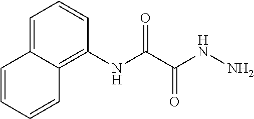 [SMILES: NNC(=O)C(=O)Nc1cccc2ccccc12] |
| Int-2i | 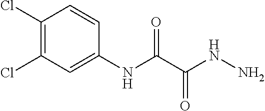 [SMILES: NNC(=O)C(=O)Nc1ccc(Cl)c(Cl)c1] |
| Int-2j | 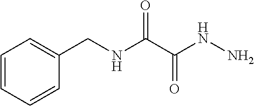 [SMILES: NNC(=O)C(=O)NCc1ccccc1] |

### Example 3

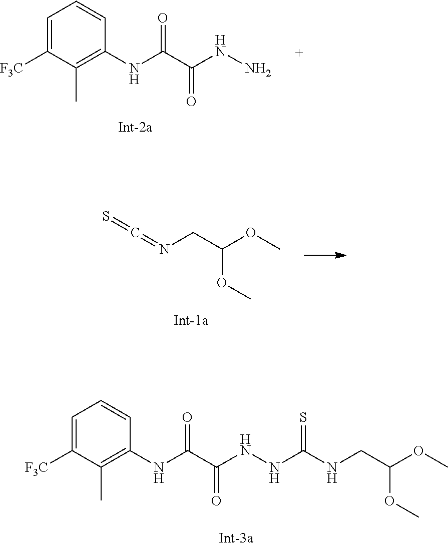

To a solution of Int-2a (7.85 g, 25.55 mmol) suspended in ethanol (150 mL) was added a solution of Int-1a (3.91 g, 26.32 mmol) in tetrahydrofuran (50 mL) dropwise, resulting in a light yellow-brown suspension. The reaction was immersed in an oil bath preheated to 55° C. and stirred overnight. The reaction was concentrated to yield Int-3a (11.24 g, 80%) as a light brown solid which was used directly in the next step without further purification.

### Example 4

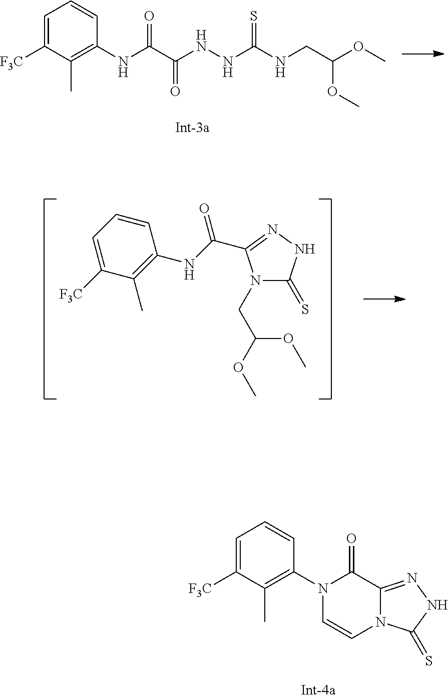

Into a 1-neck round-bottom flask was added Int-3a (1.24 g, 3.05 mmol) in water (12 mL). To the solution was added sodium bicarbonate (0.28 g, 3.35 mmol). The solution was heated at 100° C. for 1 hour. The solution was cooled to room temperature and concentrated under vacuum. The residue was dissolved in 4 M of hydrogen chloride in dioxane (20 mL) and was heated at 100° C. for 2 days. The mixture was concentrated. The residue was diluted with water (20 mL) and stirred at room temperature for 2 hours. The mixture was filtered and the precipitate was washed with water (2×3 mL) and dried under vacuum at 50° C. overnight. The residue was diluted with water (30 mL) and stirred at room temperature for 2 hours. The precipitate was filtered and dried to yield Int-4-a (0.96 g, 97% yield) as a light brown solid which was used directly in the next step without further purification.

By essentially the same procedure set forth in Example 4, the compounds given in Column 2 of Table 2 were prepared.

TABLE 2

| Preparative Example | Column 2 |
|---|---|
| Int-4a | 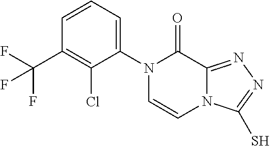 [SMILES: O=c1c2nnc(S)n2ccn1-c1cccc(C(F)(F)F)c1Cl] |
| Int-4b | 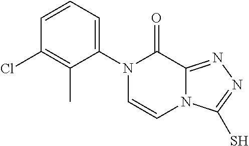 [SMILES: Cc1c(Cl)cccc1-n1ccn2c(S)nnc2c1=O] |
| Int-4c | 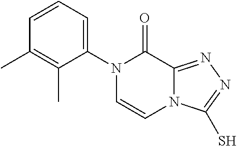 [SMILES: Cc1cccc(-n2ccn3c(S)nnc3c2=O)c1C] |
| Int-4d | 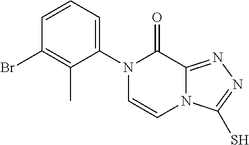 [SMILES: Cc1c(Br)cccc1-n1ccn2c(S)nnc2c1=O] |
| Int-4e | 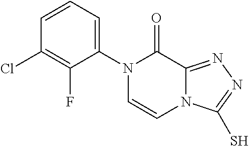 [SMILES: O=c1c2nnc(S)n2ccn1-c1cccc(Cl)c1F] |
| Int-4f | 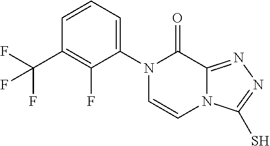 [SMILES: O=c1c2nnc(S)n2ccn1-c1cccc(C(F)(F)F)c1F] |
| Int-4g | 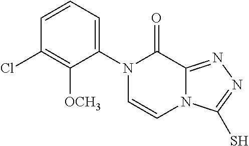 [SMILES: COc1c(Cl)cccc1-n1ccn2c(S)nnc2c1=O] |
| Int-4h | 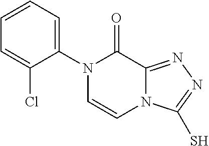 [SMILES: O=c1c2nnc(S)n2ccn1-c1ccccc1Cl] |
| Int-4i | 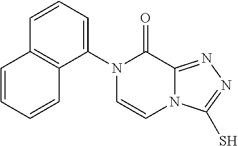 [SMILES: O=c1c2nnc(S)n2ccn1-c1cccc2ccccc12] |
| Int-4j | 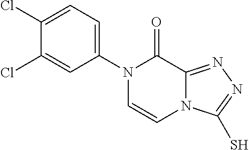 [SMILES: O=c1c2nnc(S)n2ccn1-c1ccc(Cl)c(Cl)c1] |
| Int-4k | 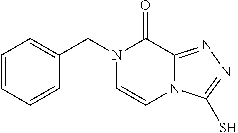 [SMILES: O=c1c2nnc(S)n2ccn1Cc1ccccc1] |

### Example 5

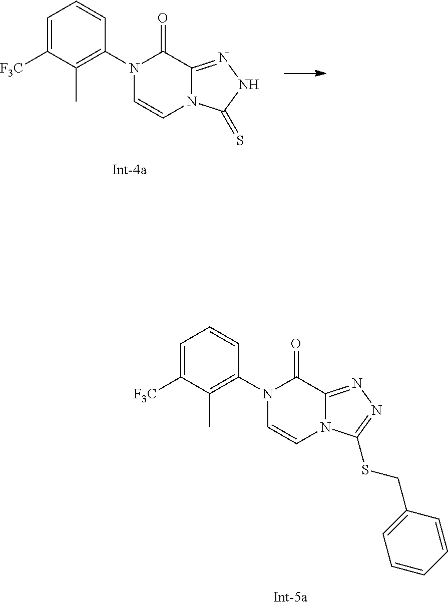 [SMILES: Cc1c(-n2ccn3c(=S)[nH]nc3c2=O)cccc1C(F)(F)F ; Cc1c(-n2ccn3c(SCc4ccccc4)nnc3c2=O)cccc1C(F)(F)F]

Into a 1-neck round-bottom flask was added Int-4-a (0.20 g, 0.61 mmol), benzyl bromide (0.11 mL, 0.92 mmol) and triethylamine (0.17 mL, 1.22 mmol) in N,N-dimethylformamide (0.92 mL). The solution was heated at 90° C. for 1.5 hours. The solution was cooled to room temperature. The brown, homogeneous solution was triturated with water (1.8 mL) and the resulting precipitate was filtered and dried under vacuum. The compound was purified on a 2000 micron preparative plate using 10% acetone in methylene chloride as eluent to yield compound 1 (0.089 g, 35%) as a white solid. LC-MS: MW [MH⁺]=417.

By essentially the same procedures set forth in Examples 1-5, the compounds given in Column 2 of Table 3, i.e., compounds 1-115 were prepared.

TABLE 3

| | | | 1. % Yield | |
|---|---|---|---|---|
| | | | 2. LC-MS: | |
| Ex- | | | MW | P2X₇ Ca Flux |
| ample | Structural Formula | Chemical Name | [MH⁺] | hu IC₅₀ nM |
| 1 | 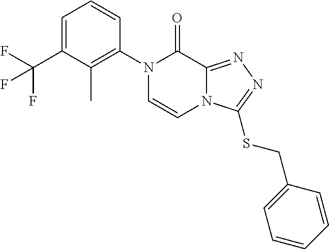 [SMILES: Cc1c(-n2ccn3c(SCc4ccccc4)nnc3c2=O)cccc1C(F)(F)F] | 3-(benzylthio)-7-(2-methyl-3- (trifluoromethyl)phenyl)- [1,2,4]triazolo[4,3-a]pyrazin-8(7H)-one | 1.35% 2.417 | 38.2 |
| 2 | 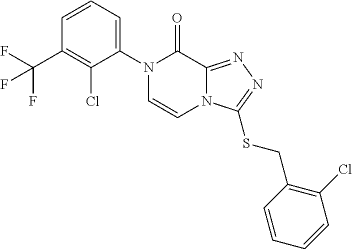 [SMILES: O=c1c2nnc(SCc3ccccc3Cl)n2ccn1-c1cccc(C(F)(F)F)c1Cl] | 7-(3-chloro-2-methylphenyl)-3-(3,4- difluorobenzylthio)-[1,2,4]triazolo[4,3- a]pyrazin-8(7H)-one | 1.43% 2.472 | 893.4 |
| 3 | 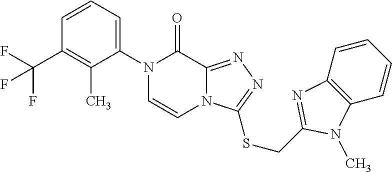 [SMILES: Cc1c(-n2ccn3c(SCc4nc5ccccc5n4C)nnc3c2=O)cccc1C(F)(F)F] | 3-((1-methyl-1H-benzo[d]imidazol-2- yl)methylthio)-7-(2-methyl-3- (trifluoromethyl)phenyl)- [1,2,4]triazolo[4,3-a]pyrazin-8(7H)-one | 1.50% 2.471 | 5667 |
| 4 | 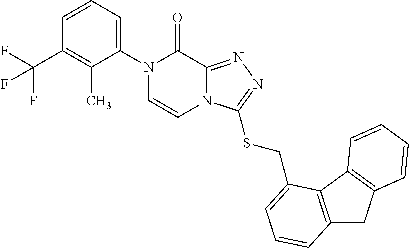 [SMILES: Cc1c(-n2ccn3c(SCc4cccc5c4-c4ccccc4C5)nnc3c2=O)cccc1C(F)(F)F] | 3-((9H-fluoren-4-yl)methylthio)-7-(2- methyl-3-(trifluoromethyl)phenyl)- [1,2,4]triazolo[4,3-a]pyrazin-8(7H)-one | 1.62% 2.505 | 263.5 |
| 5 | 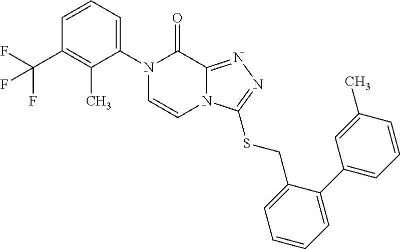 [SMILES: Cc1cccc(-c2ccccc2CSc2nnc3c(=O)n(-c4cccc(C(F)(F)F)c4C)ccn23)c1] | 7-(2-methyl-3-(trifluorornethyl)phenyl)- 3-((3'-methylbiphenyl-2-yl)methylthio)- [1,2,4]triazolo[4,3-a]pyrazin-8(7H)-one | 1.58% 2.507 | 30.4 |
| 6 | 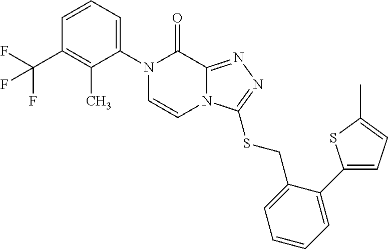 [SMILES: Cc1ccc(-c2ccccc2CSc2nnc3c(=O)n(-c4cccc(C(F)(F)F)c4C)ccn23)s1] | 7-(2-methyl-3-(trifluoromethyl)phenyl)- 3-(2-(5-methylthiophen-2- yl)benzylthio)-[1,2,4]triazolo[4,3- a]pyrazin-8(7H)-one | 1.64% 2.513 | 37.0 |
| 7 | 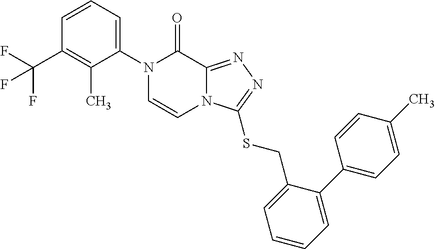 [SMILES: Cc1ccc(-c2ccccc2CSc2nnc3c(=O)n(-c4cccc(C(F)(F)F)c4C)ccn23)cc1] | 7-(2-methyl-3-(trifluoromethyl)phenyl)- 3-((4′-methylbiphenyl-2-yl)methylthio)- [1,2,4]triazolo[4,3-a]pyrazin-8(7H)-one | 1.61% 2.507 | 66.7 |
| 8 | 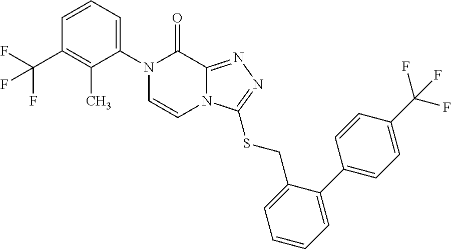 [SMILES: Cc1c(-n2ccn3c(SCc4ccccc4-c4ccc(C(F)(F)F)cc4)nnc3c2=O)cccc1C(F)(F)F] | 7-(2-methyl-3-(trifluoromethyl)phenyl)- 3-((4′-(trifluoromethyl)biphenyl-2- yl)rnethylthio)-[1,2,4]triazolo[4,3- a]pyrazin-8(7H)-one | 1.62% 2.561 | 905.4 |
| 9 | 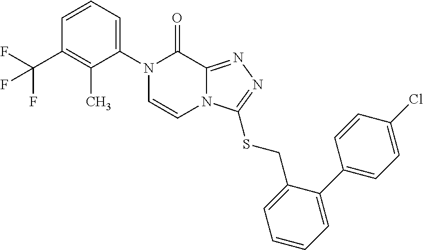 [SMILES: Cc1c(-n2ccn3c(SCc4ccccc4-c4ccc(Cl)cc4)nnc3c2=O)cccc1C(F)(F)F] | 3-((4′-chlorobiphenyl-2-yl)nrethylthio)- 7-(2-methyl-3-(trifluoromethyl)phenyl)- [1,2,4]triazolo[4,3-a]pyrazin-8(7H)-one | 1.59% 2.527 | 102 |
| 10 | 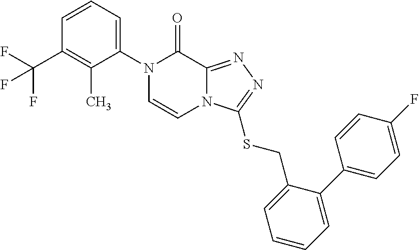 [SMILES: Cc1c(-n2ccn3c(SCc4ccccc4-c4ccc(F)cc4)nnc3c2=O)cccc1C(F)(F)F] | 3-((4′-fluorobiphenyl-2-yl)rnethylthio)- 7-(2-methyl-3-(trifluoromethyl)phenyl)- [1,2,4]triazolo[4,3-a]pyrazin-8(7H)-one | 1.64% 2.511 | 42.6 |
| 11 | 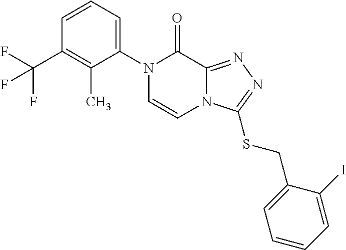 [SMILES: Cc1c(-n2ccn3c(SCc4ccccc4I)nnc3c2=O)cccc1C(F)(F)F] | 3-(2-iodobenzylthio)-7-(2-methyl-3- (trifluoromethyl)phenyl- [1,2,4]triazolo[4,3-a]pyrazin-8(7H)-one | 1.55% 2.543 | 105.8 |
| 12 | 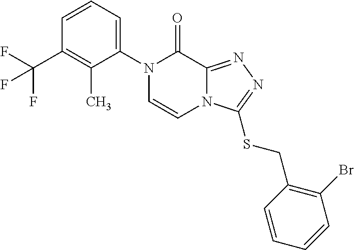 [SMILES: Cc1c(-n2ccn3c(SCc4ccccc4Br)nnc3c2=O)cccc1C(F)(F)F] | 3-(2-bromobenzylthio)-7-(2-methyl-3- (trifluoromethyl)phenyl)- [1,2,4]triazolo[4,3-a]pyrazin-8(7H)-one | 1.64% 2.497 | 107.4 |
| 13 | 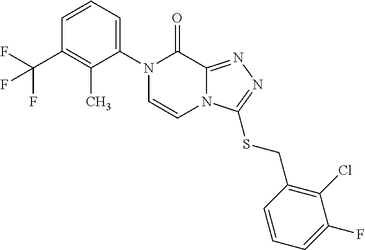 [SMILES: Cc1c(-n2ccn3c(SCc4cccc(F)c4Cl)nnc3c2=O)cccc1C(F)(F)F] | 3-(2-chloro-3-fluorobenzylthio)-7-(2- methyl-3-(trifluoromethyl)phenyl)- [1,2,4]triazolo[4,3-a]pyrazin-8(7H)-one | 1.64% 2.469 | 129.8 |
| 14 | 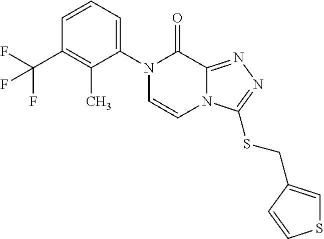 [SMILES: Cc1c(-n2ccn3c(SCc4ccsc4)nnc3c2=O)cccc1C(F)(F)F] | 7-(2-methyl-3-(trifluoromethyl)phenyl)- 3-(thiophen-3-ylmethylthio)- [1,2,4]triazolo[4,3-a]pyrazin-8(7H)-one | 1.18% 2.423 | 38.1 |
| 15 | 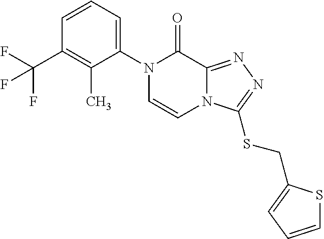 [SMILES: Cc1c(-n2ccn3c(SCc4cccs4)nnc3c2=O)cccc1C(F)(F)F] | 7-(2-methyl-3-(trifluoromethyl)phenyl)- 3-(thiophen-2-ylmethylthio)- [1,2,4]triazolo[4,3-a]pyrazin-8(7H)-one | 1.58% 2.423 | 30.4 |
| 16 | 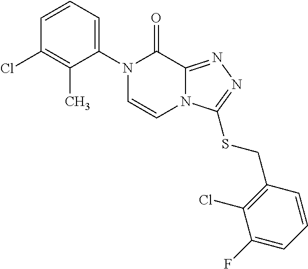 [SMILES: Cc1c(Cl)cccc1-n1ccn2c(SCc3cccc(F)c3Cl)nnc2c1=O] | 7-(3-chloro-2-methylphenyl)-3-(2- chloro-3-fluorobenzylthio)- [1,2,4]triazolo[4,3-a]pyrazin-8(7H)-one | 1.84% 2.436 | 285.7 |
| 17 | 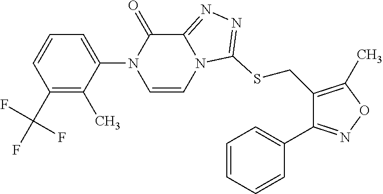 [SMILES: Cc1onc(-c2ccccc2)c1CSc1nnc2c(=O)n(-c3cccc(C(F)(F)F)c3C)ccn12] | 7-(2-methyl-3-(trifluoromethyl)phenyl)- 3-((5-methyl-3-phenylisoxazo1-4- yl)methylthio)-[1,2,4]triazolo[4,3- a]pyrazin-8(7H)-one | 1.36% 2.498 | 543.3 |
| 18 | 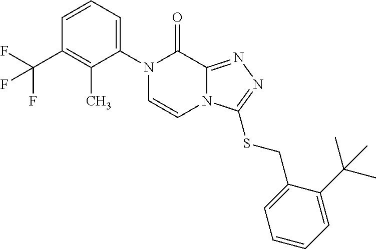 [SMILES: Cc1c(-n2ccn3c(SCc4ccccc4C(C)(C)C)nnc3c2=O)cccc1C(F)(F)F] | 3-(2-tert-butylbenzylthio)-7-(2-methyl- 3-(trifluoromethyl)phenyl)- [1,2,4]triazolo[4,3-a]pyrazin-8(7H)-one | 1.63% 2.473 | 56.9 |
| 19 | 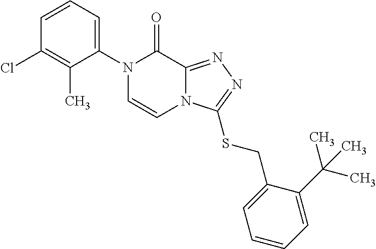 [SMILES: Cc1c(Cl)cccc1-n1ccn2c(SCc3ccccc3C(C)(C)C)nnc2c1=O] | 3-(2-tent-butylbenzylthio)-7-(3-chloro-2- methylphenyl)-[1,2,4]triazolo[4,3- a]pyrazin-8(7H)-one | 1.92% 2.439 | 165.0 |
| 20 | 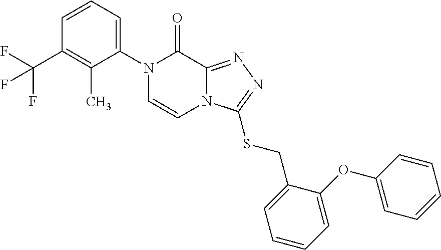 [SMILES: Cc1c(-n2ccn3c(SCc4ccccc4Oc4ccccc4)nnc3c2=O)cccc1C(F)(F)F] | 7-(2-methyl-3-(trifluoromethyl)phenyl)- 3-(2-phenoxybenzylthio)- [1,2,4]triazolo[4,3-a]pyrazin-8(7H)-one | 1.51% 2.509 | 143.5 |
| 21 | 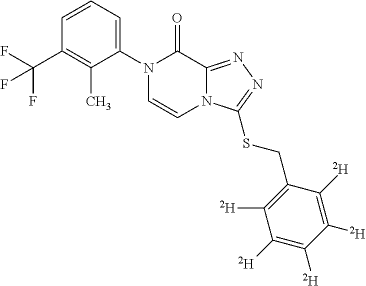 [SMILES: [2H]c1c([2H])c([2H])c(CSc2nnc3c(=O)n(-c4cccc(C(F)(F)F)c4C)ccn23)c([2H])c1[2H]] | deuterated-7-[2-methyl-3- (trifluoromethyl)phenyl]-3-[(phenyl- (d5)-methy)thio]-1,2,4-triazolo[4,3- a]pyrazin-8(7H)-one | 1.58% 2.422 | 84.9 |
| 22 | 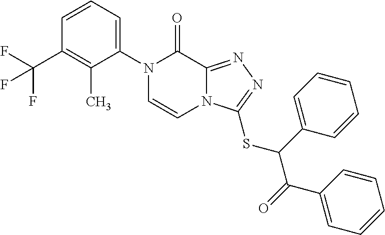 [SMILES: Cc1c(-n2ccn3c(SC(C(=O)c4ccccc4)c4ccccc4)nnc3c2=O)cccc1C(F)(F)F] | 7-(2-methyl-3-(trifluoromethyl)phenyl)- 3-(2-oxo-1,2-diphenylethylthio)- [1,2,4]triazolo[4,3-a]pyrazin-8(7H)-one | 1.47% 2.521 | 1449 |
| 23 | 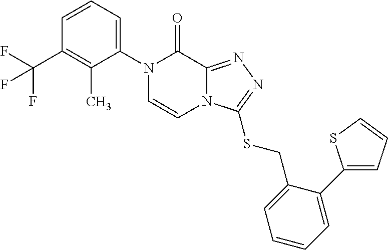 [SMILES: Cc1c(-n2ccn3c(SCc4ccccc4-c4cccs4)nnc3c2=O)cccc1C(F)(F)F] | 7-(2-methyl-3-(trifluoromethyl)phenyl)- 3-(2-(thiophen-2-yl)benzylthio)- [1,2,4]triazolo[4,3-a]pyrazin-8(7H)-one | 1.70% 2.499 | 40.4 |
| 24 | 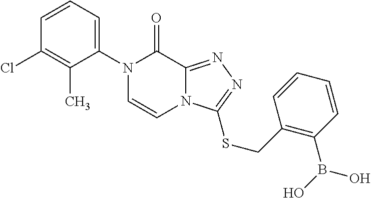 [SMILES: Cc1c(Cl)cccc1-n1ccn2c(SCc3ccccc3B(O)O)nnc2c1=O] | 2-((7-(3-chloro-2-methylphenyl)-8-oxo- 7,8-dihydro-[1,2,4]triazolo[4,3- a]pyrazin-3-ylthio)rnethyl)phenylboronic acid | 1.38% 2.427 | 1011 |
| 25 | 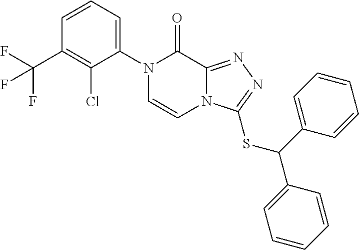 [SMILES: O=c1c2nnc(SC(c3ccccc3)c3ccccc3)n2ccn1-c1cccc(C(F)(F)F)c1Cl] | 3-(benzhydrylthio)-7-(2-chloro-3- (trifluoromethyl)phenyl)- [1,2,4]triazolo[4,3-a]pyrazin-8(7H)-one | 1.51% 2.513 | 41.7 |
| 26 | 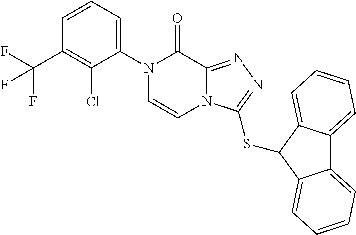 [SMILES: O=c1c2nnc(SC3c4ccccc4-c4ccccc43)n2ccn1-c1cccc(C(F)(F)F)c1Cl] | 3-(9H-fluoren-9-ylthio)-7-(2-chloro-3- (trifluoromethyl)phenyl)- [1,2,4]triazolo[4,3-a]pyrazin-8(7H)-one | 1.31% 2.511 | 47.0 |
| 27 | 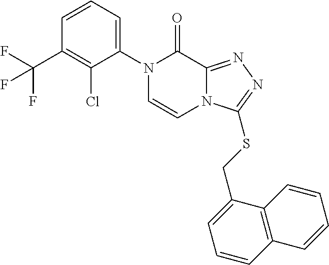 [SMILES: O=c1c2nnc(SCc3cccc4ccccc34)n2ccn1-c1cccc(C(F)(F)F)c1Cl] | 7-(2-chloro-3-(trifluoromethyl)phenyl)- 3-(naphthalen-1-ylmethylthio)- [1,2,4]triazolo[4,3-a]pyrazin-8(7H)-one | 1.42% 2.487 | 166.1 |
| 28 | 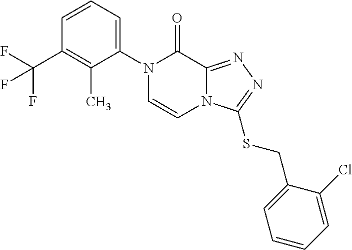 [SMILES: Cc1c(-n2ccn3c(SCc4ccccc4Cl)nnc3c2=O)cccc1C(F)(F)F] | 3-(2-chlorobenzylthio)-7-(2-methyl-3- (trifluoromethyl)phenyl)- [1,2,4]triazolo[4,3-a]pyrazin-8(7H)-one | 1.57% 2.451 | 148.2 |
| 29 | 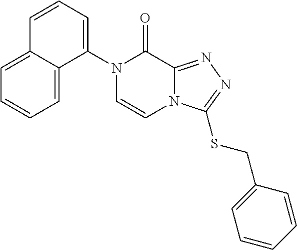 [SMILES: O=c1c2nnc(SCc3ccccc3)n2ccn1-c1cccc2ccccc12] | 3-(benzylthio)-7-(naphthalen-1-yl)- [1,2,4]-triazolo[4,3-a]pyrazin-8(7H)-one | 1.49% 2.385 | 3629 |
| 30 | 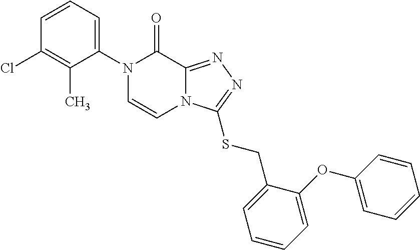 [SMILES: Cc1c(Cl)cccc1-n1ccn2c(SCc3ccccc3Oc3ccccc3)nnc2c1=O] | 7-(3-chloro-2-methylphenyl)-3-(2- phenoxybenzylthio)-[1,2,4]triazolo[4,3- a]pyrazin-8(7H)-one | 1.59% 2.475 | 67.0 |
| 31 | 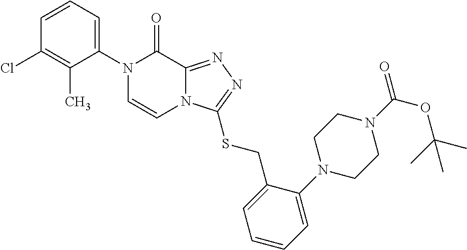 [SMILES: Cc1c(Cl)cccc1-n1ccn2c(SCc3ccccc3N3CCN(C(=O)OC(C)(C)C)CC3)nnc2c1=O] | tert-butyl 4-(2-((7-(3-chloro-2- methylphenyl)-8-oxo-7,8-dihydro- [1,2,4]triazolo[4,3-a]pyrazin-3- ylthio)methyl)phenyl)piperazine-1- carboxylate | 1.75% 2.568 | 1171 |
| 32 | 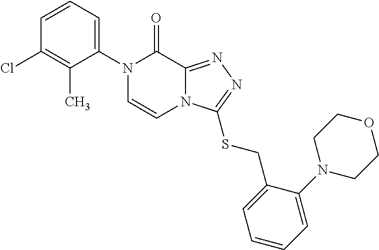 [SMILES: Cc1c(Cl)cccc1-n1ccn2c(SCc3ccccc3N3CCOCC3)nnc2c1=O] | 7-(3-chloro-2-methylphenyl)-3-(2- morpholinobenzylthio)- [1,2,4]triazolo[4,3-a]pyrazin-8(7H)-one | 1.85% 2.468 | 553.3 |
| 33 | 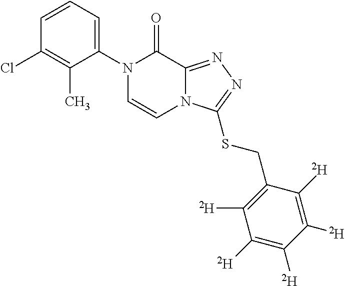 [SMILES: [2H]c1c([2H])c([2H])c(CSc2nnc3c(=O)n(-c4cccc(Cl)c4C)ccn23)c([2H])c1[2H]] | deuterated-7-(3-chloro-2-methylphenyl)- 3-[(phenyl-d5)-methyl)thio]-1,2,4- triazolo[4,3-a]pyrazin-8(7H)-one | 1.69% 2.388 | 85.7 |
| 34 | 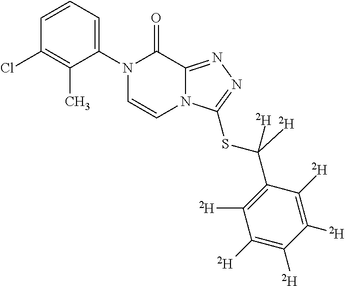 [SMILES: [2H]c1c([2H])c([2H])c(C([2H])([2H])Sc2nnc3c(=O)n(-c4cccc(Cl)c4C)ccn23)c([2H])c1[2H]] | deuterated-7-(3-chloro-2-methylphenyl)- 3-[(phenyl-d5)-methyl-d2))thio]-1,2,4- triazolo[4,3-a]pyrazin-8(7H)-one | 1.88% 2.390 | 148.5 |
| 35 | 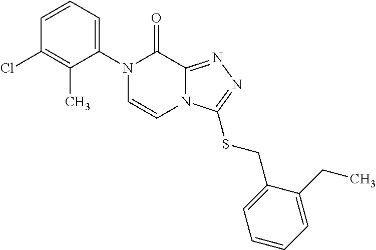 [SMILES: CCc1ccccc1CSc1nnc2c(=O)n(-c3cccc(Cl)c3C)ccn12] | 7-(3-chloro-2-methylphenyl)-3-(2- ethylbenzylthio)-[1,2,4]triazolo[4,3- a]pyrazin-8(7H)-one | 1.59% 2.411 | 213.2 |
| 36 | 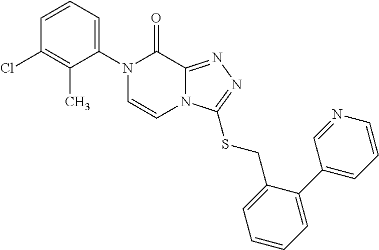 [SMILES: Cc1c(Cl)cccc1-n1ccn2c(SCc3ccccc3-c3cccnc3)nnc2c1=O] | 7-(3-chloro-2-methylphenyl)-3-(2- (pyridin-3-yl)benzytthio)- [1,2,4]triazolo[4,3-a]pyrazin-8(7H)-one | 1.82% 2.460 | 147.2 |
| 37 | 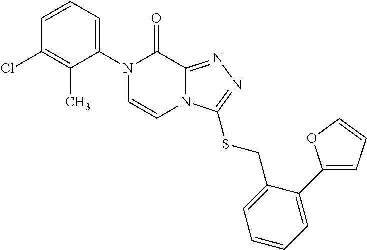 [SMILES: Cc1c(Cl)cccc1-n1ccn2c(SCc3ccccc3-c3ccco3)nnc2c1=O] | 7-(3-chloro-2-methylphenyl)-3-(2- (furan-2-yl)benzylthio)- [1,2,4]triazolo[4,3-a]pyrazin-8(7H)-one | 1.41% 2.449 | 97.4 |
| 38 | 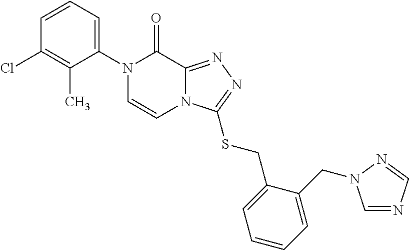 [SMILES: Cc1c(Cl)cccc1-n1ccn2c(SCc3ccccc3Cn3cncn3)nnc2c1=O] | 3-(2-((1H-1,2,4-triazol-1- yl)methyl)benzylthio)-7-(3-chloro-2- methylphenyl)-[1,2,4]triazolo[4,3- a]pyrazin-8(7H)-one | 1.35% 2.464 | 1436 |
| 39 | 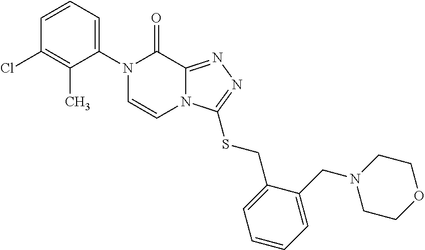 [SMILES: Cc1c(Cl)cccc1-n1ccn2c(SCc3ccccc3CN3CCOCC3)nnc2c1=O] | 7-(3-chloro-2-methylphenyl)-3-(2- (rnorpholinornethyl)benzylthio)- [1,2,4]triazolo[4,3-a]pyrazin-8(7H)-one | 1.12% 2.483 | 635.9 |
| 40 | 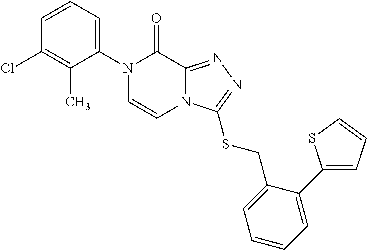 [SMILES: Cc1c(Cl)cccc1-n1ccn2c(SCc3ccccc3-c3cccs3)nnc2c1=O] | 7-(3-chloro-2-methylphenyl)-3-(2- (thiophen-2-yl)benzylthio)- [1,2,4]triazolo[4,3-a]pyrazin-8(7H)-one | 1.19% 2.465 | 45.8 |
| 41 | 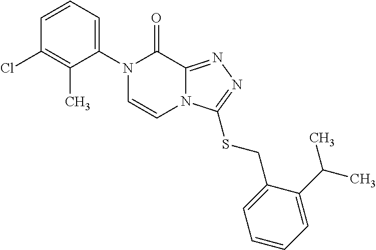 [SMILES: Cc1c(Cl)cccc1-n1ccn2c(SCc3ccccc3C(C)C)nnc2c1=O] | 7-(3-chloro-2-methylphenyl)-3-(2- isopropylbenzylthio)-[1,2,4]triazolo[4,3- a]pyrazin-8(7H)-one | 1.89% 2.425 | 372.3 |
| 42 | 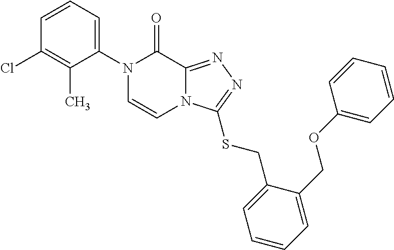 [SMILES: Cc1c(Cl)cccc1-n1ccn2c(SCc3ccccc3COc3ccccc3)nnc2c1=O] | 7-(3-chloro-2-methylphenyl)-3-(2- (phenoxymethyl)benzylthio)- [1,2,4]triazolo[4,3-a]pyrazin-8(7H)-one | 1.34% 2.489 | 170 |
| 43 | 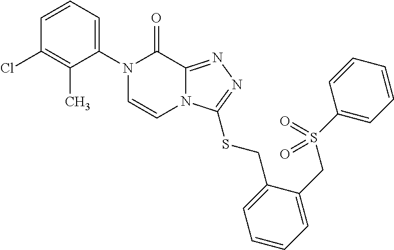 [SMILES: Cc1c(Cl)cccc1-n1ccn2c(SCc3ccccc3CS(=O)(=O)c3ccccc3)nnc2c1=O] | 7-(3-chloro-2-methylphenyl)-3-(2- (phenylsulfonylmethyl)benzylthio)- [1,2,4]triazolo[4,3-a]pyrazin-8(7H)-one | 1.90% 2.538 | 233.4 |
| 44 | 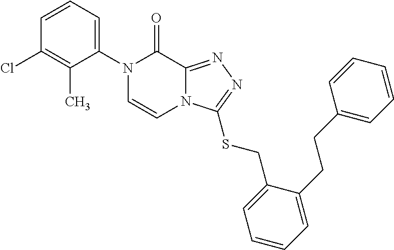 [SMILES: Cc1c(Cl)cccc1-n1ccn2c(SCc3ccccc3CCc3ccccc3)nnc2c1=O] | 7-(3-chloro-2-methylphenyl)-3-(2- phenethylbenzylthio)-[1,2,4jtriazolo[4,3- a]pyrazin-8(7H)-one | 1.53% 2.488 | 552.8 |
| 45 | 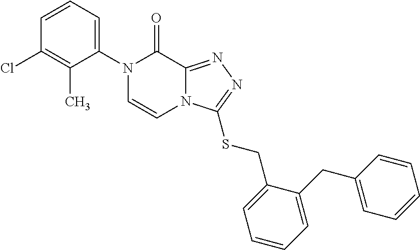 [SMILES: Cc1c(Cl)cccc1-n1ccn2c(SCc3ccccc3Cc3ccccc3)nnc2c1=O] | 3-(2-benzylbenzylthio)-7-(3-chloro-2- methylphenyl)-[1,2,4]triazolo[4,3- a]pyrazin-8(7H)-one | 1.33% 2.473 | 176.2 |
| 46 | 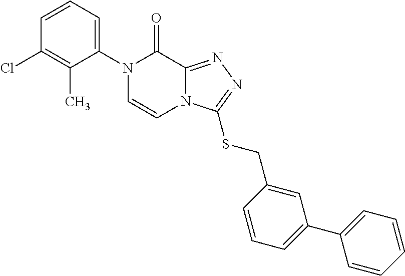 [SMILES: Cc1c(Cl)cccc1-n1ccn2c(SCc3cccc(-c4ccccc4)c3)nnc2c1=O] | 3-(biphenyl-3-ylmethylthio)-7-(3-chloro- 2-methylphenyl)-[1,2,4]triazolo[4,3- a]pyrazin-8(7H)-one | 1.66% 2.459 | 3502 |
| 47 | 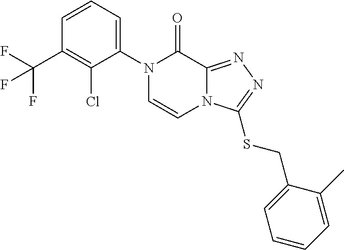 [SMILES: Cc1ccccc1CSc1nnc2c(=O)n(-c3cccc(C(F)(F)F)c3Cl)ccn12] | 7-(2-chloro-3-(trifluorornethyl)phenyl)- 3-(2-methylbenzylthio)- [1,2,4]triazolo[4,3-a]pyrazin-8(7H)-one | 1.44% 2.451 | 41.8 |
| 48 | 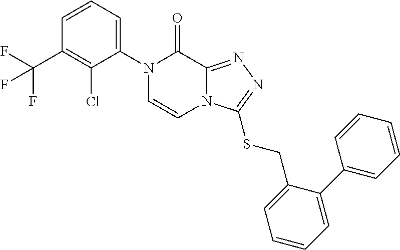 [SMILES: O=c1c2nnc(SCc3ccccc3-c3ccccc3)n2ccn1-c1cccc(C(F)(F)F)c1Cl] | 3-(biphenyl-2-ylmethylthio)-7-(2-chloro- 3-(trifluoromethyl)phenyl)- [1,2,4]triazolo[4,3-a]pyrazin-8(7H)-one | 1.59% 2.513 | 17.3 |
| 49 | 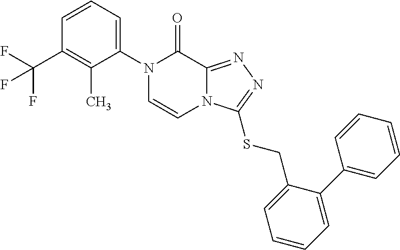 [SMILES: Cc1c(-n2ccn3c(SCc4ccccc4-c4ccccc4)nnc3c2=O)cccc1C(F)(F)F] | 3-(biphenyl-2-ylmethylthio)-7-(2- methyl-3-(trifluoromethyl)phenyl)- [1,2,4]triazolo[4,3-a]pyrazin-8(7H)-one | 1.80% 2.493 | 15.9 |
| 50 | 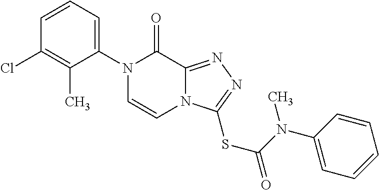 [SMILES: Cc1c(Cl)cccc1-n1ccn2c(SC(=O)N(C)c3ccccc3)nnc2c1=O] | S-7-(3-chloro-2-methylphenyl)-8-oxo- 7,8-dihydro-[1,2,4]triazolo[4,3- a]pyrazin-3-yl methyl(phenyl)carbamothioate | 1.71% 2.426 | 6531 |
| 51 | 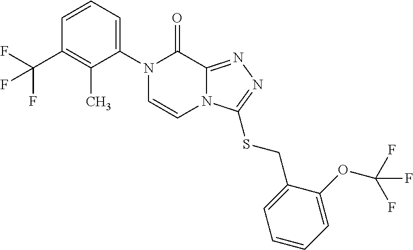 [SMILES: Cc1c(-n2ccn3c(SCc4ccccc4OC(F)(F)F)nnc3c2=O)cccc1C(F)(F)F] | 7-(2-methyl-3-(trifluorornethyl)phenyl)- 3-(2-(trifluoromethoxy)benzylthio)- [1,2,4]triazolo[4,3-a]pyrazin-8(7H)-one | 1.35% 2.501 | 44.2 |
| 52 | 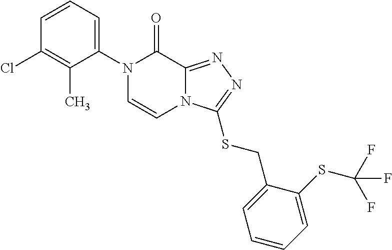 [SMILES: Cc1c(Cl)cccc1-n1ccn2c(SCc3ccccc3SC(F)(F)F)nnc2c1=O] | 7-(3-chloro-2-methylphenyl)-3-(2- (trifluorornethylthio)benzylthio)- [1,2,4]triazolo[4,3-a]pyrazin-8(7H)-one | 1.90% 2.483 | 315.7 |
| 53 | 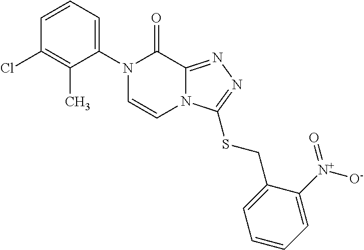 [SMILES: Cc1c(Cl)cccc1-n1ccn2c(SCc3ccccc3[N+](=O)[O-])nnc2c1=O] | 7-(3-chloro-2-methylphenyl)-3-(2- nitrobenzylthio)-[1,2,4]triazolo[4,3- a]pyrazin-8(7H)-one | 1.88% 2.428 | 994.3 |
| 54 | 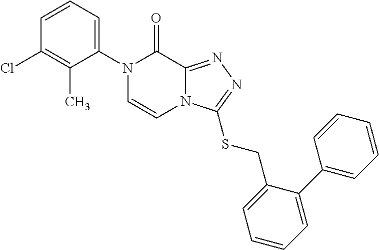 [SMILES: Cc1c(Cl)cccc1-n1ccn2c(SCc3ccccc3-c3ccccc3)nnc2c1=O] | 3-(biphenyl-2-ylmethylthio)-7-(3-chloro- 2-methylphenyl)-[1,2,4]triazolo[4,3- a]pyrazin-8(7H)-one | 1.91% 2.459 | 44.0 |
| 55 | 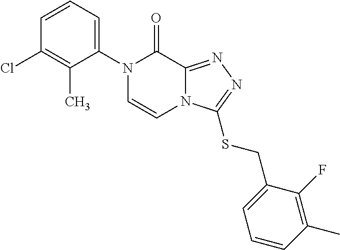 [SMILES: Cc1cccc(CSc2nnc3c(=O)n(-c4cccc(Cl)c4C)ccn23)c1F] | 7-(3-chloro-2-methylphenyl)-3-(2- fluoro-3-methylbenzylthio)- [1,2,4]triazolo[4,3-a]pyrazin-8(7H)-one | 1.84% 2.415 | 432 |
| 56 | 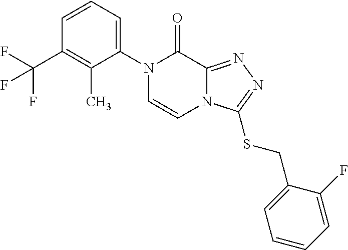 [SMILES: Cc1c(-n2ccn3c(SCc4ccccc4F)nnc3c2=O)cccc1C(F)(F)F] | 3-(2-fluorobenzylthio)-7-(2-methyl-3- (trifluoromethyl)phenyl)- [1,2,4]triazolo[4,3-a]pyrazin-8(7H)-one | 1.53% 2.435 | 166.7 |
| 57 | 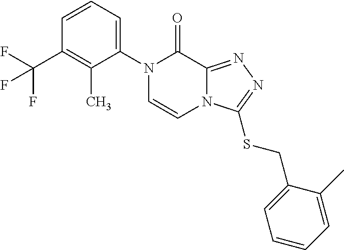 [SMILES: Cc1ccccc1CSc1nnc2c(=O)n(-c3cccc(C(F)(F)F)c3C)ccn12] | 7-(2-methyl-3-(trifluoromethyl)phenyl)- 3-(2-methylbenzylthio)- [1,2,4]triazolo[4,3-a]pyrazin-8(7H)-one | 1.35% 2.431 | 86.4 |
| 58 | 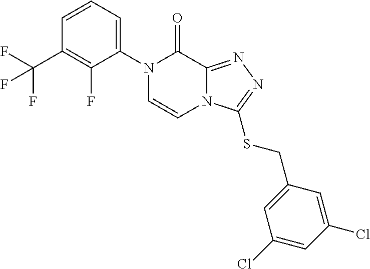 [SMILES: O=c1c2nnc(SCc3cc(Cl)cc(Cl)c3)n2ccn1-c1cccc(C(F)(F)F)c1F] | 3-(3,5-dichlorobenzylthio)-7-(2-fluoro- 3-(trifluoromethyl)phenyl)- [1,2,4]triazolo[4,3-a]pyrazin-8(7H)-one | 1.96% 2.489 | 426.7 |
| 59 | 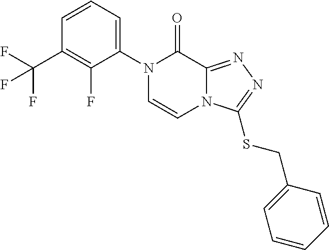 [SMILES: O=c1c2nnc(SCc3ccccc3)n2ccn1-c1cccc(C(F)(F)F)c1F] | 3-(benzylthio)-7-(2-fluoro-3- (trifluoromethyl)phenyl)- [1,2,4]triazolo[4,3-a]pyrazin-8(7H)-one | 1.53% 2.421 | 457.3 |
| 60 | 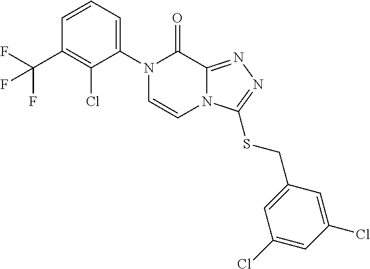 [SMILES: O=c1c2nnc(SCc3cc(Cl)cc(Cl)c3)n2ccn1-c1cccc(C(F)(F)F)c1Cl] | 7-(2-chloro-3-(trifluorornethyl)phenyl)- 3-(3,5-dichlorobenzylthio)- [1,2,4]triazolo[4,3-a]pyrazin-8(7H)-one | 1.78% 2.505 | 91.4 |
| 61 | 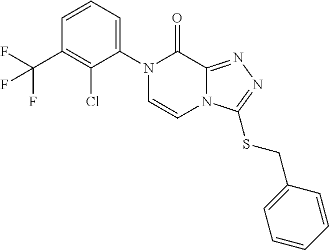 [SMILES: O=c1c2nnc(SCc3ccccc3)n2ccn1-c1cccc(C(F)(F)F)c1Cl] | 3-(benzylthio)-7-(2-chloro-3- (trifluoromethyl)phenyl)- [1,2,4]triazolo[4,3-a]pyrazin-8(7H)-one | 1.73% 2.437 | 44.9 |
| 62 | 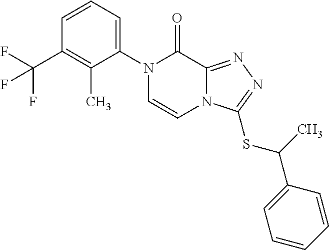 [SMILES: Cc1c(-n2ccn3c(SC(C)c4ccccc4)nnc3c2=O)cccc1C(F)(F)F] | 7-(2-methyl-3-(trifluoromethyl)phenyl)- 3-(1-phenylethylthio)- [1,2,4]triazolo[4,3-a]pyrazin-8(7H)-one | 1.32% 2.431 | 57.5 |
| 63 | 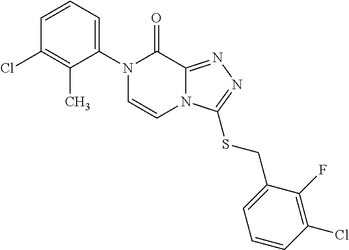 [SMILES: Cc1c(Cl)cccc1-n1ccn2c(SCc3cccc(Cl)c3F)nnc2c1=O] | 3-(3-chloro-2-fluorobenzylthio)-7-(3- chloro-2-methylphenyl)- [1,2,4]triazolo[4,3-a]pyrazin-8(7H)-one | 1.44% 2.435 | 2537 |
| 64 | 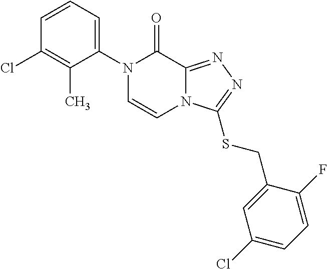 [SMILES: Cc1c(Cl)cccc1-n1ccn2c(SCc3cc(Cl)ccc3F)nnc2c1=O] | 3-(5-chloro-2-fluorobenzylthio)-7-(3- chloro-2-methylphenyl)- [1,2,4]triazolo[4,3-a]pyrazin-8(7H)-one | 1.94% 2.436 | 754.7 |
| 65 | 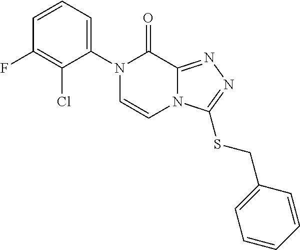 [SMILES: O=c1c2nnc(SCc3ccccc3)n2ccn1-c1cccc(F)c1Cl] | 3-(benzylthio)-7-(2-chloro-3- fluorophenyl)-[1,2,4]triazolo[4,3- a]pyrazin-8(7H)-one | 1.47% 2.387 | 188.5 |
| 66 | 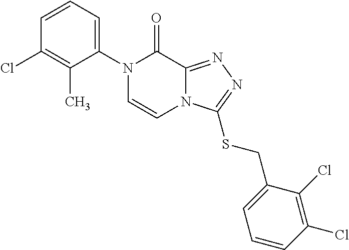 [SMILES: Cc1c(Cl)cccc1-n1ccn2c(SCc3cccc(Cl)c3Cl)nnc2c1=O] | 7-(3-chloro-2-methylphenyl)-3-(2,3- dichlorobenzylthio)-[1,2,4]triazolo[4,3- a]pyrazin-8(7H)-one | 1.27% 2.453 | 324 |
| 67 | 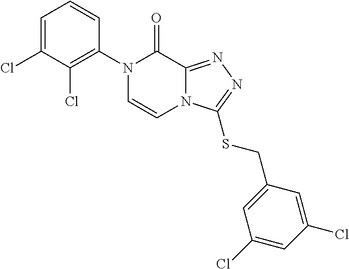 [SMILES: O=c1c2nnc(SCc3cc(Cl)cc(Cl)c3)n2ccn1-c1cccc(Cl)c1Cl] | 3-(3,5-dichlorobenzylthio)-7-(2,3- dichlorophenyl)-[1,2,4]triazolo[4,3- a]pyrazin-8(7H)-one | 1.55% 2.473 | 168 |
| 68 | 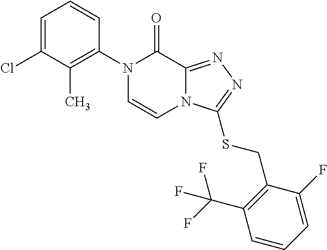 [SMILES: Cc1c(Cl)cccc1-n1ccn2c(SCc3c(F)cccc3C(F)(F)F)nnc2c1=O] | 7-(3-chloro-2-methylphenyl)-3-(2- fluoro-6-(trifluoromethyl)benzylthio)- [1,2,4]triazolo[4,3-a]pyrazin-8(7H)-one | 1.65% 2.469 | 475 |
| 69 | 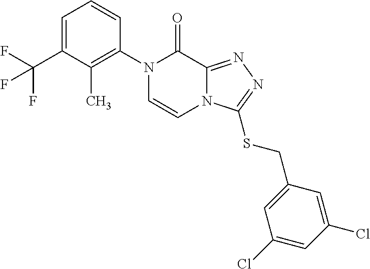 [SMILES: Cc1c(-n2ccn3c(SCc4cc(Cl)cc(Cl)c4)nnc3c2=O)cccc1C(F)(F)F] | 3-(3,5-dichlorobenzylthio)-7-(2-methyl- 3-(trifluoromethyl)phenyl)- [1,2,4]triazolo[4,3-a]pyrazin-8(7H)-one | 1.34% 2.485 | 187.7 |
| 70 | 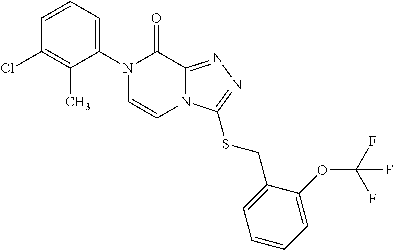 [SMILES: Cc1c(Cl)cccc1-n1ccn2c(SCc3ccccc3OC(F)(F)F)nnc2c1=O] | 7-(3-chloro-2-methylphenyl)-3-(2- (trifluoromethoxy)benzylthio)- [1,2,4]triazolo[4,3-a]pyrazin-8(7H)-one | 1.82% 2.467 | 111.7 |
| 71 | 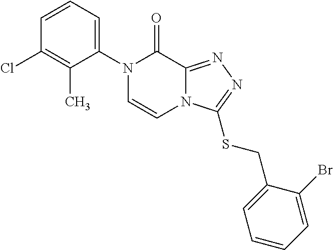 [SMILES: Cc1c(Cl)cccc1-n1ccn2c(SCc3ccccc3Br)nnc2c1=O] | 3-(2-bromobenzylthio)-7-(3-chloro-2- methylphenyl)-[1,2,4]triazolo[4,3- a]pyrazin-8(7H)-one | 1.80% 2.463 | 142.1 |
| 72 | 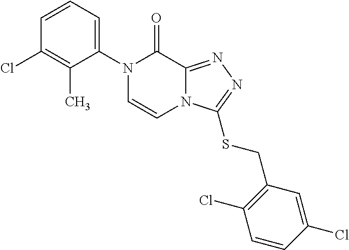 [SMILES: Cc1c(Cl)cccc1-n1ccn2c(SCc3cc(Cl)ccc3Cl)nnc2c1=O] | 7-(3-chloro-2-methylphenyl)-3-(2,5- dichlorobenzylthio)-[1,2,4]triazolo[4,3- a]pyrazin-8(7H)-one | 1.99% 2.453 | 3155 |
| 73 | 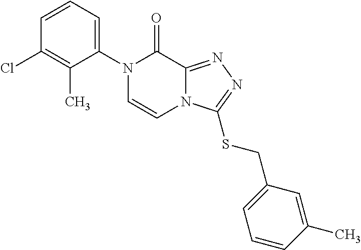 [SMILES: Cc1cccc(CSc2nnc3c(=O)n(-c4cccc(Cl)c4C)ccn23)c1] | 7-(3-chloro-2-methylphenyl)-3-(3- methylbenzylthio)-[1,2,4]triazolo[4,3- a]pyrazin-8(7H)-one | 1.72% 2.397 | 438.7 |
| 74 | 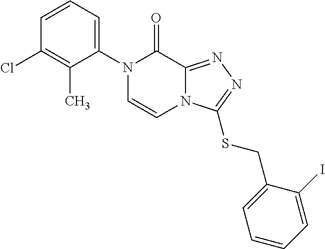 [SMILES: Cc1c(Cl)cccc1-n1ccn2c(SCc3ccccc3I)nnc2c1=O] | 7-(3-chloro-2-methylphenyl)-3-(2- iodobenzylthio)-[1,2,4]triazolo[4,3- a]pyrazin-8(7H)-one | 1.48% 2.509 | 263.5 |
| 75 | 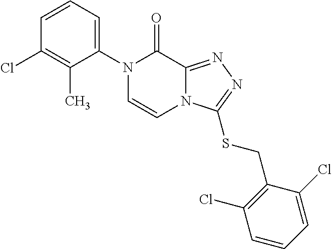 [SMILES: Cc1c(Cl)cccc1-n1ccn2c(SCc3c(Cl)cccc3Cl)nnc2c1=O] | 7-(3-chloro-2-methylphenyl)-3-(2,6- dichlorobenzylthio)-[1,2,4]triazolo[4,3- a]pyrazin-8(7H)-one | 1.91% 2.452 | 279.7 |
| 76 | 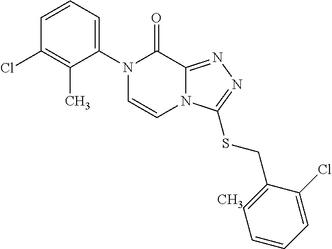 | 7-(3-chloro-2-methylphenyl)-3-(2,6- dimethylbenzylthio)-[1,2,4]triazolo[4,3- a]pyrazin-8(7H)-one | 1.41% 2.411 | 244.9 |
| 77 | 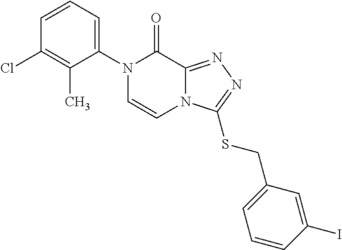 [SMILES: Cc1c(Cl)cccc1-n1ccn2c(SCc3cccc(I)c3)nnc2c1=O] | 7-(3-chloro-2-methylphenyl)-3-(3- iodobenzylthio)-[1,2,4]triazolo[4,3- a]pyrazin-8(7H)-one | 1.15% 2.509 | 461.9 |
| 78 | 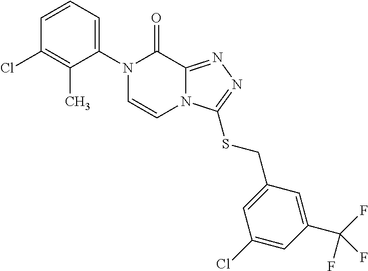 [SMILES: Cc1c(Cl)cccc1-n1ccn2c(SCc3cc(Cl)cc(C(F)(F)F)c3)nnc2c1=O] | 7-(3-chloro-2-methylphenyl)-3-(3- chloro-5-(trifluoromethyl)benzylthio)- [1,2,4]triazolo[4,3-a]pyrazin-8(7H)-one | 1.56% 2.485 | 597.1 |
| 79 | 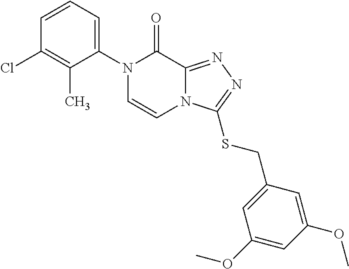 [SMILES: COc1cc(CSc2nnc3c(=O)n(-c4cccc(Cl)c4C)ccn23)cc(OC)c1] | 7-(3-chloro-2-methylphenyl)-3-(3,5- dimethoxybenzylthio)- [1,2,4]triazolo[4,3-a]pyrazin-8(7H)-one | 1.61% 2.443 | 1332 |
| 80 | 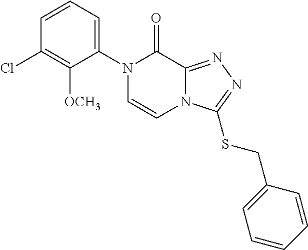 [SMILES: COc1c(Cl)cccc1-n1ccn2c(SCc3ccccc3)nnc2c1=O] | 3-(benzylthio)-7-(3-chloro-2- methoxyphenyl)-[1,2,4]triazolo[4,3- a]pyrazin-8(7H)-one | 1.40% 2.399 | 532.9 |
| 81 | 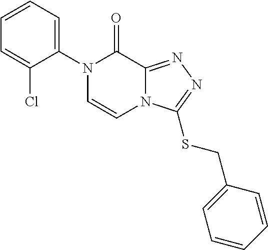 [SMILES: O=c1c2nnc(SCc3ccccc3)n2ccn1-c1ccccc1Cl] | 3-(benzylthio)-7-(2-chlorophenyl)- [1,2,4]triazolo[4,3-a]pyrazin-8(7H)-one | 1.49% 2.369 | 946.3 |
| 82 | 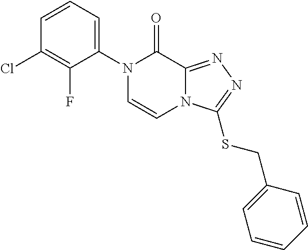 [SMILES: O=c1c2nnc(SCc3ccccc3)n2ccn1-c1cccc(Cl)c1F] | 3-(benzylthio)-7-(3-chloro-2- fluorophenyl)-[1,2,4]triazolo[4,3- a]pyrazin-8(7H)-one | 1.61% 2.387 | 374.2 |
| 83 | 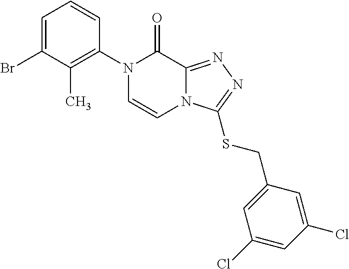 [SMILES: Cc1c(Br)cccc1-n1ccn2c(SCc3cc(Cl)cc(Cl)c3)nnc2c1=O] | 7-(3-brorno-2-methylphenyl)-3-(3,5- dichlorobenzylthio)-[1,2,4]triazolo[4,3- a]pyrazin-8(7H)-one | 1.47% 2.497 | 855.4 |
| 84 | 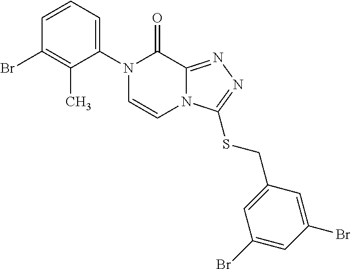 [SMILES: Cc1c(Br)cccc1-n1ccn2c(SCc3cc(Br)cc(Br)c3)nnc2c1=O] | 7-(3-chloro-2-methylphenyl)-3-(3,5- dibromobenzylthio)-[1,2,4]triazolo[4,3- a]pyrazin-8(7H)-one | 1.57% 2.541 | 522.3 |
| 85 | 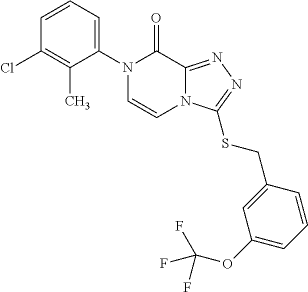 [SMILES: Cc1c(Cl)cccc1-n1ccn2c(SCc3cccc(OC(F)(F)F)c3)nnc2c1=O] | 7-(3-chloro-2-methylphenyI)-3-(3- (trifluoromethoxy)benzylthio)- [1,2,4]triazolo[4,3-a]pyrazin-8(7H)-one | 1.91% 2.467 | 502.6 |
| 86 | 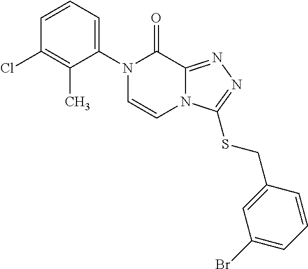 [SMILES: Cc1c(Cl)cccc1-n1ccn2c(SCc3cccc(Br)c3)nnc2c1=O] | 3-(3-bromobenzylthio)-7-(3-chloro-2- methylphenyl)-[1,2,4]triazolo[4,3- a]pyrazin-8(7H)-one | 1.93% 2.462 | 445.7 |
| 87 | 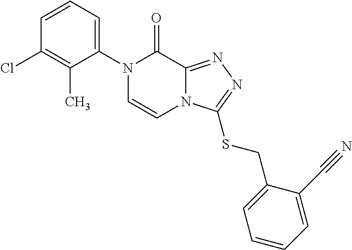 [SMILES: Cc1c(Cl)cccc1-n1ccn2c(SCc3ccccc3C#N)nnc2c1=O] | 2((7-(3-chloro-2-methylphenyl)-8-oxo- 7,8-dihydro-[1,2,4]triazolo[4,3- a]pyrazin-3-ylthio)methyl)benzonitrile | 1.95% 2.408 | 434.4 |
| 88 | 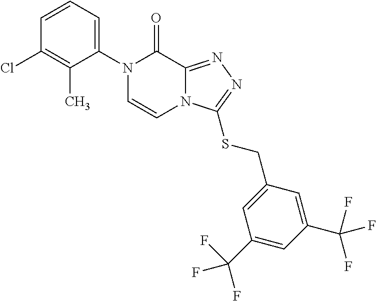 [SMILES: Cc1c(Cl)cccc1-n1ccn2c(SCc3cc(C(F)(F)F)cc(C(F)(F)F)c3)nnc2c1=O] | 3-(3,5-bis(trifluoromethyl)benzylthio)-7- (3-chloro-2-methylphenyl)- [1,2,4]triazolo[4,3-a]pyrazin-8(7H)-one | 1.92% 2.519 | 8372 |
| 89 | 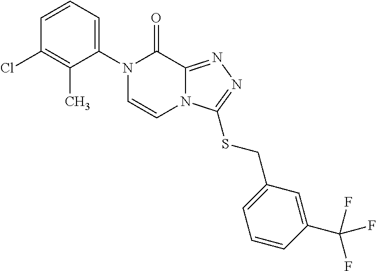 [SMILES: Cc1c(Cl)cccc1-n1ccn2c(SCc3cccc(C(F)(F)F)c3)nnc2c1=O] | 7-(3-chloro-2-methylphenyl)-3-(3- (trifluoromethyl)benzylthio)- [1,2,4]triazolo[4,3-a]pyrazin-8(7H)-one | 1.96% 2.451 | 580.6 |
| 90 | 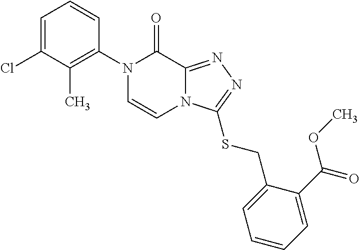 [SMILES: COC(=O)c1ccccc1CSc1nnc2c(=O)n(-c3cccc(Cl)c3C)ccn12] | methyl 2-((7-(3-chloro-2-methylphenyl)- 8-oxo-7,8-dihydro-[1,2,4]triazolo[4,3- a]pyrazin-3-ylthio)methyl)benzoate | 1.84% 2.441 | 643.3 |
| 91 | 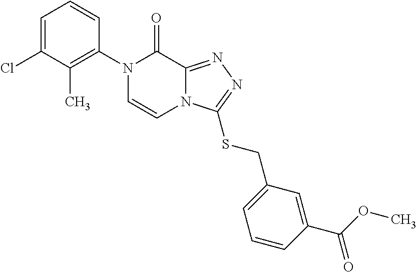 [SMILES: COC(=O)c1cccc(CSc2nnc3c(=O)n(-c4cccc(Cl)c4C)ccn23)c1] | methyl 3-((7-(3-chloro-2-methylphenyl)- 8-oxo-7,8-dihydro-[1,2,4]triazolo[4,3- a]pyrazin-3-ylthio)methyl)benzoate | 1.92% 2.441 | 3254 |
| 92 | 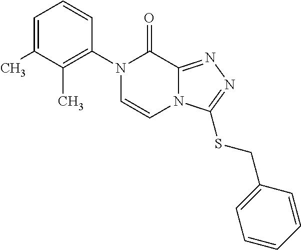 [SMILES: Cc1cccc(-n2ccn3c(SCc4ccccc4)nnc3c2=O)c1C] | 3-(benzylthio)-7-(2,3-dimethylphenyl)- [1,2,4]triazolo[4,3-a]pyrazin-8(7H)-one | 1.16% 2.363 | 208.8 |
| 93 | 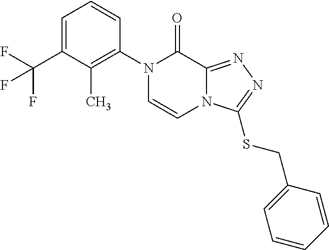 [SMILES: Cc1c(-n2ccn3c(SCc4ccccc4)nnc3c2=O)cccc1C(F)(F)F] | 3-(benzylthio)-7-(2-methyl-3- (trifluoromethyl)phenyl)- [1,2,4]triazolo[4,3-a]pyrazin-8(7H)-one | 1.39% 2.417 | 38.2 |
| 94 | 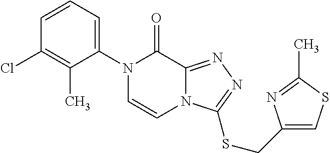 [SMILES: Cc1nc(CSc2nnc3c(=O)n(-c4cccc(Cl)c4C)ccn23)cs1] | 7-(3-chloro-2-methylphenyl)-3((2- methylthiazol-4-yl)methylthio)- [1,2,4]triazolo[4,3-a]pyrazin-8(7H)-one | 1.76% 2.404 | 2556 |
| 95 | 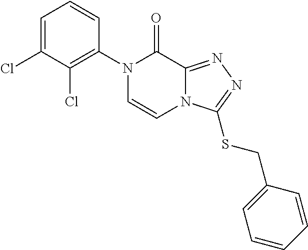 [SMILES: O=c1c2nnc(SCc3ccccc3)n2ccn1-c1cccc(Cl)c1Cl] | 3-(benzylthio)-7-(2,3-dichlorophenyl)- [1,2,4]triazolo[4,3-a]pyrazin-8(7H)-one | 1.23% 2.404 | 109.5 |
| 96 | 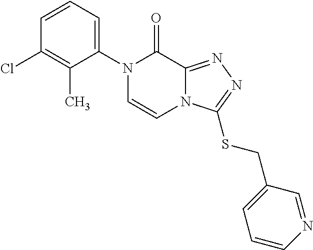 [SMILES: Cc1c(Cl)cccc1-n1ccn2c(SCc3cccnc3)nnc2c1=O] | 7-(3-chloro-2-methylphenyl)-3-(pyridin- 3-ylmethylthio)-[1,2,4]triazolo[4,3- a]pyrazin-8(7H)-one | 1.71% 2.384 | 4221 |
| 97 | 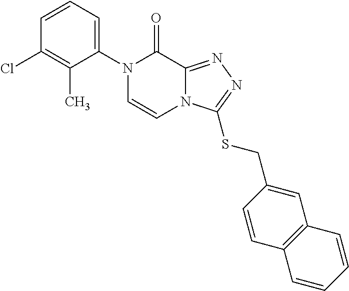 [SMILES: Cc1c(Cl)cccc1-n1ccn2c(SCc3ccc4ccccc4c3)nnc2c1=O] | 7-(3-chloro-2-methylphenyl)-3- (naphthalen-2-ylmethylthio)- [1,2,4]triazolo[4,3-a]pyrazin-8(7H)-one | 1.48% 2.433 | 2306 |
| 98 | 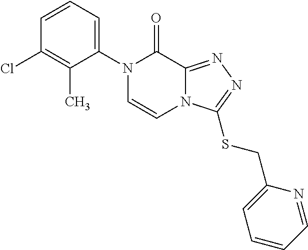 [SMILES: Cc1c(Cl)cccc1-n1ccn2c(SCc3ccccn3)nnc2c1=O] | 7-(3-chloro-2-methylphenyl)-3-(pyridin- 2-ylmethylthio)-[1,2,4]thazolo[4,3- a]pyrazin-8(7H)-one | 1.87% 2.384 | 2339 |
| 99 | 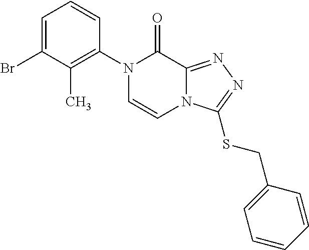 [SMILES: Cc1c(Br)cccc1-n1ccn2c(SCc3ccccc3)nnc2c1=O] | 3-(benzylthio)-7-(3-bromo-2- methylphenyl)-[1,2,4]-triazolo[4,3- a]pyrazin-8(7H)-one | 1.88% 2.428 | 250.3 |
| 100 | 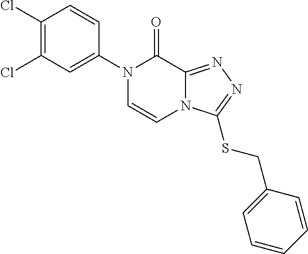 [SMILES: O=c1c2nnc(SCc3ccccc3)n2ccn1-c1ccc(Cl)c(Cl)c1] | 3-(benzylthio)-7-(3,4-dichlorophenyl)- [1,2,4]triazolo[4,3-a]pyrazin-8(7H)-one | 1.88% 2.403 | 3923 |
| 101 | 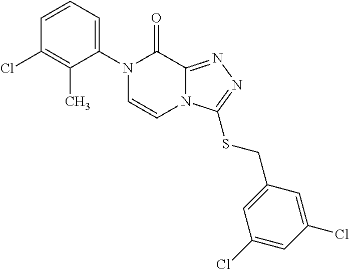 [SMILES: Cc1c(Cl)cccc1-n1ccn2c(SCc3cc(Cl)cc(Cl)c3)nnc2c1=O] | 7-(3-chloro-2-methylphenyl)-3-(3,5- dichlorobenzylthio)-[1,2,4]triazolo[4,3- a]pyrazin-8(7H)-one | 1.56% 2.452 | 167.1 |
| 102 | 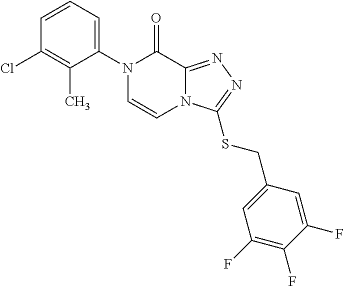 [SMILES: Cc1c(Cl)cccc1-n1ccn2c(SCc3cc(F)c(F)c(F)c3)nnc2c1=O] | 7-(3-chloro-2-methylphenyl)-3-(3,4,5- trifluorobenzylthio)-[1,2,4]triazolo[4,3- a]pyrazin-8(7H)-one | 1.95% 2.437 | 924.9 |
| 103 | 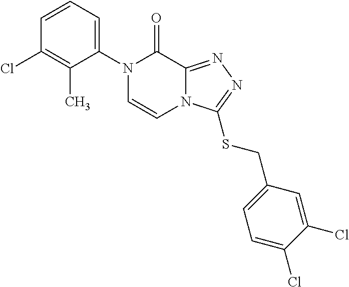 [SMILES: Cc1c(Cl)cccc1-n1ccn2c(SCc3ccc(Cl)c(Cl)c3)nnc2c1=O] | 7-(3-chloro-2-methylphenyl)-3-(3,4- dichlorobenzylthio)-[1,2,4]triazolo[4,3- a]pyrazin-8(7H)-one | 1.96% 2.453 | 1304 |
| 104 | 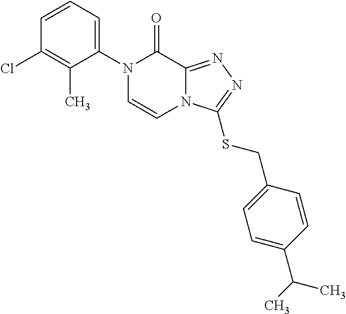 [SMILES: Cc1c(Cl)cccc1-n1ccn2c(SCc3ccc(C(C)C)cc3)nnc2c1=O] | 7-(3-chloro-2-methylphenyl)-3-(4- isopropylbenzylthio)-[1,2,4]triazolo[4,3- a]pyrazin-8(7H)-one | 1.84% 2.425 | 2736 |
| 105 | 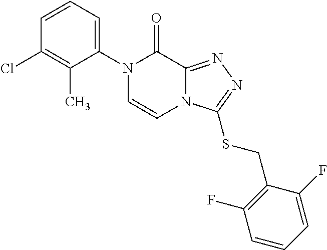 [SMILES: Cc1c(Cl)cccc1-n1ccn2c(SCc3c(F)cccc3F)nnc2c1=O] | 7-(3-chloro-2-methylphenyl)-3-(2,6- difluorobenzylthio)-[1,2,4]triazolo[4,3- a]pyrazin-8(7H)-one | 1.97% 2.419 | 389.2 |
| 106 | 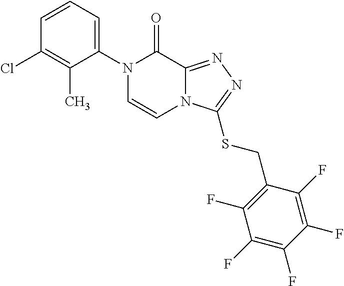 [SMILES: Cc1c(Cl)cccc1-n1ccn2c(SCc3c(F)c(F)c(F)c(F)c3F)nnc2c1=O] | 7-(3-chloro-2-methylphenyl)-3- (perfluorobenzylthio)- [1,2,4]triazolo[4,3-a]pyrazin-8(7H)-one | 1.94% 2.473 | 386.7 |
| 107 | 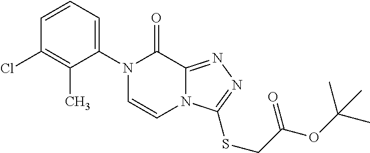 [SMILES: Cc1c(Cl)cccc1-n1ccn2c(SCC(=O)OC(C)(C)C)nnc2c1=O] | tert-butyl 2-(7-(3-chloro-2- methylphenyl)-8-oxo-7,8-dihydro- [1,2,4]triazolo[4,3-a]pyrazin-3- ylthio)acetate | 1.86% 2.407 | 1821 |
| 108 | 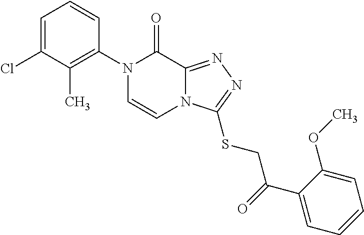 [SMILES: COc1ccccc1C(=O)CSc1nnc2c(=O)n(-c3cccc(Cl)c3C)ccn12] | 7-(3-chloro-2-methylphenyl)-3-(2-(2- methoxyphenyl)-2-oxoethylthio)- [1,2,4]triazolo[4,3-a]pyrazin-8(7H)-one | 1.58% 2.441 | 507.3 |
| 109 | 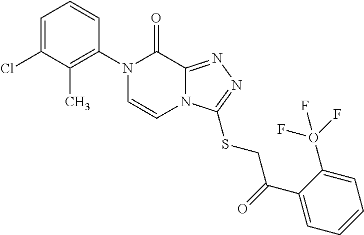 | 7-(3-chloro-2-methylphenyl)-3-(2-oxo- 2-(2-(trifluoromethyl)phenyl)ethylthio)- [1,2,4]-triazolo[4,3-a]pyrazin-8(7H)-one | 1.38% 2.479 | 447.9 |
| 110 | 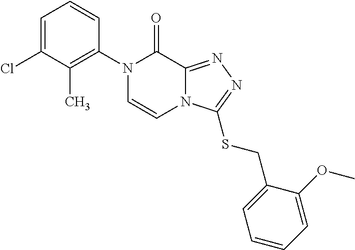 [SMILES: COc1ccccc1CSc1nnc2c(=O)n(-c3cccc(Cl)c3C)ccn12] | 7-(3-chloro-2-methylphenyl)-3-(2- methoxybenzylthio)-[1,2,4]triazolo[4,3- a]pyrazin-8(7H)-one | 1.78% 2.413 | 544.3 |
| 111 | 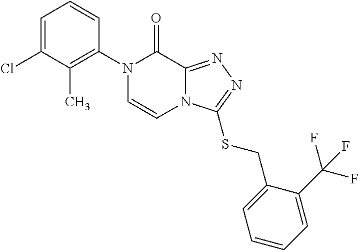 [SMILES: Cc1c(Cl)cccc1-n1ccn2c(SCc3ccccc3C(F)(F)F)nnc2c1=O] | 7-(3-chloro-2-methylphenyl)-3-(2- (trifluoromethyl)benzylthio)- [1,2,4]triazolo[4,3-a]pyrazin-8(7H)-one | 1.95% 2.451 | 515.4 |
| 112 | 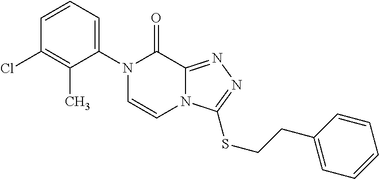 [SMILES: Cc1c(Cl)cccc1-n1ccn2c(SCCc3ccccc3)nnc2c1=O] | 7-(3-chloro-2-methylphenyl)-3- (phenethylthio)-[1,2,4]triazolo[4,3- a]pyrazin-8(7H)-one | 1.38% 2.397 | 660.1 |
| 113 | 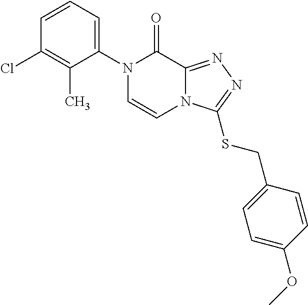 [SMILES: COc1ccc(CSc2nnc3c(=O)n(-c4cccc(Cl)c4C)ccn23)cc1] | 7-(3-chloro-2-methylphenyl)-3-(4- methoxybenzylthio)-[1,2,4]triazolo[4,3- a]pyrazin-8(7H)-one | 1.88% 2.413 | 5651 |
| 114 | 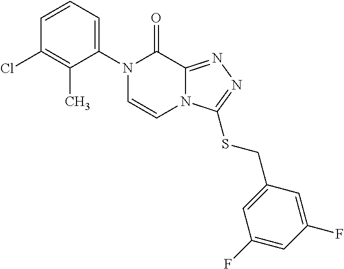 [SMILES: Cc1c(Cl)cccc1-n1ccn2c(SCc3cc(F)cc(F)c3)nnc2c1=O] | 7-(3-chloro-2-methylphenyl)-3-(3,5- difluorobenzylthio)-[1,2,4]triazolo[4,3- a]pyrazin-8(7H)-one | 1.94% 2.419 | 697.1 |
| 115 | 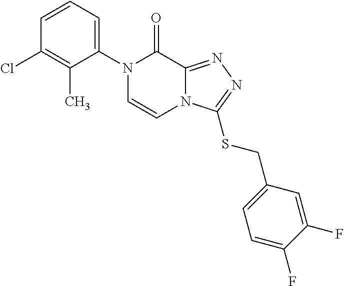 [SMILES: Cc1c(Cl)cccc1-n1ccn2c(SCc3ccc(F)c(F)c3)nnc2c1=O] | 7-(3-chloro-2-methylphenyl)-3-(3,4- difluorobenzylthio)-[1,2,4]triazolo[4,3- a]pyrazin-8(7H)-one | 1.94% 2.419 | 893.4 |

### Example 6

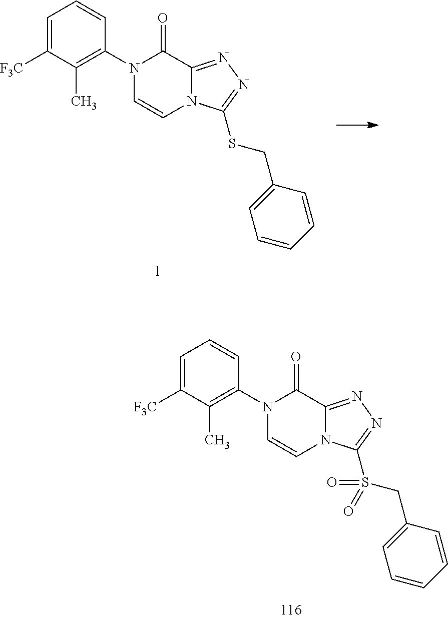 [SMILES: Cc1c(-n2ccn3c(SCc4ccccc4)nnc3c2=O)cccc1C(F)(F)F ; Cc1c(-n2ccn3c(S(=O)(=O)Cc4ccccc4)nnc3c2=O)cccc1C(F)(F)F]

Into a 1-neck round-bottom flask was added compound 1 (0.037 g, 0.089 mmol) and m-chloroperbenzoic acid (0.0299 g, 0.133 mmol) in methylene chloride (1.0 mL). The solution was stirred at room temperature for 17 hours. The compound was purified on a 2000 micron preparative plate using 5% acetone in methylene chloride to yield compound 116 (0.023 g, 59%) as a white solid. LC-MS: MW [MH⁺]=449.

By essentially the same procedure set forth in Example 6, the compounds given in Column 2 of Table 4, i.e., compounds 116-165, were prepared from the appropriate thioether precursors.

TABLE 4

| | | | 1. % Yield | |
|---|---|---|---|---|
| | | | 2. LC-MS: | P2X₇ Ca Flux |
| Example | Structural Formula | Chemical Name | MW[MH⁺] | hu IC₅₀ nM |
| 116 | 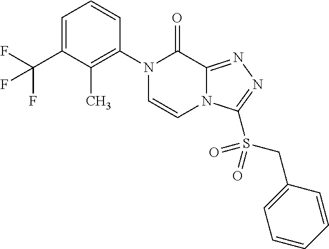 [SMILES: Cc1c(-n2ccn3c(S(=O)(=O)Cc4ccccc4)nnc3c2=O)cccc1C(F)(F)F] | 3-(benzylsulfonyl)-7-(2-methyl-3- (trifluoromethyl)phenyl)- [1,2,4]triazolo[4,3-a]pyrazin-8(7H- one | 1. 59% 2. 449 | 29.0 |
| 117 | 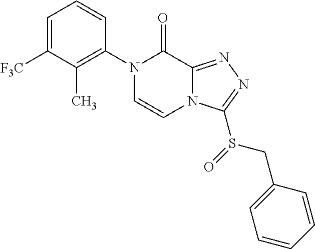 [SMILES: Cc1c(-n2ccn3c(S(=O)Cc4ccccc4)nnc3c2=O)cccc1C(F)(F)F] | 3-(benzylsulfinyl)-7-(2-methyl-3- (trifluoromethyl)phenyl)- [1,2,4]triazolo[4,3-a]pyrazin-8(7H)- one | 1. 77% 2. 433 | 76.5 |
| 118 | 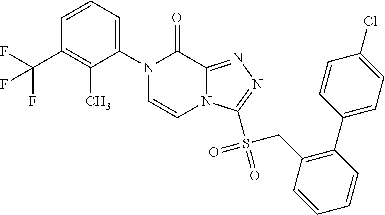 [SMILES: Cc1c(-n2ccn3c(S(=O)(=O)Cc4ccccc4-c4ccc(Cl)cc4)nnc3c2=O)cccc1C(F)(F)F] | 3-((4′-chlorobiphenyl-2- yl)methylsulfonyl)-7-(2-methyl-3- (trifluoromethyl)phenyl)- [1,2,4]triazolo[4,3-a]pyrazin-8(7H)- one | 1. 83% 2. 559 | 6.9 |
| 119 | 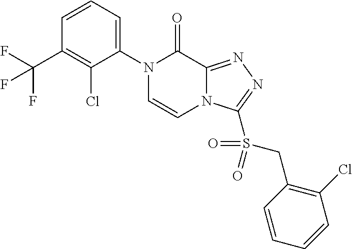 [SMILES: O=c1c2nnc(S(=O)(=O)Cc3ccccc3Cl)n2ccn1-c1cccc(C(F)(F)F)c1Cl] | 7-(2-chloro-3- (trifluoromethyl)phenyl)-3-(2- chlorobenzylsulfonyl)- [1,2,4]triazolo[4,3-a]pyrazin-8(7H)- one | 1. 74% 2. 503 | 11.0 |
| 120 | 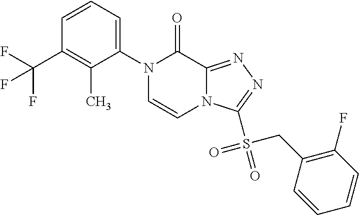 [SMILES: Cc1c(-n2ccn3c(S(=O)(=O)Cc4ccccc4F)nnc3c2=O)cccc1C(F)(F)F] | 3-(2-fluorobenzylsulfonyl)-7-(2- methyl-3-(trifluoromethyl)phenyl)- [1,2,4]triazolo[4,3-a]pyrazin-8(7H)- one | 1. 63% 2. 467 | 11.8 |
| 121 | 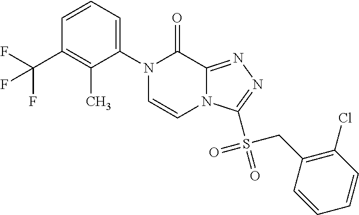 [SMILES: Cc1c(-n2ccn3c(S(=O)(=O)Cc4ccccc4Cl)nnc3c2=O)cccc1C(F)(F)F] | 3-(2-chlorobenzylsulfonyl)-7-(2- methyl-3-(trifluoromethyl)phenyl)- [1,2,4]triazolo[4,3-a]pyrazin-8(7H)- one | 1. 28% 2. 483 | 11.9 |
| 122 | 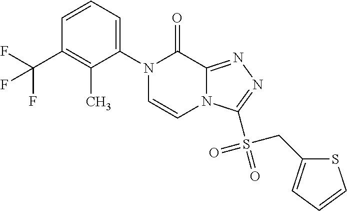 [SMILES: Cc1c(-n2ccn3c(S(=O)(=O)Cc4cccs4)nnc3c2=O)cccc1C(F)(F)F] | 7-(2-methyl-3- (trifluoromethyl)phenyl)-3 -(thiophen- 2-ylmethylsulfonyl)- [1,2,4]triazolo[4,3-a]pyrazin-8(7H)- one | 1. 73% 2. 455 | 12.3 |
| 123 | 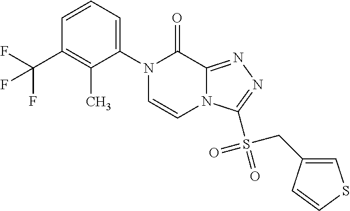 [SMILES: Cc1c(-n2ccn3c(S(=O)(=O)Cc4ccsc4)nnc3c2=O)cccc1C(F)(F)F] | 7-(2-methyl-3- (trifluoromethyl)phenyl)-3-(thiophen- 3-ylmethylsulfonyl)- [1,2,4]triazolo[4,3-a]pyrazin-8(7H)- one | 1. 54% 2. 455 | 12.4 |
| 124 | 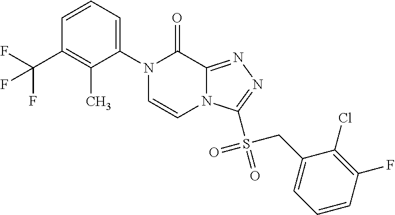 [SMILES: Cc1c(-n2ccn3c(S(=O)(=O)Cc4cccc(F)c4Cl)nnc3c2=O)cccc1C(F)(F)F] | 3-(2-chloro-3-fluorobenzylsulfonyl)- 7-(2-methyl-3- (trifluoromethyl)phenyl)- [1,2,4]triazolo[4,3-a]pyrazin-8(7H)- one | 1. 22% 2. 501 | 14.6 |
| 125 | 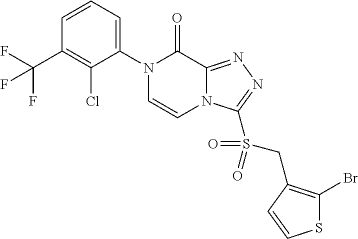 [SMILES: O=c1c2nnc(S(=O)(=O)Cc3ccsc3Br)n2ccn1-c1cccc(C(F)(F)F)c1Cl] | 3-((2-bromothiophen-3- yl)methylsulfonyl)-7-(2-methyl-3- (trifluoromethyl)phenyl)- [1,2,4]triazolo[4,3-a]pyrazin-8(7H)- one | 1. 84% 2. 533 | 15.5 |
| 126 | 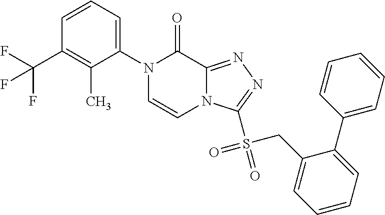 [SMILES: Cc1c(-n2ccn3c(S(=O)(=O)Cc4ccccc4-c4ccccc4)nnc3c2=O)cccc1C(F)(F)F] | 3-(biphenyl-2-ylmethylthio)-7-(2- methyl-3-(trifluoromethyl)phenyl)- [1,2,4]triazolo[4,3-a]pyrazin-8(7H)- one | 1. 49% 2. 525 | 15.9 |
| 127 | 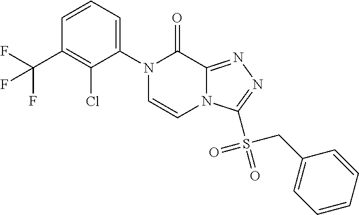 [SMILES: O=c1c2nnc(S(=O)(=O)Cc3ccccc3)n2ccn1-c1cccc(C(F)(F)F)c1Cl] | 3-(benzylsulfonyl)-7-(2-chloro-3- (trifluoromethyl)phenyl)- [1,2,4]triazolo[4,3-a]pyrazin-8(7H)- one | 1. 85% 2. 469 | 16.3 |
| 128 | 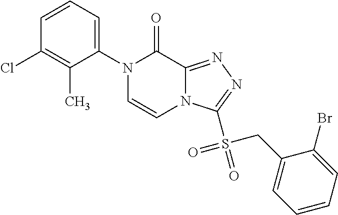 [SMILES: Cc1c(Cl)cccc1-n1ccn2c(S(=O)(=O)Cc3ccccc3Br)nnc2c1=O] | 3-(2-bromobenzylsulfonyl)-7-(3- chloro-2-methylphenyl)- [1,2,4]triazolo[4,3-a]pyrazin-8(7H)- one | 1. 78% 2. 495 | 16.9 |
| 129 | 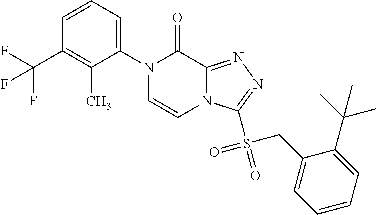 [SMILES: Cc1c(-n2ccn3c(S(=O)(=O)Cc4ccccc4C(C)(C)C)nnc3c2=O)cccc1C(F)(F)F] | 3-(2-tert-butylbenzylsulfonyl)-7-(2- methyl-3-(trifluoromethyl)phenyl)- [1,2,4]triazolo[4,3-a]pyrazin-8(7H)- one | 1. 72% 2. 504 | 17.0 |
| 130 | 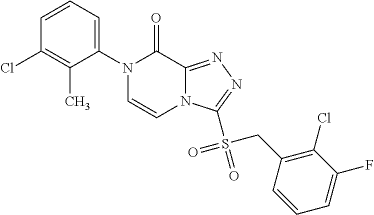 [SMILES: Cc1c(Cl)cccc1-n1ccn2c(S(=O)(=O)Cc3cccc(F)c3Cl)nnc2c1=O] | 7-(3-chloro-2-methylphenyl)-3-(2- chloro-3-fluorobenzylsulfonyl)- [1,2,4]triazolo[4,3-a]pyrazin-8(7H)- one | 1. 27% 2. 467 | 17.3 |
| 131 | 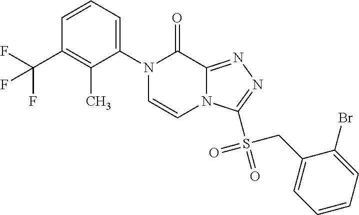 [SMILES: Cc1c(-n2ccn3c(S(=O)(=O)Cc4ccccc4Br)nnc3c2=O)cccc1C(F)(F)F] | 3-(2-bromobenzylsulfonyl)-7-(2- methyl-3-(trifluoromethyl)phenyl)- [1,2,4]triazolo[4,3-a]pyrazin-8(7H)- one | 1. 37% 2. 529 | 17.7 |
| 132 | 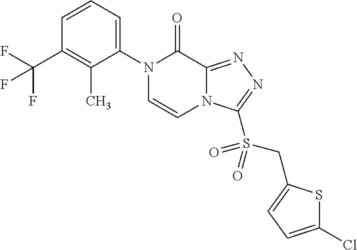 [SMILES: Cc1c(-n2ccn3c(S(=O)(=O)Cc4ccc(Cl)s4)nnc3c2=O)cccc1C(F)(F)F] | 3-((5-chlorothiophen-2- yl)methylsulfonyl)-7-(2-methyl-3- (trifluoromethyl)phenyl)- [1,2,4]triazolo[4,3-a]pyrazin-8(7H)- one | 1. 78% 2. 489 | 17.9 |
| 133 | 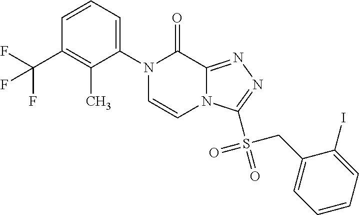 [SMILES: Cc1c(-n2ccn3c(S(=O)(=O)Cc4ccccc4I)nnc3c2=O)cccc1C(F)(F)F] | 3-(2-iodobenzylsulfonyl)-7-(2-methyl- 3-(trifluoromethyl)phenyl)- [1,2,4]triazolo[4,3-a]pyrazin-8(7H)- one | 1. 54% 2. 575 | 21.4 |
| 134 | 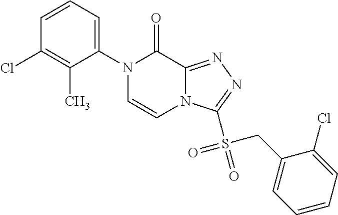 [SMILES: Cc1c(Cl)cccc1-n1ccn2c(S(=O)(=O)Cc3ccccc3Cl)nnc2c1=O] | 7-(3-chloro-2-methylphenyl)-3-(2- chlorobenzylsulfonyl)- [1,2,4]triazolo[4,3-a]pyrazin-8(7H)- one | 1. 62% 2. 449 | 23.2 |
| 135 | 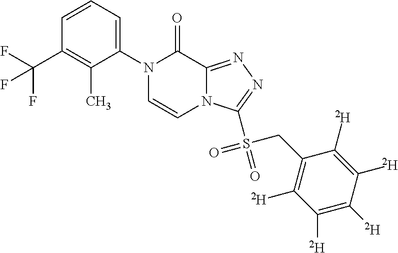 [SMILES: [2H]c1c([2H])c([2H])c(CS(=O)(=O)c2nnc3c(=O)n(-c4cccc(C(F)(F)F)c4C)ccn23)c([2H])c1[2H]] | deuterated-7-[2-methyl-3- (trifluoromethyl)phenyl]-3-[(phenyl- (d5)-methyl)sulfonyl]-1,2,4- triazolo[4,3-a]pyrazin-8(7H)-one | 1. 65% 2. 454 | 23.3 |
| 136 | 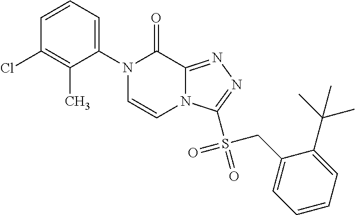 [SMILES: Cc1c(Cl)cccc1-n1ccn2c(S(=O)(=O)Cc3ccccc3C(C)(C)C)nnc2c1=O] | 3-(2-tert-butylbenzylsulfonyl)-7-(3- chloro-2-methylphenyl)- [1,2,4]triazolo[4,3-a]pyrazin-8(7H)- one | 1. 88% 2. 471 | 26.9 |
| 137 | 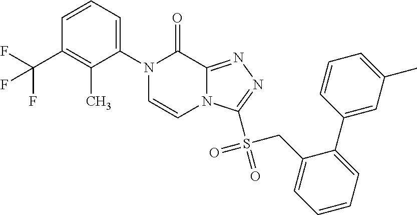 [SMILES: Cc1cccc(-c2ccccc2CS(=O)(=O)c2nnc3c(=O)n(-c4cccc(C(F)(F)F)c4C)ccn23)c1] | 7-(2-methyl-3- (trifluoromethyl)phenyl)-3-((3′- methylbiphenyl-2-yl)methylthio)- [1,2,4]triazolo[4,3-a]pyrazin-8(7H)- one | 1. 58% 2. 539 | 30.4 |
| 138 | 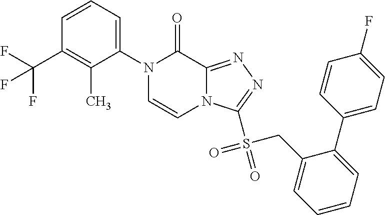 [SMILES: Cc1c(-n2ccn3c(S(=O)(=O)Cc4ccccc4-c4ccc(F)cc4)nnc3c2=O)cccc1C(F)(F)F] | 3 -((4′-fluorobiphenyl-2- yl)methylsulfonyl)-7-(2-methyl-3- (trifluoromethyl)phenyl)- [1,2,4]triazolo[4,3-a]pyrazin-8(7H)- one | 1. 85% 2. 543 | 30.8 |
| 139 | 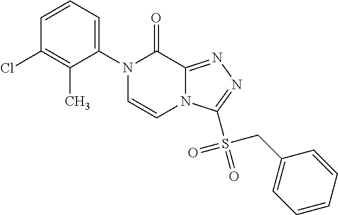 [SMILES: Cc1c(Cl)cccc1-n1ccn2c(S(=O)(=O)Cc3ccccc3)nnc2c1=O] | 3-(benzylsulfonyl)-7-(3-chloro-2- methylphenyl)-[1,2,4]triazolo[4,3- a]pyrazin-8(7H)-one | 1. 57% 2. 415 | 31.7 |
| 140 | 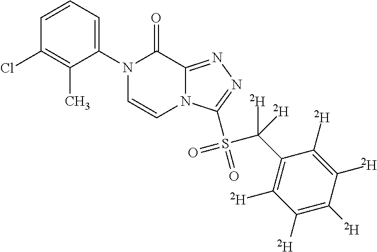 [SMILES: [2H]c1c([2H])c([2H])c(C([2H])([2H])S(=O)(=O)c2nnc3c(=O)n(-c4cccc(Cl)c4C)ccn23)c([2H])c1[2H]] | deuterated-7-(3-chloro-2- methylphenyl)-3-[(phenyl-(d5)methyl- (d2))sulfonyl]-1,2,4-triazolo[4,3- a]pyrazin-8(7H)-one | 1. 62% 2. 422 | 31.8 |
| 141 | 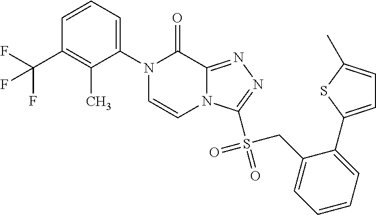 [SMILES: Cc1ccc(-c2ccccc2CS(=O)(=O)c2nnc3c(=O)n(-c4cccc(C(F)(F)F)c4C)ccn23)s1] | 7-(2-methyl-3- (trifluoromethyl)phenyl)-3-(2-(5- methylthiophen-2-yl)benzylthio)- [1,2,4]triazolo[4,3-a]pyrazin-8(7H)- one | 1. 64% 2. 545 | 37.0 |
| 142 | 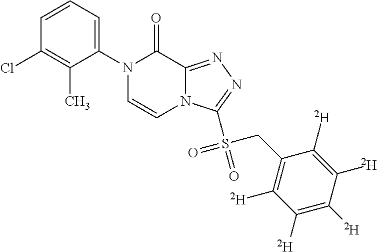 [SMILES: [2H]c1c([2H])c([2H])c(CS(=O)(=O)c2nnc3c(=O)n(-c4cccc(Cl)c4C)ccn23)c([2H])c1[2H]] | deuterated-7-(3-chloro-2- methylphenyl)-3-[(phenyl- (d5)methyl)sulfonyl]-1,2,4- triazolo[4,3-a]pyrazin-8(7H)-one | 1. 24% 2. 420 | 39.5 |
| 1443 | 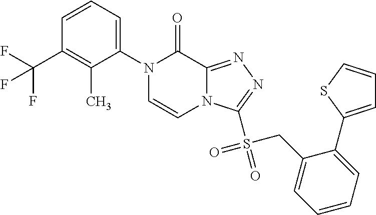 [SMILES: Cc1c(-n2ccn3c(S(=O)(=O)Cc4ccccc4-c4cccs4)nnc3c2=O)cccc1C(F)(F)F] | 7-(2-methyl-3- (trifluoromethyl)phenyl)-3-(2- (thiophen-2-yl)benzylsulfonyl)- [1,2,4]triazolo[4,3-a]pyrazin-8(7H)- one | 1. 52% 2. 530 | 46.2 |
| 144 | 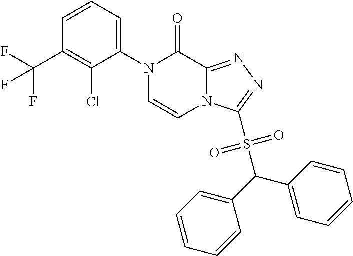 [SMILES: O=c1c2nnc(S(=O)(=O)C(c3ccccc3)c3ccccc3)n2ccn1-c1cccc(C(F)(F)F)c1Cl] | 3-(benzhydrylsulfonyl)-7-(2-chloro-3- (trifluoromethyl)phenyl)- [1,2,4]triazolo[4,3-a]pyrazin-8(7H)- one | 1. 56% 2. 545 | 130.1 |
| 145 | 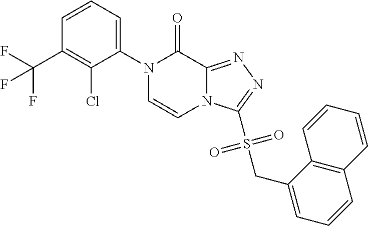 [SMILES: O=c1c2nnc(S(=O)(=O)Cc3cccc4ccccc34)n2ccn1-c1cccc(C(F)(F)F)c1Cl] | 7-(2-chloro-3- (trifluoromethyl)phenyl)-3- (naphthalen-1-ylmethylsulfonyl)- [1,2,4]triazolo[4,3-a]pyrazin-8(7H)- one | 1. 16% 2. 519 | 47.9 |
| 146 | 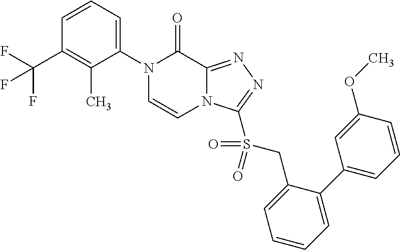 [SMILES: COc1cccc(-c2ccccc2CS(=O)(=O)c2nnc3c(=O)n(-c4cccc(C(F)(F)F)c4C)ccn23)c1] | 3-((3′-methoxybiphenyl-2- yl)methylsulfonyl)-7-(2-methyl-3- (trifluoromethyl)phenyl)- [1,2,4]triazolo[4,3-a]pyrazin-8(7H)- one | 1. 87% 2. 555 | 47.0 |
| 147 | 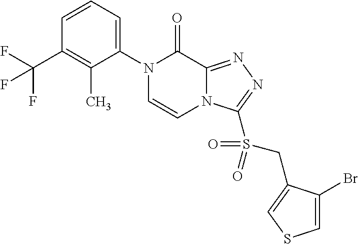 [SMILES: Cc1c(-n2ccn3c(S(=O)(=O)Cc4cscc4Br)nnc3c2=O)cccc1C(F)(F)F] | 3-((4-bromothiophen-3- yl)methylsulfonyl)-7-(2-methyl-3- (trifluoromethyl)phenyl)- [1,2,4]triazolo[4,3-a]pyrazin-8(7H)- one | 1. 76% 2. 535 | 45.8 |
| 148 | 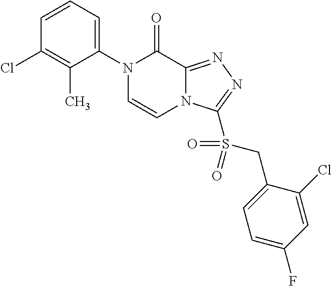 [SMILES: Cc1c(Cl)cccc1-n1ccn2c(S(=O)(=O)Cc3ccc(F)cc3Cl)nnc2c1=O] | 7-(3-chloro-2-methylphenyl)-3-(2- chloro-4-fluorobenzylsulfonyl)- [1,2,4]triazolo[4,3-a]pyrazin-8(7H)- one | 1. 24% 2. 467 | 47.0 |
| 149 | 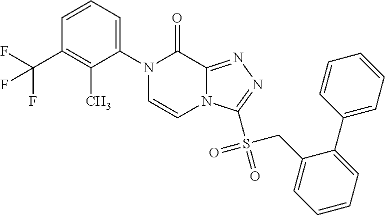 [SMILES: Cc1c(-n2ccn3c(S(=O)(=O)Cc4ccccc4-c4ccccc4)nnc3c2=O)cccc1C(F)(F)F] | 3-(biphenyl-2-ylmethylsulfonyl)-7-(2- methyl-3-(trifluoromethyl)phenyl)- [1,2,4]triazolo[4,3-a]pyrazin-8(7H)- one | 1. 49% 2. 525 | 48.0 |
| 150 | 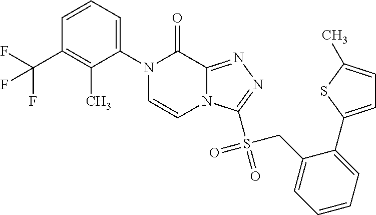 [SMILES: Cc1ccc(-c2ccccc2CS(=O)(=O)c2nnc3c(=O)n(-c4cccc(C(F)(F)F)c4C)ccn23)s1] | 7-(2-methyl-3- (trifluoromethyl)phenyl)-3-(2-(5- methylthiophen-2-yl)benzylsulfonyl)- [1,2,4]triazolo[4,3-a]pyrazin-8(7H)- one | 1. 27% 2. 545 | 48.9 |
| 151 | 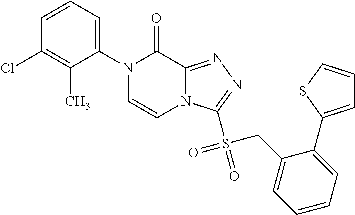 [SMILES: Cc1c(Cl)cccc1-n1ccn2c(S(=O)(=O)Cc3ccccc3-c3cccs3)nnc2c1=O] | 7-(3-chloro-2-methylphenyl)-3-(2- (thiophen-2-yl)benzylsulfonyl)- [1,2,4]triazolo[4,3-a]pyrazin-8(7H)- one | 1. 48% 2. 497 | 50.9 |
| 152 | 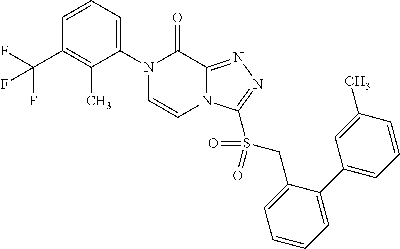 [SMILES: Cc1cccc(-c2ccccc2CS(=O)(=O)c2nnc3c(=O)n(-c4cccc(C(F)(F)F)c4C)ccn23)c1] | 7-(2-methyl-3- (trifluoromethyl)phenyl)-3-((3′- methylbiphenyl-2-yl)methylsulfonyl)- [1,2,4]triazolo[4,3-a]pyrazin-8(7H)- one | 1. 83% 2. 539 | 51.0 |
| 153 | 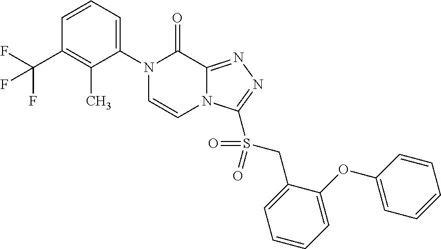 [SMILES: Cc1c(-n2ccn3c(S(=O)(=O)Cc4ccccc4Oc4ccccc4)nnc3c2=O)cccc1C(F)(F)F] | 7-(2-methyl-3- (trifluoromethyl)phenyl)-3-(2- phenoxybenzylsulfonyl)- [1,2,4]triazolo[4,3-a]pyrazin-8(7H)- one | 1. 76% 2. 541 | 51.5 |
| 154 | 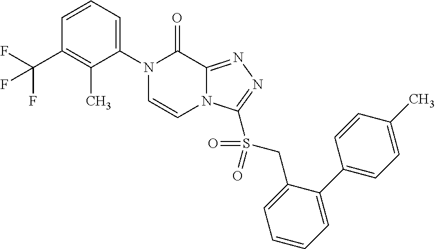 [SMILES: Cc1ccc(-c2ccccc2CS(=O)(=O)c2nnc3c(=O)n(-c4cccc(C(F)(F)F)c4C)ccn23)cc1] | 7-(2-methyl-3- (trifluoromethyl)phenyl)-3-((4′- methylbiphenyl-2-yl)methylsulfonyl)- [1,2,4]triazolo[4,3-a]pyrazin-8(7H)- one | 1. 79% 2. 539 | 51.9 |
| 155 | 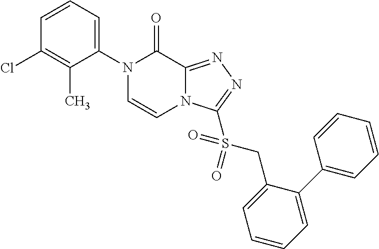 [SMILES: Cc1c(Cl)cccc1-n1ccn2c(S(=O)(=O)Cc3ccccc3-c3ccccc3)nnc2c1=O] | 3-(biphenyl-2-ylmethylsulfonyl)-7-(3- chloro-2-methylphenyl)- [1,2,4]triazolo[4,3-a]pyrazin-8(7H)- one | 1. 47% 2. 491 | 53.2 |
| 156 | 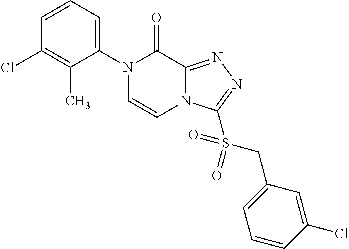 [SMILES: Cc1c(Cl)cccc1-n1ccn2c(S(=O)(=O)Cc3cccc(Cl)c3)nnc2c1=O] | 7-(3-chloro-2-methylphenyl)-3-(3- chlorobenzylsulfonyl)- [1,2,4]triazolo[4,3-a]pyrazin-8(7H)- one | 1. 53% 2. 449 | 119.3 |
| 157 | 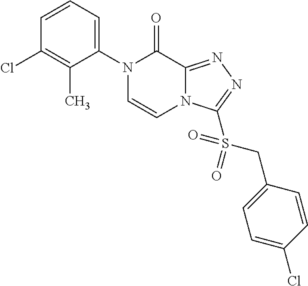 [SMILES: Cc1c(Cl)cccc1-n1ccn2c(S(=O)(=O)Cc3ccc(Cl)cc3)nnc2c1=O] | 7-(3-chloro-2-methylphenyl)-3-(4- chlorobenzylsulfonyl)- [1,2,4]triazolo[4,3-a]pyrazin-8(7H)- one | 1. 19% 2. 449 | 135.6 |
| 158 | 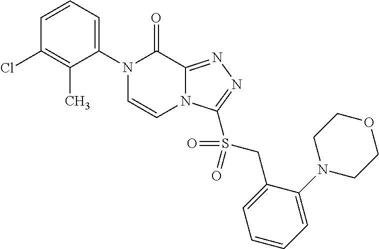 [SMILES: Cc1c(Cl)cccc1-n1ccn2c(S(=O)(=O)Cc3ccccc3N3CCOCC3)nnc2c1=O] | 7-(3-chloro-2-methylphenyl)-3-(2- morpholinobenzylsulfonyl)- [1,2,4]triazolo[4,3-a]pyrazin-8(7H)- one | 1. 29% 2. 500 | 189.5 |
| 159 | 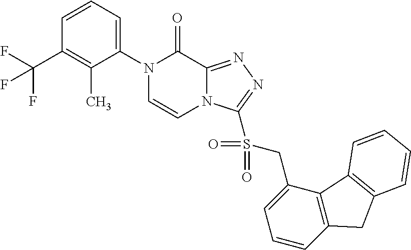 [SMILES: Cc1c(-n2ccn3c(S(=O)(=O)Cc4cccc5c4-c4ccccc4C5)nnc3c2=O)cccc1C(F)(F)F] | 3-((9H-fluoren-4-yl)methylsulfonyl)- 7-(2-methyl-3- (trifluoromethyl)phenyl)- [1,2,4]triazolo[4,3-a]pyrazin-8(7H)- one | 1. 55% 2. 537 | 1134 |
| 160 | 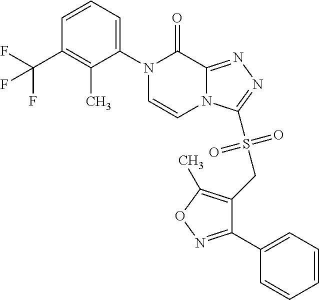 [SMILES: Cc1onc(-c2ccccc2)c1CS(=O)(=O)c1nnc2c(=O)n(-c3cccc(C(F)(F)F)c3C)ccn12] | 7-(2-methyl-3- (trifluoromethyl)phenyl)-3-((5- methyl-3-phenylisoxazol-4- yl)methylsulfonyl)-[1,2,4]triazolo[4,3- a]pyrazin-8(7H)-one | 1. 58% 2. 530 | 283.8 |
| 161 | 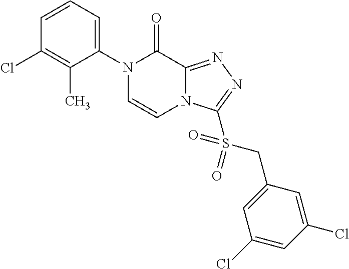 [SMILES: Cc1c(Cl)cccc1-n1ccn2c(S(=O)(=O)Cc3cc(Cl)cc(Cl)c3)nnc2c1=O] | 7-(3-chloro-2-methylphenyl)-3-(3,5- dichlorobenzylsulfonyl)- [1,2,4]triazolo[4,3-a]pyrazin-8(7H)- one | 1.11% 2. 483 | 335.2 |
| 162 | 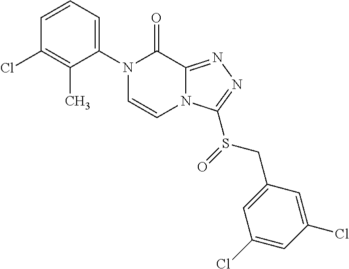 [SMILES: Cc1c(Cl)cccc1-n1ccn2c(S(=O)Cc3cc(Cl)cc(Cl)c3)nnc2c1=O] | 7-(3-chloro-2-methylphenyl)-3-(3,5- dichlorobenzylsulfinyl)- [1,2,4]triazolo[4,3-a]pyrazin-8(7H)- one | 1. 59% 2. 469 | 335.5 |
| 163 | 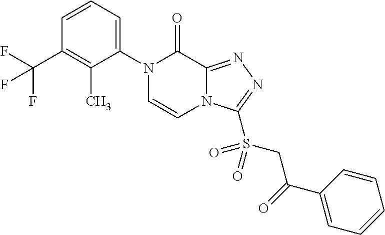 [SMILES: Cc1c(-n2ccn3c(S(=O)(=O)CC(=O)c4ccccc4)nnc3c2=O)cccc1C(F)(F)F] | 7-(2-methyl-3- (trifluoromethyl)phenyl)-3-(2-oxo-2- phenylethylsulfonyl)- [1,2,4]triazolo[4,3-a]pyrazin-8(7H)- one | 1. 12% 2. 477 | 3108 |
| 164 | 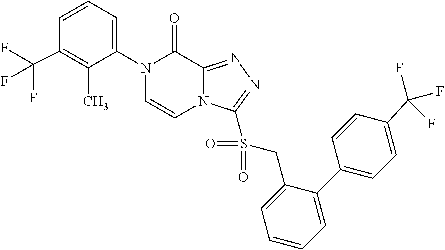 [SMILES: Cc1c(-n2ccn3c(S(=O)(=O)Cc4ccccc4-c4ccc(C(F)(F)F)cc4)nnc3c2=O)cccc1C(F)(F)F] | 7-(2-methyl-3- (trifluoromethyl)phenyl)-3-((4′- (trifluoromethyl)biphenyl-2- yl)methylsulfonyl)-[1,2,4]triazolo[4,3- a]pyrazin-8(7H)-one | 1. 85% 2. 593 | 628.7 |
| 165 | 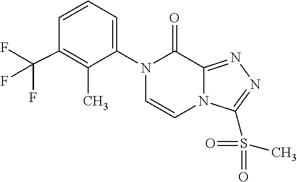 [SMILES: Cc1c(-n2ccn3c(S(C)(=O)=O)nnc3c2=O)cccc1C(F)(F)F] | 7-(2-methyl-3- (trifluoromethyl)phenyl)-3- (methylsulfonyl)-[1,2,4]triazolo[4,3- a]pyrazin-8(7H)-one | 1. 82% 2. 373 | 5685 |

### Example 7

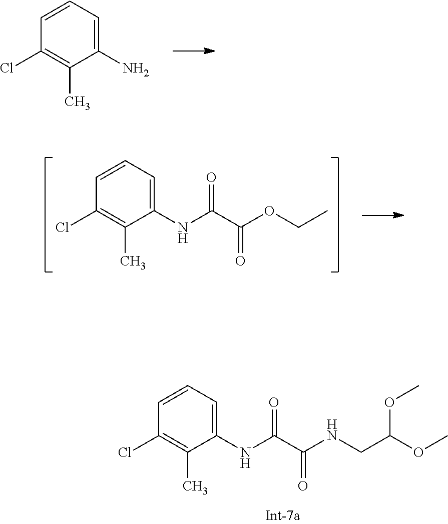

Into a 1-neck round-bottom flask was added 3-chloro-2-methylaniline (15.00 g, 105.9 mmol), ethyl oxalyl chloride (11.8 mL, 106 mmol) and triethylamine (14.8 mL, 106 mmol) in tetrahydrofuran (192 mL). The solution was stirred at room temperature for 1 hour. The mixture was filtered and the precipitate was washed with tetrahydrofuran (2×5 mL). The combined organic layer was concentrated to yield a brown oil which solidified on standing. The solid was redissolved in N,N-dimethylformamide (100 mL) and treated with aminoacetaldehyde dimethyl acetal (12.7 mL, 116 mmol). The solution was heated at 150° C. for 4 hours and cooled to room temperature and stirred for an additional 12 hours. The solution solidified during stirring at room temperature. The residue was diluted with isopropyl alcohol (170 mL) and stirred at room temperature for 1 hour. The precipitate was filtered, washed with isopropyl alcohol and dried to yield Int-7a (22.85 g, 72%) as a white solid which was used without further purification.

### Example 8

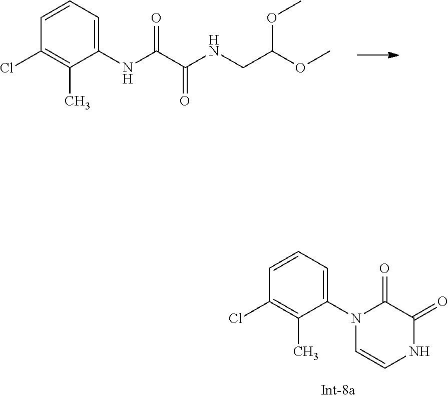 [SMILES: COC(CNC(=O)C(=O)Nc1cccc(Cl)c1C)OC ; Cc1c(Cl)cccc1-n1cc[nH]c(=O)c1=O]

Into a 1-neck round-bottom flask was added Int-7a (22.85 g, 75.98 mmol) in acetic acid (75.9 mL) and trifluoroacetic acid (7.59 mL, 98.5 mmol). The solution was heated at 115° C. for 6 hours. The solution was cooled to room temperature and isopropyl alcohol (189.7 mL) was added. Stirring was continued overnight at room temperature. The mixture was filtered and the precipitate was washed with isopropyl alcohol and dried under vacuum to yield Int-8a (15.01 g, 83%) as a light brown solid which was used without further purification.

### Example 9

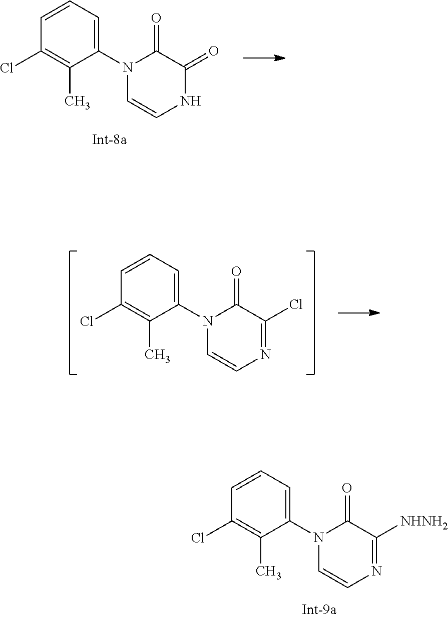

Into a 1-neck round-bottom flask was added Int-8a (15.0 g, 63 mmol) and N,N-diisopropylethylamine (21.1 mL, 121 mmol) in NN-dimethylformamide (62 mL). To the solution was added phosphoryl chloride (18.6 mL, 200 mmol) dropwise. The solution was heated at 95° C. for 1 hour. The solution was cooled to room temperature and poured over ice. The mixture was stirred at room temperature for 30 minutes. The mixture was filtered and the precipitate was washed with water. The precipitate was collected, dissolved in methylene chloride (250 mL) and dried (Na₂SO₄), filtered and concentrated under reduced pressure to yield 13.18 g of a brown solid.

The brown solid was dissolved in isopropyl alcohol (190 mL) and treated with hydrazine hydrate (30.8 mL, 634 mmol). The solution was heated at 85° C. for 1 hour. The solution was cooled to room temperature and diluted with water (263 mL). The mixture was stirred at room temperature for 30 minutes and filtered. The precipitate was dried to yield Int-9a (4.29 g) as a light brown solid. The aqueous layer was extracted with methylene chloride (3×25 mL). The combined organic extracts were dried (Na₂SO₄), filtered and concentrated under reduced pressure. The crude product was chromatographed on an Analogix purification system using a SF40 240 g column using 0-15% gradient elution of methanol in ethyl acetate to yield an additional batch of Int-9a (8.69 g) as a light brown solid for a combined overall yield of 12.98 g (82% overall for two steps).

### Example 10

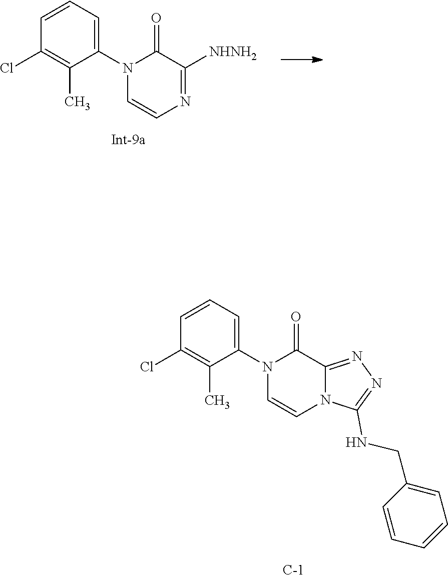

Into a 1-neck round-bottom flask was added Int-9a (0.214 g, 0.854 mmol) and (isothiocyanatomethyl)benzene (0.147 mL, 1.11 mmol) in ethanol (3.42 mL). The solution was heated at 75° C. for 1 hour. The mixture was cooled to room temperature. To the solution was added sodium acetate (0.105 g, 1.28 mmol) and methyl iodide (0.0691 mL, 1.11 mmol). The mixture was heated at 75° C. for 4 hours. The reaction was cooled to room temperature and stirred overnight. The formed precipitate was filtered and washed with ethanol (2×0.5 mL) and dried under vacuum to yield compound C-1 (0.23 g, 75%) as of a white solid. LC-MS: MW [MH⁺]=366.

By essentially the same procedure set forth in Example 10, the compounds given in Column 2 of Table 5, i.e., compounds 166-168, were prepared.

TABLE 5

| | | | 1. % Yield | |
|---|---|---|---|---|
| | | | 2. LC-MS: | P2X₇ Ca Flux |
| Example | Structural Formula | Chemical Name | MW [MH⁺] | hu IC₅₀ nM |
| 166 | 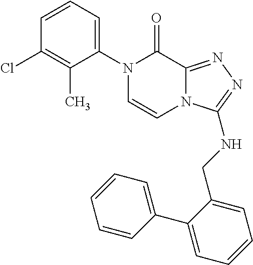 [SMILES: Cc1c(Cl)cccc1-n1ccn2c(NCc3ccccc3-c3ccccc3)nnc2c1=O] | 3-(biphenyl-2-ylmethylamino)-7-(3- chloro-2-methylphenyl)- [1,2,4]triazolo[4,3-a]pyrazin-8(7H)-one | 1. 65% 2. 442 | 585.1 |
| 167 | 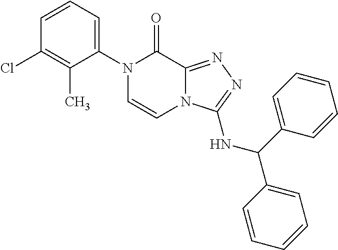 [SMILES: Cc1c(Cl)cccc1-n1ccn2c(NC(c3ccccc3)c3ccccc3)nnc2c1=O] | 3-(benzhydrylamino)-7-(3-chloro-2- methylphenyl)-[1,2,4]triazolo[4,3- a]pyrazin-8(7H)-one | 1. 47% 2. 442 | 1036 |
| 168 | 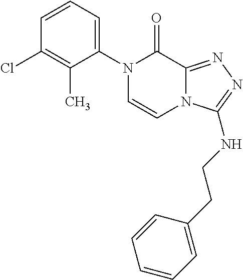 [SMILES: Cc1c(Cl)cccc1-n1ccn2c(NCCc3ccccc3)nnc2c1=O] | 7-(3-chloro-2-methylphenyl)-3- (phenethylamino)-[1,2,4]triazolo[4,3- a]pyrazin-8(7H)-one | 1. 78% 2. 380 | 3148 |

### Example 11

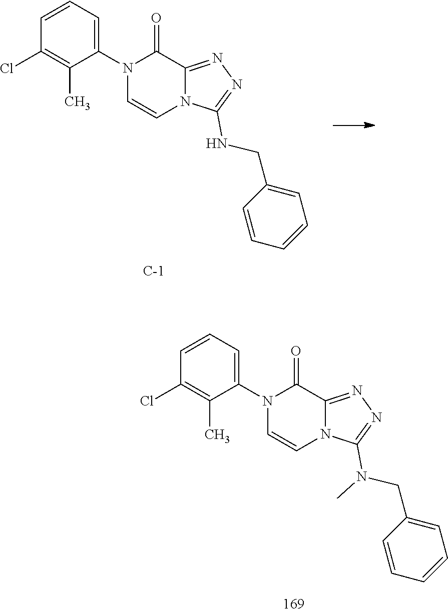

Into a 1-neck round-bottom flask was added compound C-1 (0.075 g, 0.20 mmol) in N,N-dimethylformamide (0.40 mL). The solution was cooled to 0° C. To the solution was added sodium hydride, 60% in mineral oil (0.0123 g). The solution was stirred at 0° C. for 30 minutes. Methyl iodide (0.017 mL, 0.266 mmol) was added and the solution was stirred at 0° C. for 2 hours. The mixture was diluted with water (3 mL) and stirred at room temperature for 10 minutes. The mixture was filtered and the precipitate was washed with water (2×1 mL), and dried under vacuum to yield compound 169 (0.067 g, 84%) as a cream-colored solid. LC-MS: MW [MH⁺]=380.

By essentially the same procedure set forth in Example 11 and appropriate alkylating agents, the compounds given in Column 2 of Table 6, i.e., compounds 170 and 171 were prepared.

TABLE 6

| | | | 1. % Yield | |
|---|---|---|---|---|
| | | | 2. LC-MS: | P2X₇ Ca Flux |
| Example | Structural Formula | Chemical Name | MW [MH⁺] | hu IC₅₀ nM |
| 169 | 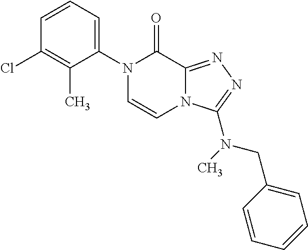 [SMILES: Cc1c(Cl)cccc1-n1ccn2c(N(C)Cc3ccccc3)nnc2c1=O] | 3-(benzyl(methyl)amino)-7-(3-chloro-2- methylphenyl)-[1,2,4]triazolo[4,3-a]pyrazin- 8(7H)-one | 1. 84% 2. 380 | 1588 |
| 170 | 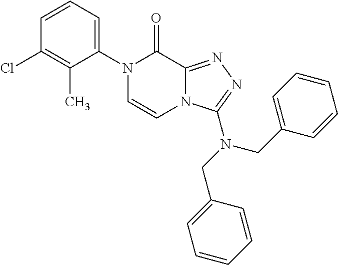 [SMILES: Cc1c(Cl)cccc1-n1ccn2c(N(Cc3ccccc3)Cc3ccccc3)nnc2c1=O] | 7-(3-chloro-2-methylphenyl)-3- (dibenzylamino)-[1,2,4]triazolo[4,3- a]pyrazin-8(7H)-one | 1. 30% 2. 456 | 106.4 |
| 171 | 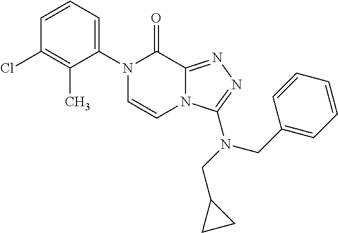 [SMILES: Cc1c(Cl)cccc1-n1ccn2c(N(Cc3ccccc3)CC3CC3)nnc2c1=O] | 3-(benzyl(cyclopropylmethyl)amino)-7-(3- chloro-2-methylphenyl)- [1,2,4]triazolo[4,3 - a]pyrazin-8(7H)-one | 1. 20% 2. 420 | 228.9 |

### Example 12

The ability of Triazolopyrazinone Compounds to inhibit P2X₇ function can be determined in Ca⁺⁺ flux assay using a P2X₇-HEK-293 cell line stably expressing the full length P2X₇ receptor. The assay protocol is as described in Cheewatrakoolpong et al., Biochem. Biophys. Res. Commun. 332:17-27 (2005) with minor modification, and is as summarized hereinafter. Briefly, cells were plated in poly-D-lysine-coated wells, and incubated at 37° C. for 24 hours. Cells were then incubated for 1 hour with a loading buffer which contains a dye sensitive to changes in Ca⁺⁺ concentration (lx Calcium Plus no wash dye from Molecular Devices, Sunnyvale, Calif.). The test compounds were added to the wells in the dye loading step. Plates containing the wells were transferred to a Fluorometric Imaging Plate Reader (Molecular Devices, Sunnyvale, Calif.). The cells were activated by the addition of the P2X₇ receptor agonist 2′(3) —O-(4-benzoylbenzoyl)adenosine 5′-triphosphate (Bz-ATP from Sigma-Aldrich, St. Louis, Mo.), and the fluorescence was monitored for 90 seconds. The fluorescent signal was determined as the total area over the 90 second run time. Using this protocol, representative Triazolopyrazinone Compounds, compounds 1471 were tested. Tables 3 to 6 show the concentration at which 50% of the maximum response in the Ca⁺⁺ was inhibited (IC₅₀) for each of the test compounds.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A compound having the Formula (I) 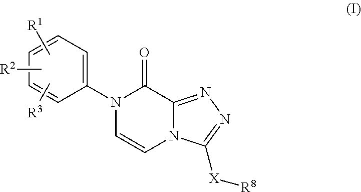
or a pharmaceutically acceptable salt thereof, wherein
X is —S(O)ₘ—, wherein m is 1 or 2;
R⁸ is —Y—R¹⁰;
Y is
(i) C₁-C₃ alkylene, wherein said alkylene is unsubstituted or substituted by one to two moieties independently selected from the group consisting of C₁-C₃ alkyl, and phenyl;
(ii) —CH₂C(O)—;
(iii) —N(H)C(O)—;
(iv) —C(O)N(H)—;
(v) —C(O)N(C₁-C₃ alkyl)-; or
(vi) Y is a bond such that R⁸ is bonded directly to X;
R¹⁰ is
(i) C₆-C₁₀ aryl;
(ii) fluorenyl;
(iii) 5- to 10-membered heteroaryl, wherein said heteroaryl contains from one to three heteroatoms selected from the group consisting of N, O, and S;
(iv) C₁-C₃ alkyl; or
(v) —O—(C₁-C₆)alkyl;
wherein said aryl, fluorenyl, or heteroaryl of R¹⁰ is unsubstituted or substituted with one or more moieties independently selected from the group consisting of halo, C₁-C₃ alkyl, C₁-C₃ alkoxy, C₁-C₃ fluoroalkoxy, C₁-C₃ fluoroalkyl, C₁-C₃ trifluoroalkylthio, cyano, carboxy, —C(O)—O—(C₁-C₃ alkyl), carbamyl,
—C(O)—(C₁-C₃)alkyl,
—C(O)N(H)(C₁-C₃ alkyl), —C(O)N(C₁-C₃ alkyl)₂, —NHC(O)—(C₁-C₃ alkyl), and -MR¹¹, wherein
M is C₁-C₃ alkylene, —(C₁-C₃ alkylene)-O—, —O—, —CH₂S(O)₂—, —CH₂S(O)—, or a bond;
R¹¹ is
(i) phenyl;
(ii) 5- to 6-membered heterocyclyl having one to two heteroatoms independently selected from the group consisting of N and O; and
(iii) 5- to 6-membered heteroaryl having one to three heteroatoms independently selected from the group consisting of N, O, and S;
wherein said phenyl, heterocyclyl, or heteroaryl of R¹¹ is unsubstituted or substituted by one to three moieties independently selected from the group consisting of halo, C₁-C₃ alkyl, trifluoromethyl, trifluoromethoxy, C₁-C₃ alkoxy, cyano, carbamyl, amino, C₁-C₃ alkylamino, C₁-C₃ dialkylamino, acetamido, C₁-C₆ alkoxycarbonyl, and —B(OH)₂;
R¹, R², and R³ are independently selected from the group consisting of hydrogen, halo, C₁-C₃ alkyl, C₁-C₃ alkoxy, trifluoromethyl, trifluoromethoxy, cyano, carbamyl, amino, C₁-C₃ alkylamino, C₁-C₃ dialkylamino, and acetamido.

2. The compound of claim 1 or a pharmaceutically acceptable salt thereof, wherein X is —S(O)₂—.

3. The compound of claim 1 or a pharmaceutically acceptable salt thereof, wherein Y is
(i) C₁-C₃ alkylene, wherein said alkylene is unsubstituted or substituted by one to two moieties independently selected from the group consisting of C₁-C₃ alkyl and phenyl;
(ii) —CH₂C(O)—; or
(iii) a bond.

4. The compound of claim 3 or a pharmaceutically acceptable salt thereof, wherein Y is methylene, wherein said alkylene is unsubstituted or substituted by one phenyl.

5. The compound of claim 1 or a pharmaceutically acceptable salt thereof, wherein R¹⁰ is
(i) C₆-C₁₀ aryl;
(ii) fluorenyl; or
(iii) 5- to 10-membered heteroaryl, wherein said heteroaryl contains from one to three heteroatoms independently selected from the group consisting of N, O, and S;
wherein said aryl, fluorenyl, or heteroaryl of R¹⁰ is unsubstituted or substituted with one to three moieties independently selected from the group consisting of halo, C₁-C₄ alkyl, C₁-C₃ alkoxy, C₁-C₃ fluoroalkoxy, C₁-C₃ fluoroalkyl, C₁-C₃ trifluoroalkylthio, cyano, carboxy, —C(O)—O—(C₁-C₃ alkyl), carbamyl,
—C(O)—(C₁-C₃)alkyl, —C(O)N(H)(C₁-C₃ alkyl), —C(O)N(C₁-C₃ alkyl)₂, —NHC(O)—(C₁-C₃ alkyl), and -MR¹¹.

6. The compound of claim 1 or a pharmaceutically acceptable salt thereof, wherein R¹⁰ is phenyl, napthyl, fluorenyl, thienyl or isoxazolyl;
wherein said phenyl, napthyl, thienyl, fluorenyl, or isoxazolyl of R¹⁰ is unsubstituted or substituted with one to two moieties independently selected from the group consisting of halo, C₁-C₄ alkyl, C₁-C₃ alkoxy, C₁-C₃ fluoroalkyl, morpholinyl, phenyl, chlorophenyl, fluorophenyl, (C₁-C₄ alkyl)phenyl, C₁-C₃ alkoxy)phenyl, trifluoromethylphenyl, phenoxyphenyl, napthyl, thienyl, bromothienyl, and (C₁-C₃ alkyl)thienyl.

7. The compound of claim 1 or a pharmaceutically acceptable salt thereof, wherein R¹, R², and R³ are independently selected from the group consisting of hydrogen, halo, C₁-C₃ alkyl, and trifluoromethyl.

8. The compound of claim 1 having the formula (IA) 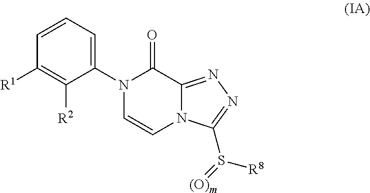
or a pharmaceutically acceptable salt thereof, wherein
m is 1 or 2;
R⁸ is —Y—R¹⁰;
Y is methylene, wherein said methylene is unsubstituted or substituted by one to two moieties independently selected from the group consisting of C₁-C₃ alkyl and phenyl:
R¹⁰ is phenyl, napthyl, fluorenyl, thienyl or isoxazolyl;
wherein said phenyl, napthyl, thienyl, or isoxazolyl of R¹⁰ is unsubstituted or substituted with one to two moieties independently selected from the group consisting of halo, C₁-C₄ alkyl, C₁-C₃ alkoxy, C₁-C₃ fluoroalkyl, morpholinyl, phenyl, chlorophenyl, fluorophenyl, (C₁-C₄ alkyl)phenyl, (C₁-C₃ alkoxy)phenyl, trifluoromethylphenyl, phenoxyphenyl, napthyl, thienyl, bromothienyl, and (C₁-C₃ alkyl)thienyl;
R¹ and R² are independently selected from the group consisting of hydrogen, halo, C₁-C₃ alkyl, and trifluoromethyl.

9. The compound of claim 8 or a pharmaceutically acceptable salt thereof, wherein m is 2.

10. The compound of claim 8, wherein R¹ is trifluoromethyl or chloro, and R² is methyl or chloro.

11. The compound of claim 1 or a pharmaceutically acceptable salt thereof, wherein the compound is selected from the group consisting of:
116 3-(benzylsulfonyl)-7-(2-methyl-3-(trifluoromethyl)phenyl)-[1,2,4]triazolo[4,3-a]pyrazin-8(7H)-one
117 3-(benzylsulfinyl)-7-(2-methyl-3-(trifluoromethyl)phenyl)-[1,2,4]triazolo[4,3-a]pyrazin-8(7H)-one;
118 3-((4′-chlorobiphenyl-2-yl)methylsulfonyl)-7-(2-methyl-3-(trifluoromethyl)phenyl)-[1,2,4]triazolo[4,3-a]pyrazin-8(7H)-one;
119 7-(2-chloro-3-(trifluoromethyl)phenyl)-3-(2-chlorobenzylsulfonyl)-[1,2,4]triazolo[4,3-a]pyrazin-8(7H)-one;
120 3-(2-fluorobenzylsulfonyl)-7-(2-methyl-3-(trifluoromethyl)phenyl)-[1,2,4]triazolo[4,3-a]pyrazin-8(7H)-one;
121 3-(2-chlorobenzylsulfonyl)-7-(2-methyl-3-(trifluoromethyl)phenyl)-[1,2,4]triazolo[4,3-a]pyrazin-8(7H)-one;
122 7-(2-methyl-3-(trifluoromethyl)phenyl)-3-(thiophen-2-ylmethylsulfonyl)-[1,2,4]triazolo[4,3-a]pyrazin-8(7H)-one;
123 7-(2-methyl-3-(trifluoromethyl)phenyl)-3-(thiophen-3-ylmethylsulfonyl)-[1,2,4]triazolo[4,3-a]pyrazin-8(7H)-one;
124 3-(2-chloro-3-fluorobenzylsulfonyl)-7-(2-methyl-3-(trifluoromethyl)phenyl)-[1,2,4]triazolo[4,3-a]pyrazin-8(7H)-one;
125 3-((2-bromothiophen-3-yl)methylsulfonyl)-7-(2-methyl-3-(trifluoromethyl)phenyl)-[1,2,4]triazolo[4,3-a]pyrazin-8(7H)-one;
126 3-(biphenyl-2-ylmethylthio)-7-(2-methyl-3-(trifluoromethyl)phenyl)-[1,2,4]triazolo[4,3-a]pyrazin-8(7H)-one;
127 3-(benzylsulfonyl)-7-(2-chloro-3-(trifluoromethyl)phenyl)-[1,2,4]triazolo[4,3-a]pyrazin-8(7H)-one;
128 3-(2-bromobenzylsulfonyl)-7-(3-chloro-2-methylphenyl)-[1,2,4]triazolo[4,3-a]pyrazin-8(7H)-one;
129 3-(2-tert-butylbenzylsulfonyl)-7-(2-methyl-3-(trifluoromethyl)phenyl)-[1,2,4]triazolo[4,3-a]pyrazin-8(7H)-one;
130 7-(3-chloro-2-methylphenyl)-3-(2-chloro-3-fluorobenzylsulfonyl)-[1,2,4]triazolo[4,3-a]pyrazin-8(7H)-one;
131 3-(2-bromobenzylsulfonyl)-7-(2-methyl-3-(trifluoromethyl)phenyl)-[1,2,4]triazolo[4,3-a]pyrazin-8(7H)-one;
132 3-((5-chlorothiophen-2-yl)methylsulfonyl)-7-(2-methyl-3-(trifluoromethyl)phenyl)-[1,2,4]triazolo[4,3-a]pyrazin-8(7H)-one;
133 3-(2-iodobenzylsulfonyl)-7-(2-methyl-3-(trifluoromethyl)phenyl)-[1,2,4]triazolo[4,3-a]pyrazin-8(7H)-one;
134 7-(3-chloro-2-methylphenyl)-3-(2-chlorobenzylsulfonyl)-[1,2,4]triazolo[4,3-a]pyrazin-8(7H)-one;
135 deuterated-7-[2-methyl-3-(trifluoromethyl)phenyl]-3-[(phenyl-(d5)-methyl)sulfonyl]-1,2,4-triazolo[4,3-a]pyrazin-8(7H)-one;
136 3-(2-tert-butylbenzylsulfonyl)-7-(3-chloro-2-methylphenyl)-[1,2,4]triazolo[4,3-a]pyrazin-8(7H)-one;
137 7-(2-methyl-3-(trifluoromethyl)phenyl)-3-((3′-methylbiphenyl-2-yl)methylthio)-[1,2,4]triazolo[4,3-a]pyrazin-8(7H)-one;
138 3-((4′-fluorobiphenyl-2-yl)methylsulfonyl)-7-(2-methyl-3-(trifluoromethyl)phenyl)-[1,2,4]triazolo[4,3-a]pyrazin-8(7H)-one;
139 3-(benzylsulfonyl)-7-(3-chloro-2-methylphenyl)-[1,2,4]triazolo[4,3-a]pyrazin-8(7H)-one;
140 deuterated-7-(3-chloro-2-methylphenyl)-3-[(phenyl-(d5)methyl-(d2))-sulfonyl]-1,2,4-triazolo[4,3-a]pyrazin-8(7H)-one;
141 7-(2-methyl-3-(trifluoromethyl)phenyl)-3-(2-(5-methylthiophen-2-yl)benzylthio)-[1,2,4]triazolo[4,3-a]pyrazin-8(7H)-one;
142 deuterated-7-(3-chloro-2-methylphenyl)-3-[(phenyl-(d5)methyl)sulfonyl]-1,2,4-triazolo[4,3-a]pyrazin-8(7H)-one;
143 7-(2-methyl-3-(trifluoromethyl)phenyl)-3-(2-(thiophen-2-yl)benzylsulfonyl)-[1,2,4]triazolo[4,3-a]pyrazin-8(7H)-one;
144 3-(benzhydrylsulfonyl)-7-(2-chloro-3-(trifluoromethyl)phenyl)-[1,2,4]triazolo[4,3-a]pyrazin-8(7H)-one;
145 7-(2-chloro-3-(trifluoromethyl)phenyl)-3-(naphthalen-1-ylmethylsulfonyl)-[1,2,4]triazolo[4,3-a]pyrazin-8(7H)-one;
146 3-((3′-methoxybiphenyl-2-yl)methylsulfonyl)-7-(2-methyl-3-(trifluoromethyl)phenyl)-[1,2,4]triazolo[4,3-a]pyrazin-8(7H)-one;
147 3-((4-bromothiophen-3-yl)methylsulfonyl)-7-(2-methyl-3-(trifluoromethyl)phenyl)-[1,2,4]triazolo[4,3-a]pyrazin-8(7H)-one;
148 7-(3-chloro-2-methylphenyl)-3-(2-chloro-4-fluorobenzylsulfonyl)-[1,2,4]triazolo[4,3-a]pyrazin-8(7H)-one;
149 3-(biphenyl-2-ylmethylsulfonyl)-7-(2-methyl-3-(trifluoromethyl)phenyl)-[1,2,4]triazolo[4,3-a]pyrazin-8(7H)-one;
150 7-(2-methyl-3-(trifluoromethyl)phenyl)-3-(2-(5-methylthiophen-2-yl)benzylsulfonyl)-[1,2,4]triazolo[4,3-a]pyrazin-8(7H)-one;
151 7-(3-chloro-2-methylphenyl)-3-(2-(thiophen-2-yl)benzylsulfonyl)-[1,2,4]triazolo[4,3-a]pyrazin-8(7H)-one;
152 7-(2-methyl-3-(trifluoromethyl)phenyl)-3-((3′-methylbiphenyl-2-yl)methylsulfonyl)-[1,2,4]triazolo[4,3-a]pyrazin-8(7H)-one;
153 7-(2-methyl-3-(trifluoromethyl)phenyl)-3-(2-phenoxybenzylsulfonyl)-[1,2,4]triazolo[4,3-a]pyrazin-8(7H)-one;
154 7-(2-methyl-3-(trifluoromethyl)phenyl)-3-((4′-methylbiphenyl-2-yl)methylsulfonyl)-[1,2,4]triazolo[4,3-a]pyrazin-8(7H)-one;
155 3-(biphenyl-2-ylmethylsulfonyl)-7-(3-chloro-2-methylphenyl)-[1,2,4]triazolo[4,3-a]pyrazin-8(7H)-one;
156 7-(3-chloro-2-methylphenyl)-3-(3-chlorobenzylsulfonyl)-[1,2,4]triazolo[4,3-a]pyrazin-8(7H)-one;
157 7-(3-chloro-2-methylphenyl)-3-(4-chlorobenzylsulfonyl)-[1,2,4]triazolo[4,3-a]pyrazin-8(7H)-one;
158 7-(3-chloro-2-methylphenyl)-3-(2-morpholinobenzylsulfonyl)-[1,2,4]triazolo[4,3-a]pyrazin-8(7H)-one;
159 3-((9H-fluoren-4-yl)methylsulfonyl)-7-(2-methyl-3-(trifluoromethyl)phenyl)-[1,2,4]triazolo[4,3-a]pyrazin-8(7H)-one;
160 7-(2-methyl-3-(trifluoromethyl)phenyl)-3-((5-methyl-3-phenylisoxazol-4-yl)methylsulfonyl)-[1,2,4]triazolo[4,3-a]pyrazin-8(7H)-one;
161 7-(3-chloro-2-methylphenyl)-3-(3,5-dichlorobenzylsulfonyl)-[1,2,4]triazolo[4,3-a]pyrazin-8(7H)-one;
162 7-(3-chloro-2-methylphenyl)-3-(3,5-dichlorobenzylsulfinyl)-[1,2,4]triazolo[4,3-a]pyrazin-8(7H)-one;
163 7-(2-methyl-3-(trifluoromethyl)phenyl)-3-(2-oxo-2-phenylethylsulfonyl)-[1,2,4]triazolo[4,3-a]pyrazin-8(7H)-one;
164 7-(2-methyl-3-(trifluoromethyl)phenyl)-3-((4′-(trifluoromethyl)biphenyl-2-yl)methylsulfonyl)-[1,2,4]triazolo[4,3-a]pyrazin-8(7H)-one; and
165 7-(2-methyl-3-(trifluoromethyl)phenyl)-3-(methylsulfonyl)-[1,2,4]triazolo[4,3-a]pyrazin-8(7H)-one.

12. A compound having the Formula (I) 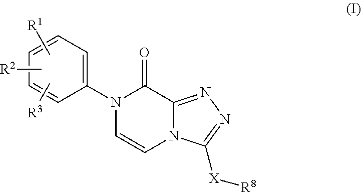
or a pharmaceutically acceptable salt thereof, wherein
X is
(i) —S(O)ₘ—, wherein m is 0, 1, or 2; or
(ii) —N(R⁹)—;
R⁸ is —Y—R¹⁰;
Y is
(i) C₁-C₃ alkylene, wherein said alkylene is unsubstituted or substituted by one to two moieties independently selected from the group consisting of C₁-C₃ alkyl and phenyl;
(ii) —CH₂C(O)—;
(iii) —N(H)C(O)—;
(iv) —C(O)N(H)—;
(v) —C(O)N(C₁-C₃ alkyl)-; or
(vi) Y is a bond such that R⁸ is bonded directly to X;
R¹⁰ is
(i) C₆-C₁₀ aryl;
(ii) fluorenyl;
(iii) 5- to 10-membered heteroaryl, wherein said heteroaryl contains from one to three heteroatoms independently selected from the group consisting of N, O, and S;
(iv) C₁-C₃ alkyl; or
(v) —O—(C₁-C₆)alkyl;
wherein said aryl, fluorenyl, or heteroaryl of R¹⁰ is unsubstituted or substituted with one or more moieties independently selected from the group consisting of halo, C₁-C₃ alkyl, C₁-C₃ alkoxy, C₁-C₃ fluoroalkoxy,
C₁-C₃ fluoroalkyl, C₁-C₃ trifluoroalkylthio, cyano, carboxy,
—C(O)—O—(C₁-C₃ alkyl), carbamyl, —C(O)—(C₁-C₃)alkyl,
—C(O)N(H)(C₁-C₃ alkyl), —C(O)N(C₁-C₃ alkyl)₂, —NHC(O)—(C₁-C₃ alkyl), or -MR¹¹,
wherein
M is C₁-C₃ alkylene, —(C₁-C₃ alkylene)-O—, —O—, —CH₂S(O)₂—, —CH₂S(O)—, or a bond;
R¹¹ is
(i) phenyl;
(ii) 5- to 6-membered heterocyclyl having one to two heteroatoms independently selected from the group consisting of N and O; and
(iii) 5- to 6-membered heteroaryl having one to three heteroatoms independently selected from the group consisting of N, O, and S;
wherein said phenyl, heterocyclyl, or heteroaryl of R¹¹ is unsubstituted or substituted by one to three moieties independently selected from the group consisting of halo, C₁-C₃ alkyl, trifluoromethyl, trifluoromethoxy, C₁-C₃ alkoxy, cyano, carbamyl, amino, C₁-C₃ alkylamino, C₁-C₃ dialkylamino, acetamido, C₁-C₆ alkoxycarbonyl, and —B(OH)₂;
R⁹ is H, C₁-C₃ alkyl, —(C₁-C₃ alkylene)-(C₃-C₇ cycloalkyl), or —(C₁-C₃ alkylene)-phenyl; and
R¹, R², and R³ are independently selected from the group consisting of hydrogen, halo, C₁-C₃ alkyl, C₁-C₃ alkoxy, trifluoromethyl, trifluoromethoxy, cyano, carbamyl, amino, C₁-C₃ alkylamino, C₁-C₃ dialkylamino, and acetamido; and
wherein the compound is selected from the group consisting of:
1 3-(benzylthio)-7-(2-methyl-3-(trifluoromethyl)phenyl)-[1,2,4]triazolo[4,3-a]pyrazin-8(7H)-one;
2 7-(3-chloro-2-methylphenyl)-3-(3,4-difluorobenzylthio)-[1,2,4]triazolo[4,3-a]pyrazin-8(7H)-one;
3 3-((1-methyl-1H-benzo[d]imidazol-2-yl)methylthio)-7-(2-methyl-3-(trifluoromethyl)phenyl)-[1,2,4]triazolo[4,3-a]pyrazin-8(7H)-one;
4 3-((9H-fluoren-4-yl)methylthio)-7-(2-methyl-3-(trifluoromethyl)phenyl)-[1,2,4]triazolo[4,3-a]pyrazin-8(7H)-one;
7-(2-methyl-3-(trifluoromethyl)phenyl)-3-((3′-methylbiphenyl-2-yl)methylthio)-[1,2,4]triazolo[4,3-a]pyrazin-8(7H)-one;
6 7-(2-methyl-3-(trifluoromethyl)phenyl)-3-(2-(5-methylthiophen-2-yl)benzylthio)-[1,2,4]triazolo[4,3-a]pyrazin-8(7H)-one;
7 7-(2-methyl-3-(trifluoromethyl)phenyl)-3-((4′-methylbiphenyl-2-yl)methylthio)-[1,2,4]triazolo[4,3-a]pyrazin-8(7H)-one;
8 7-(2-methyl-3-(trifluoromethyl)phenyl)-3-((4′-(trifluoromethyl)biphenyl-2-yl)methylthio)-[1,2,4]triazolo[4,3-a]pyrazin-8(7H)-one;
9 3-((4′-chlorobiphenyl-2-yl)methylthio)-7-(2-methyl-3-(trifluoromethyl)phenyl)-[1,2,4]triazolo[4,3-a]pyrazin-8(7H)-one;
10 3-((4′-fluorobiphenyl-2-yl)methylthio)-7-(2-methyl-3-(trifluoromethyl)phenyl)-[1,2,4]triazolo[4,3-a]pyrazin-8(7H)-one;
11 3-(2-iodobenzylthio)-7-(2-methyl-3-(trifluoromethyl)phenyl)-[1,2,4]triazolo[4,3-a]pyrazin-8(7H)-one;
12 3-(2-bromobenzylthio)-7-(2-methyl-3-(trifluoromethyl)phenyl)-[1,2,4]triazolo[4,3-a]pyrazin-8(7H)-one;
13 3-(2-chloro-3-fluorobenzylthio)-7-(2-methyl-3-(trifluoromethyl)phenyl)-[1,2,4]triazolo[4,3-a]pyrazin-8(7H)-one;
14 7-(2-methyl-3-(trifluoromethyl)phenyl)-3-(thiophen-3-ylmethylthio)-[1,2,4]triazolo[4,3-a]pyrazin-8(7H)-one;
15 7-(2-methyl-3-(trifluoromethyl)phenyl)-3-(thiophen-2-ylmethylthio)-[1,2,4]triazolo[4,3-a]pyrazin-8(7H)-one;
16 7-(3-chloro-2-methylphenyl)-3-(2-chloro-3-fluorobenzylthio)-[1,2,4]triazolo[4,3-a]pyrazin-8(7H)-one;
17 7-(2-methyl-3-(trifluoromethyl)phenyl)-3-((5-methyl-3-phenylisoxazol-4-yl)methylthio)-[1,2,4]triazolo[4,3-a]pyrazin-8(7H)-one;
18 3-(2-tert-butylbenzylthio)-7-(2-methyl-3-(trifluoromethyl)phenyl)-[1,2,4]triazolo[4,3-a]pyrazin-8(7H)-one;
19 3-(2-tert-butylbenzylthio)-7-(3-chloro-2-methylphenyl)-[1,2,4]triazolo[4,3-a]pyrazin-8(7H)-one;
20 7-(2-methyl-3-(trifluoromethyl)phenyl)-3-(2-phenoxybenzylthio)-[1,2,4]triazolo[4,3-a]pyrazin-8(7H)-one;
21 deuterated-7-[2-methyl-3-(trifluoromethyl)phenyl]-3-[phenyl-(d5)-methyl)thio]-1,2,4-triazolo[4,3-a]pyrazin-8(7H)-one;
22 7-(2-methyl-3-(trifluoromethyl)phenyl)-3-(2-oxo-1,2-diphenylethylthio)-[1,2,4]triazolo[4,3-a]pyrazin-8(7H)-one;
23 7-(2-methyl-3-(trifluoromethyl)phenyl)-3-(2-(thiophen-2-yl)benzylthio)-[1,2,4]triazolo[4,3-a]pyrazin-8(7H)-one;
24 2-((7-(3-chloro-2-methylphenyl)-8-oxo-7,8-dihydro-[1,2,4]triazolo[4,3-a]pyrazin-3-ylthio)methyl)phenylboronic acid;
25 3-(benzhydrylthio)-7-(2-chloro-3-(trifluoromethyl)phenyl)-[1,2,4]triazolo[4,3-a]pyrazin-8(7H)-one;
26 3-(9H-fluoren-9-ylthio)-7-(2-chloro-3-(trifluoromethyl)phenyl)-[1,2,4]triazolo[4,3-a]pyrazin-8(7H)-one;
27 7-(2-chloro-3-(trifluoromethyl)phenyl)-3-(naphthalen-1-ylmethylthio)-[1,2,4]triazolo[4,3-a]pyrazin-8(7H)-one;
28 3-(2-chlorobenzylthio)-7-(2-methyl-3-(trifluoromethyl)phenyl)-[1,2,4]triazolo[4,3-a]pyrazin-8(7H)-one;
29 3-(benzylthio)-7-(naphthalen-1-yl)-[1,2,4]triazolo[4,3-a]pyrazin-8(7H)-one;
30 7-(3-chloro-2-methylphenyl)-3-(2-phenoxybenzylthio)-[1,2,4]triazolo[4,3-a]pyrazin-8(7H)-one;
31 tert-butyl 4-(247-(3-chloro-2-methylphenyl)-8-oxo-7,8-dihydro-[1,2,4]triazolo[4,3-a]pyrazin-3-ylthio)methyl)phenyl)piperazine-1-carboxylate;
32 7-(3-chloro-2-methylphenyl)-3-(2-morpholinobenzylthio)-[1,2,4]triazolo[4,3-a]pyrazin-8(7H)-one;
33 deuterated-7-(3-chloro-2-methylphenyl)-3-[(phenyl-(d5)-methyl)thio]-1,2,4-triazolo[4,3-a]pyrazin-8(7H)-one;
34 deuterated-7-(3-chloro-2-methylphenyl)-3-[(phenyl-(d5)-methyl-(d2))thio]-1,2,4-triazolo[4,3-a]pyrazin-8(7H)-one;
35 7-(3-chloro-2-methylphenyl)-3-(2-ethylbenzylthio)-[1,2,4]triazolo[4,3-a]pyrazin-8(7H)-one;
36 7-(3-chloro-2-methylphenyl)-3-(2-(pyridin-3-yl)benzylthio)-[1,2,4]triazolo[4,3-a]pyrazin-8(7H)-one;
37 7-(3-chloro-2-methylphenyl)-3-(2-(furan-2-yl)benzylthio)-[1,2,4]triazolo[4,3-a]pyrazin-8(7H)-one;
38 3-(2-((1H-1,2,4-triazol-1-yl)methyl)benzylthio)-7-(3-chloro-2-methylphenyl)-[1,2,4]triazolo[4,3-a]pyrazin-8(7H)-one;
39 7-(3-chloro-2-methylphenyl)-3-(2-(morpholinomethyl)benzylthio)-[1,2,4]triazolo[4,3-a]pyrazin-8(7H)-one;
40 7-(3-chloro-2-methylphenyl)-3-(2-(thiophen-2-yl)benzylthio)-[1,2,4]triazolo[4,3-a]pyrazin-8(7H)-one;
41 7-(3-chloro-2-methylphenyl)-3-(2-isopropylbenzylthio)-[1,2,4]triazolo[4,3-a]pyrazin-8(7H)-one;
42 7-(3-chloro-2-methylphenyl)-3-(2-(phenoxymethyl)benzylthio)-[1,2,4]triazolo[4,3-a]pyrazin-8(7H)-one;
43 7-(3-chloro-2-methylphenyl)-3-(2-(phenylsulfonylmethyl)benzylthio)-[1,2,4]triazolo[4,3-a]pyrazin-8(7H)-one;
44 7-(3-chloro-2-methylphenyl)-3-(2-phenethylbenzylthio)-[1,2,4]triazolo[4,3-a]pyrazin-8(7H)-one;
45 3-(2-benzylbenzylthio)-7-(3-chloro-2-methylphenyl)-[1,2,4]triazolo[4,3-a]pyrazin-8(7H)-one;
46 3-(biphenyl-3-ylmethylthio)-7-(3-chloro-2-methylphenyl)-[1,2,4]triazolo[4,3-a]pyrazin-8(7H)-one;
47 7-(2-chloro-3-(trifluoromethyl)phenyl)-3-(2-methylbenzylthio)-[1,2,4]triazolo[4,3-a]pyrazin-8(7H)-one;
48 3-(biphenyl-2-ylmethylthio)-7-(2-chloro-3-(trifluoromethyl)phenyl)-[1,2,4]triazolo[4,3-a]pyrazin-8(7H)-one;
49 3-(biphenyl-2-ylmethylthio)-7-(2-methyl-3-(trifluoromethyl)phenyl)-[1,2,4]triazolo[4,3-a]pyrazin-8(7H)-one;
50 S-7-(3-chloro-2-methylphenyl)-8-oxo-7,8-dihydro-[1,2,4]triazolo[4,3-a]pyrazin-3-yl methyl(phenyl)carbamothioate;
51 7-(2-methyl-3-(trifluoromethyl)phenyl)-3-(2-(trifluoromethoxy)benzylthio)-[1,2,4]triazolo[4,3-a]pyrazin-8(7H)-one;
52 7-(3-chloro-2-methylphenyl)-3-(2-(trifluoromethylthio)benzylthio)-[1,2,4]triazolo[4,3-a]pyrazin-8(7H)-one;
53 7-(3-chloro-2-methylphenyl)-3-(2-nitrobenzylthio)-[1,2,4]triazolo[4,3-a]pyrazin-8(7H)-one;
54 3-(biphenyl-2-ylmethylthio)-7-(3-chloro-2-methylphenyl)-[1,2,4]triazolo[4,3-a]pyrazin-8(7H)-one;
55 7-(3-chloro-2-methylphenyl)-3-(2-fluoro-3-methylbenzylthio)-[1,2,4]triazolo[4,3-a]pyrazin-8(7H)-one;
56 3-(2-fluorobenzylthio)-7-(2-methyl-3-(trifluoromethyl)phenyl)-[1,2,4]triazolo[4,3-a]pyrazin-8(7H)-one;
57 7-(2-methyl-3-(trifluoromethyl)phenyl)-3-(2-methylbenzylthio)-[1,2,4]triazolo[4,3-a]pyrazin-8(7H)-one;
58 3-(3,5-dichlorobenzylthio)-7-(2-fluoro-3-(trifluoromethyl)phenyl)-[1,2,4]triazolo[4,3-a]pyrazin-8(7H)-one;
59 3-(benzylthio)-7-(2-fluoro-3-(trifluoromethyl)phenyl)-[1,2,4]triazolo[4,3-a]pyrazin-8(7H)-one;
60 7-(2-chloro-3-(trifluoromethyl)phenyl)-3-(3,5-dichlorobenzylthio)-[1,2,4]triazolo[4,3-a]pyrazin-8(7H)-one;
61 3-(benzylthio)-7-(2-chloro-3-(trifluoromethyl)phenyl)-[1,2,4]triazolo[4,3-a]pyrazin-8(7H)-one;
62 7-(2-methyl-3-(trifluoromethyl)phenyl)-3-(1-phenylethylthio)-[1,2,4]triazolo[4,3-a]pyrazin-8(7H)-one;
63 3-(3-chloro-2-fluorobenzylthio)-7-(3-chloro-2-methylphenyl)-[1,2,4]triazolo[4,3-a]pyrazin-8(7H)-one;
64 3-(5-chloro-2-fluorobenzylthio)-7-(3-chloro-2-methylphenyl)-[1,2,4]triazolo[4,3-a]pyrazin-8(7H)-one;
65 3-(benzylthio)-7-(2-chloro-3-fluorophenyl)-[1,2,4]triazolo[4,3-a]pyrazin-8(7H)-one;
66 7-(3-chloro-2-methylphenyl)-3-(2,3-dichlorobenzylthio)-[1,2,4]triazolo[4,3-a]pyrazin-8(7H)-one;
67 3-(3,5-dichlorobenzylthio)-7-(2,3-dichlorophenyl)-[1,2,4]triazolo[4,3-a]pyrazin-8(7H)-one;
68 7-(3-chloro-2-methylphenyl)-3-(2-fluoro-6-(trifluoromethyl)benzylthio)-[1,2,4]triazolo[4,3-a]pyrazin-8(7H)-one;
69 3-(3,5-dichlorobenzylthio)-7-(2-methyl-3-(trifluoromethyl)phenyl)-[1,2,4]triazolo[4,3-a]pyrazin-8(7H)-one;
70 7-(3-chloro-2-methylphenyl)-3-(2-(trifluoromethoxy)benzylthio)-[1,2,4]triazolo[4,3-a]pyrazin-8(7H)-one;
71 3-(2-bromobenzylthio)-7-(3-chloro-2-methylphenyl)-[1,2,4]triazolo[4,3-a]pyrazin-8(7H)-one;
72 7-(3-chloro-2-methylphenyl)-3-(2,5-dichlorobenzylthio)-[1,2,4]triazolo[4,3-a]pyrazin-8(7H)-one;
73 7-(3-chloro-2-methylphenyl)-3-(3-methylbenzylthio)-[1,2,4]triazolo[4,3-a]pyrazin-8(7H)-one;
74 7-(3-chloro-2-methylphenyl)-3-(2-iodobenzylthio)-[1,2,4]triazolo[4,3-a]pyrazin-8(7H)-one;
75 7-(3-chloro-2-methylphenyl)-3-(2,6-dichlorobenzylthio)-[1,2,4]triazolo[4,3-a]pyrazin-8(7H)-one;
76 7-(3-chloro-2-methylphenyl)-3-(2,6-dimethylbenzylthio)-[1,2,4]triazolo[4,3-a]pyrazin-8(7H)-one;
77 7-(3-chloro-2-methylphenyl)-3-(3-iodobenzylthio)-[1,2,4]triazolo[4,3-a]pyrazin-8(7H)-one;
78 7-(3-chloro-2-methylphenyl)-3-(3-chloro-5-(trifluoromethyl)benzylthio)-[1,2,4]triazolo[4,3-a]pyrazin-8(7H)-one;
79 7-(3-chloro-2-methylphenyl)-3-(3,5-dimethoxybenzylthio)-[1,2,4]triazolo[4,3-a]pyrazin-8(7H)-one;
80 3-(benzylthio)-7-(3-chloro-2-methoxyphenyl)-[1,2,4]triazolo[4,3-a]pyrazin-8(7H)-one;
81 3-(benzylthio)-7-(2-chlorophenyl)-[1,2,4]triazolo[4,3-a]pyrazin-8(7H)-one;
82 3-(benzylthio)-7-(3-chloro-2-fluorophenyl)-[1,2,4]triazolo[4,3-a]pyrazin-8(7H)-one;
83 7-(3-bromo-2-methylphenyl)-3-(3,5-dichlorobenzylthio)-[1,2,4]triazolo[4,3-a]pyrazin-8(7H)-one;
84 7-(3-chloro-2-methylphenyl)-3-(3,5-dibromobenzylthio)-[1,2,4]triazolo[4,3-a]pyrazin-8(7H)-one;
85 7-(3-chloro-2-methylphenyl)-3-(3-(trifluoromethoxy)benzylthio)-[1,2,4]triazolo[4,3-a]pyrazin-8(7H)-one;
86 3-(3-bromobenzylthio)-7-(3-chloro-2-methylphenyl)-[1,2,4]triazolo[4,3-a]pyrazin-8(7H)-one;
87 2-((7-(3-chloro-2-methylphenyl)-8-oxo-7,8-dihydro-[1,2,4]triazolo[4,3-a]pyrazin-3-ylthio)methyl)benzonitrile;
88 3-(3,5-bis(trifluoromethyl)benzylthio)-7-(3-chloro-2-methylphenyl)-[1,2,4]triazolo[4,3-a]pyrazin-8(7H)-one;
89 7-(3-chloro-2-methylphenyl)-3-(3-(trifluoromethyl)benzylthio)-[1,2,4]triazolo[4,3-a]pyrazin-8(7H)-one;
90 methyl 2-((7-(3-chloro-2-methylphenyl)-8-oxo-7,8-dihydro-[1,2,4]triazolo[4,3-a]pyrazin-3-ylthio)methyl)benzoate;
91 methyl 3-((7-(3-chloro-2-methylphenyl)-8-oxo-7,8-dihydro-[1,2,4]triazolo[4,3-a]pyrazin-3-ylthio)methyl)benzoate;
92 3-(benzylthio)-7-(2,3-dimethylphenyl)-[1,2,4]triazolo[4,3-a]pyrazin-8(7H)-one;
93 3-(benzylthio)-7-(2-methyl-3-(trifluoromethyl)phenyl)-[1,2,4]triazolo[4,3-a]pyrazin-8(7H)-one;
94 7-(3-chloro-2-methylphenyl)-3-((2-methylthiazol-4-yl)methylthio)-[1,2,4]triazolo[4,3-a]pyrazin-8(7H)-one;
95 3-(benzylthio)-7-(2,3-dichlorophenyl)-[1,2,4]triazolo[4,3-a]pyrazin-8(7H)-one;
96 7-(3-chloro-2-methylphenyl)-3-(pyridin-3-ylmethylthio)-[1,2,4]triazolo[4,3-a]pyrazin-8(7H)-one;
97 7-(3-chloro-2-methylphenyl)-3-(naphthalen-2-ylmethylthio)-[1,2,4]triazolo[4,3-a]pyrazin-8(7H)-one;
98 7-(3-chloro-2-methylphenyl)-3-(pyridin-2-ylmethylthio)-[1,2,4]triazolo[4,3-a]pyrazin-8(7H)-one;
99 3-(benzylthio)-7-(3-bromo-2-methylphenyl)-[1,2,4]triazolo[4,3-a]pyrazin-8(7H)-one;
100 3-(benzylthio)-7-(3,4-dichlorophenyl)-[1,2,4]triazolo[4,3-a]pyrazin-8(7H)-one;
101 7-(3-chloro-2-methylphenyl)-3-(3,5-dichlorobenzylthio)-[1,2,4]triazolo[4,3-a]pyrazin-8(7H)-one;
102 7-(3-chloro-2-methylphenyl)-3-(3,4,5-trifluorobenzylthio)-[1,2,4]triazolo[4,3-a]pyrazin-8(7H)-one;
103 7-(3-chloro-2-methylphenyl)-3-(3,4-dichlorobenzylthio)-[1,2,4]triazolo[4,3-a]pyrazin-8(7H)-one;
104 7-(3-chloro-2-methylphenyl)-3-(4-isopropylbenzylthio)-[1,2,4]triazolo[4,3-a]pyrazin-8(7H)-one;
105 7-(3-chloro-2-methylphenyl)-3-(2,6-difluorobenzylthio)-[1,2,4]triazolo[4,3-a]pyrazin-8(7H)-one;
106 7-(3-chloro-2-methylphenyl)-3-(perfluorobenzylthio)-[1,2,4]triazolo[4,3-a]pyrazin-8(7H)-one;
107 tert-butyl 2-(7-(3-chloro-2-methylphenyl)-8-oxo-7,8-dihydro-[1,2,4]triazolo[4,3-a]pyrazin-3-ylthio)acetate;
108 7-(3-chloro-2-methylphenyl)-3-(2-(2-methoxyphenyl)-2-oxoethylthio)-[1,2,4]triazolo[4,3-a]pyrazin-8(7H)-one;
109 7-(3-chloro-2-methylphenyl)-3-(2-oxo-2-(2-(trifluoromethyl)phenyl)ethylthio)-[1,2,4]triazolo[4,3-a]pyrazin-8(7H)-one;
110 7-(3-chloro-2-methylphenyl)-3-(2-methoxybenzylthio)-[1,2,4]triazolo[4,3-a]pyrazin-8(7H)-one;
111 7-(3-chloro-2-methylphenyl)-3-(2-(trifluoromethyl)benzylthio)-[1,2,4]triazolo[4,3-a]pyrazin-8(7H)-one;
112 7-(3-chloro-2-methylphenyl)-3-(phenethylthio)-[1,2,4]triazolo[4,3-a]pyrazin-8(7H)-one;
113 7-(3-chloro-2-methylphenyl)-3-(4-methoxybenzylthio)-[1,2,4]triazolo[4,3-a]pyrazin-8(7H)-one;
114 7-(3-chloro-2-methylphenyl)-3-(3,5-difluorobenzylthio)-[1,2,4]triazolo[4,3-a]pyrazin-8(7H)-one;
115 7-(3-chloro-2-methylphenyl)-3-(3,4-difluorobenzylthio)-[1,2,4]triazolo[4,3-a]pyrazin-8(7H)-one;
166 3-(biphenyl-2-ylmethylamino)-7-(3-chloro-2-methylphenyl)-[1,2,4]triazolo[4,3-a]pyrazin-8(7H)-one;
167 3-(benzhydrylamino)-7-(3-chloro-2-methylphenyl)-[1,2,4]triazolo[4,3-a]pyrazin-8(7H)-one;
168 7-(3-chloro-2-methylphenyl)-3-(phenethylamino)-[1,2,4]triazolo[4,3-a]pyrazin-8(7H)-one;
169 3-(benzyl(methyl)amino)-7-(3-chloro-2-methylphenyl)-[1,2,4]triazolo[4,3-a]pyrazin-8(7H)-one;
170 7-(3-chloro-2-methylphenyl)-3-(dibenzylamino)-[1,2,4]triazolo[4,3-a]pyrazin-8(7H)-one; and
171 3-(benzyl(cyclopropylmethyl)amino)-7-(3-chloro-2-methylphenyl)-[1,2,4]triazolo[4,3-a]pyrazin-8(7H)-one.

13. A pharmaceutical composition comprising a therapeutically effective amount of the compound of claim 1 or a pharmaceutically acceptable salt thereof and a pharmaceutically acceptable carrier.

14. A method of treating pain or an inflammatory disease, comprising administering a therapeutically effective amount of the compound of claim 1 or a pharmaceutically acceptable salt thereof to a patient in need thereof.

15. A method of treating pain or an inflammatory disease, comprising administering the compound of claim 1 or a pharmaceutically acceptable salt thereof and an additional therapeutic agent useful in treating pain or an inflammatory disease to a patient in need thereof.

16. A pharmaceutical composition comprising a therapeutically effective amount of the compound of claim 12 or a pharmaceutically acceptable salt thereof and a pharmaceutically acceptable carrier.

17. A method of treating pain or an inflammatory disease, comprising administering a therapeutically effective amount of the compound of claim 12 or a pharmaceutically acceptable salt thereof to a patient in need thereof.

18. A method of treating pain or an inflammatory disease, comprising administering the compound of claim 12 or a pharmaceutically acceptable salt thereof and an additional therapeutic agent useful in treating pain or an inflammatory disease to a patient in need thereof.
